(19) United States
(12) Patent Application Publication
(10) Pub. No.: US 2012/0276606 A1
(43) Pub. Date: Nov. 1, 2012

(54) RECOMBINANT MICROORGANISMS WITH 1,3-BUTANEDIOL-PRODUCING FUNCTION AND USES THEREOF

(75) Inventors: Tomohito Okabayashi, Hyogo (JP); Takanori Nakajima, Niigata (JP); Hiroaki Yamamoto, Niigata (JP)

(73) Assignee: Daicel Corporation, Osaka (JP)

(21) Appl. No.: 13/504,391

(22) PCT Filed: Oct. 29, 2010
(86) PCT No.: PCT/JP2010/069274

§ 371 Date: Jul. 19, 2012

(30) Foreign Application Priority Data
Oct. 30, 2009 (JP) 2009-251276

(51) Int. Cl. C12N 1/21; C12N 1/15; C12P 7/18; C12N 1/19

(52) U.S. Cl. 435/158; 435/252.33; 435/252.31; 435/252.34; 435/252.3; 435/252.32; 435/252.35; 435/254.2; 435/254.21; 435/254.23; 435/254.22; 435/254.4; 435/254.3; 435/254.6; 435/254.11

(57) ABSTRACT

An objective of the present invention is to provide recombinant microorganisms efficiently producing optically active 1,3-butanediol, which is useful as a material for synthesizing pharmaceuticals and such, and provide methods for efficiently producing optically active 1,3-butanediol using the recombinant microorganisms. As a result of dedicated research for achieving the above objective, the present inventors succeeded in producing recombinant microorganisms in which the activity of an enzyme catalyzing the reduction reaction represented by Formula 1 is enhanced and which produce a diol compound represented by Formula 2.

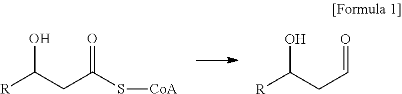

(wherein R represents a C₁₋₃ alkyl group or hydrogen)

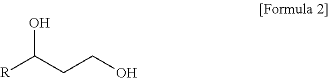

(wherein R represents a C₁₋₃ alkyl group or hydrogen)

### TECHNICAL FIELD

The present invention is related to recombinant microorganisms with a 1,3-butanediol-producing function, and methods for producing 1,3-butanediol using the microorganisms.

### BACKGROUND ART

1,3-Butanediol is useful as a chemical with various applications, such as moisturizers, resin materials, surfactants, moisture absorbents, and solvents, and as a raw material thereof. Also, its optically active molecules, (R)-1,3-butanediol and (S)-1,3-butanediol, are useful as raw materials for synthesizing pharmaceuticals, agrochemicals, and such.

Conventionally, 1,3-butanediol is made by chemical production in which acetaldehyde chemically manufactured from petroleum, a fossil resource, is used as a raw material to produce acetaldol, which is then hydrogenated. Meanwhile, optically active 1,3-butanediol can be produced by methods represented by Patent Document 1, in which (R)- or (S)-1,3-butanediol is produced by allowing a microorganism capable of preferentially assimilating either its (S)- or (R)-isomer, such as Candida palapsilosis or Kluyveromyces lacitis, to act on racemic 1,3-butanediol chemically synthesized from fossil resources, and then collecting the remaining enantiomer.

In other methods represented by Patent Document 2, (R)- or (S)-1,3-butanediol is produced by allowing a microorganism such as Kluyveromyces lactis or Candida palapsilosis to act on 4-hydroxy-2-butanone chemically synthesized from fossil resources, and utilizing the asymmetric reduction activity of the microorganism.

Furthermore, there are methods of producing optically active 1,3-butanediol in one or two steps using stereo-selective oxidoreductases or recombinant bacteria overexpressing such enzymes. Such methods include: the production of (R)-1,3-butanediol from the racemic form using a (S)-specific secondary alcohol dehydrogenase derived from Geotricum sp. (Non-patent Document 1); the production of (R)-1,3-butanediol from the racemic form using recombinant Escherichia coli expressing a (S)-specific secondary alcohol dehydrogenase derived from Candida palapsilosis (Patent Document 3), the production of (R)-1,3-butanediol from 4-hydroxy-2-butanone or the production of (S)-1,3-butanediol from the racemic form using recombinant E. coli expressing an (R)-specific 2,3-butanediol dehydrogenase derived from Kluyveromyces lactis (Patent Document 4). However, all these methods use a non-natural compound as a substrate, which needs to be chemically synthesized.

Meanwhile, in Non-patent Document 2, 1,3-butanediol was detected in the culture fluid of Geotricum fragrans cultured in a medium containing cassava waste. However, 1,3-butanediol was found as one of the volatile substances, and the culture was not intended to produce 1,3-butanediol.

In recent years, from the view point of the depletion of fossil resources and global warming, there has been an increasing social demand for establishment of chemical production systems that use biomass (botanical resource)—derived materials as renewable resources.

For example, it is known that solvents can be produced from glucose, one of the biomass-derived materials, via a CoA-derivative using the acetone-butanol fermentation pathway, as represented by Clostridium acetobutylicum. For the type strain of this species, C. acetobutylicum ATCC824, the entire genomic DNA sequence has been sequenced, and a solvent-producing gene characteristic of acetone-butanol fermentation bacteria, adhE (an aldehyde-alcohol dehydrogenase that has the functions of EC1.2.1.10 and EC1.1.1.1), has been revealed (Non-patent Document 3). There has been few enzymological reports for the gene product of adhE derived from C. acetobutylicum (aldehyde-alcohol dehydrogenase that has the functions of EC1.2.1.10 and EC1.1.1.1), and only the assessment of the cell-free extract of C. acetobutylicum DSM1732 has been performed for the butanol dehydrogenase activity using butanol or butylaldehyde as a substrate and for the butylaldehyde dehydrogenase activity using butylaldehyde or butyryl-CoA as a substrate, in Non-patent Document 4. The adhE gene product has never been used for other purposes than producing 1-butanol as represented by Non-patent Document 5.

There has been no report of producing 1,3-butanediol from biomass-derived materials, and there has been a demand for a method of producing 1,3-butanediol from renewable resources such as biomass.

### PRIOR ART DOCUMENTS

### Patent Documents

[Patent Document 1] Japanese Patent Application Kokai Publication No. (JP-A) H02-195897 (unexamined, published Japanese patent application)
[Patent Document 2] JP-A (Kokai) H02-31684
[Patent Document 3] JP-A (Kokai) 2000-197485
[Patent Document 4] JP-A (Kokai) 2002-345479

### Non-Patent Documents

[Non-patent Document 1] Biosci. Biotech. Biochem., 1996, 60(7), 1191-1192
[Non-patent Document 2] Proc. Biochem., 2003, 39, 411-414
[Non-patent Document 3] J. Bacteriol., 2001, 183(16), 4823-4838
[Non-patent Document 4] Appl. Microbial. Biotechnol., 1987, 26, 268-272
[Non-patent Document 5] Metabol. Engineer., 2008, 10, 305-311

### SUMMARY OF THE INVENTION

### Problems to be Solved by the Invention

An objective of the present invention is to provide microorganisms capable of producing 1,3-butanediol from renewable resources of biomass (botanical resources). Another objective of the present invention is to provide methods by which 1,3-butanediol can be produced using microorganisms having a desired function(s).

### Means for Solving the Problems

As a result of dedicated research to develop a method for producing 1,3-butanediol from biomass, which is a renewable resource, the present inventors have discovered that AdhE derived from Clostridium acetobutylicum (CaAdhE) not only generates acetaldehyde and butylaldehyde by reducing acetyl-CoA and butyryl-CoA in an NADH-dependent manner, but also surprisingly generates 3-hydroxybutylaldehyde by reducing 3-hydroxybutyryl-CoA, which has a hydroxyl group at position 3. The present inventors have also discovered that CaAdhE has an activity of reducing 3-hydroxybutylaldehyde in an NADH-dependent manner, thereby catalyzing a reaction of producing 1,3-butanediol. They have successfully achieved efficient production of 1,3-butanediol by culturing, with glucose as a carbon source, recombinant E. coli capable of coexpressing CaAdhE with β-ketothiolase, which catalyzes the reaction of generating acetoacetyl-CoA from two molecules of acetyl-CoA, and 3-hydroxybutyryl-CoA dehydrogenase, which catalyzes the reaction of reducing the 3-carbonyl group of acetoacetyl-CoA in an NAD(P)H-dependent manner and thereby generating 3-hydroxybutyryl-CoA.

Based on the above findings, the present inventors have succeeded in producing 1,3-butanediol by allowing the reaction of Formula 8 shown below to occur in microorganisms carrying the above enzymes, thereby completing the present invention.

Thus, the present invention provides 1,3-butanediol-producing microorganisms. Also, the present invention provides methods for efficiently producing 1,3-butanediol using such microorganisms.

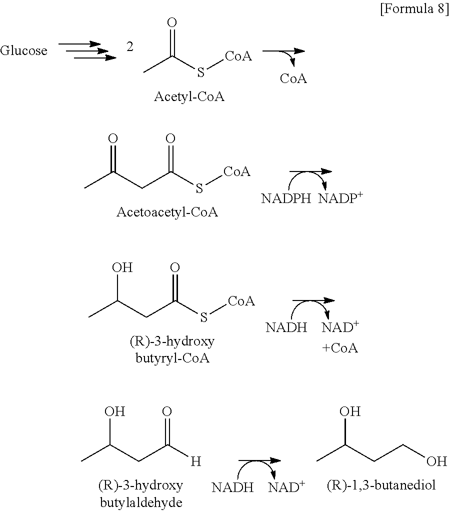

More specifically, the present inventions provides:

[1] a recombinant microorganism in which the enzymatic activity of (1) shown below is enhanced, wherein the microorganism produces 1,3-alkyldiol represented by Formula 2 from a fermentation substrate:
(1) activity of an enzyme that catalyzes production of 3-hydroxyalkylaldehyde by reducing 3-hydroxyacyl-CoA using NADH and/or NADPH as a coenzyme, as shown by Formula 1:

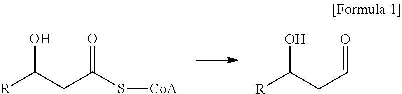

(wherein R represents a C₁₋₃ alkyl group or hydrogen; and CoA represents coenzyme A)

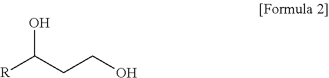

(wherein R represents a C₁₋₃ alkyl group or hydrogen);
[2] the recombinant microorganism of [1], wherein R is methyl in Formulas 1 and 2 recited in [1], and the microorganism produces 1,3-butanediol from a fermentation substrate;
[3] the recombinant microorganism of [1] or [2], wherein 1,3-butanediol produced from the fermentation substrate recited in [2] is (R)- or (S)-1,3-butanediol;
[4] the recombinant microorganism of any one of [1] to [3], wherein the enzyme catalyzing the reaction of Formula 1 recited in [1] (1) is classified as EC1.2.1.10 under the international classification of enzyme;
[5] the recombinant microorganism of any one of [1] to [4], wherein the enzyme catalyzing the reaction of Formula 1 recited in [1] (1) is any one of:
(a) a protein comprising the amino acid sequence of SEQ ID NO: 1, 65, or 67;
(b) an enzyme comprising an amino acid sequence in which one or more amino acids are substituted, deleted, inserted, or added in the amino acid sequence of SEQ ID NO: 1, 65, or 67;
(c) an enzyme comprising an amino acid sequence encoded by the nucleotide sequence of SEQ ID NO: 2, 66, or 68;
(d) an enzyme comprising an amino acid sequence encoded by a DNA Which hybridizes under stringent conditions with a DNA comprising the nucleotide sequence of SEQ ID NO: 2, 66, or 68; and
(e) a protein having an identity of 85% or higher with the amino acid sequence of SEQ ID NO: 1, 65, or 67;
[6] the recombinant microorganism of any one of [1] to [5], in which the enzymatic activity of (2) shown below is enhanced in addition to the enzymatic activity of (1) recited in [1], wherein the microorganism produces 1,3-alkyldiol represented by Formula 2 from a fermentation substrate:
(1) activity of an enzyme that catalyzes production of 3-hydroxyalkylaldehyde by reducing 3-hydroxyacyl-CoA using NADH and/or NADPH as a coenzyme, as shown by Formula 1; and
(2) activity of an enzyme that catalyzes production of 1,3-alkyldiol represented by Formula 2 by reducing 3-hydroxyalkylaldehyde using NADH and/or NADPH as a coenzyme, as shown by Formula 3 (Formula 3 does not show two reactions, but only shows the production of alcohol from aldehyde):

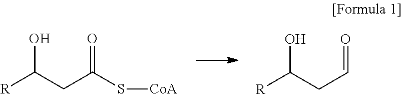

(wherein R represents a C₁₋₃ alkyl group or hydrogen; and CoA represents coenzyme A)

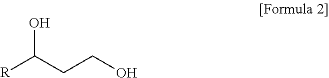

(wherein R represents a C₁₋₃ alkyl group or hydrogen)

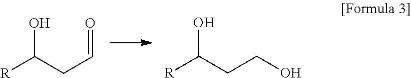

(wherein R represents a C₁₋₃ alkyl group or hydrogen);
[7] the recombinant microorganism of [6], wherein R is methyl in Formulas 1 to 3 recited in [6], and the microorganism produces 1,3-butanediol from a fermentation substrate;
[8] the recombinant microorganism of [6] or [7], wherein 1,3-butanediol produced from the fermentation substrate recited in [7] is (R)- or (S)-1,3-butanediol;
[9] the recombinant microorganism of any one of [6] to [8], wherein the enzyme catalyzing the reaction of Formula 3 recited in [6] is any one of:
(a) a protein comprising the amino acid sequence of SEQ ID NO: 3, 5, or 7;
(b) an enzyme comprising an amino acid sequence in which one or more amino acids are substituted, deleted, inserted, or added in the amino acid sequence of SEQ ID NO: 3, 5, or 7;
(c) an enzyme comprising an amino acid sequence encoded by the nucleotide sequence of SEQ ID NO: 4, 6, or 8;
(d) an enzyme comprising an amino acid sequence encoded by a DNA which hybridizes under stringent conditions with a DNA comprising the nucleotide sequence of SEQ ID NO: 4, 6, or 8; and
(e) a protein having an identity of 85% or higher with the amino acid sequence of SEQ ID NO: 3, 5, or 7;
[10] the recombinant microorganism of any one of [1] to [5], in which the enzymatic activity of (3) shown below is enhanced in addition to the enzymatic activity of (1) recited in [1], wherein the microorganism produces 1,3-alkyldiol represented by Formula 2 from a fermentation substrate:
(1) activity of an enzyme that catalyzes production of 3-hydroxyalkylaldehyde by reducing 3-hydroxyacyl-CoA using NADH and/or NADPH as a coenzyme, as shown by Formula 1; and
(3) activity of an enzyme that produces 3-hydroxyacyl-CoA by reducing 3-oxoacyl-CoA in an NADH- and/or NADPH-dependent manner as shown by Formula 4:

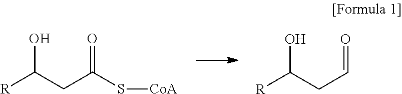

(wherein R represents a C₁₋₃ alkyl group or hydrogen; and CoA represents coenzyme A)

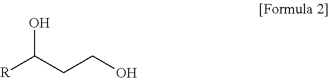

(wherein R represents a C₁₋₃ alkyl group or hydrogen)

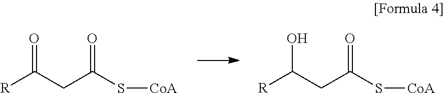

(wherein R represents a C₁₋₃ alkyl group or hydrogen; and CoA represents coenzyme A);
[11] the recombinant microorganism of [10], wherein R is methyl in Formulas 1 and 2 recited in
[10], and the microorganism produces (R)-1,3-butanediol represented by Formula 5 from a fermentation substrate:

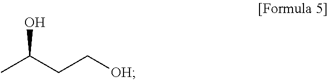

[12] the recombinant microorganism of [10] or [11], wherein the enzyme catalyzing the reaction of Formula 4 recited in [10] is an R-form-specific reductase, and is any one of:
(a) a protein comprising the amino acid sequence of SEQ ID NO: 9, 11, 13, 15, or 17;
(b) an enzyme comprising an amino acid sequence in which one or more amino acids are substituted, deleted, inserted, or added in the amino acid sequence of SEQ ID NO: 9, 11, 13, 15, or 17;
(c) an enzyme comprising an amino acid sequence encoded by the nucleotide sequence of SEQ ID NO: 10, 12, 14, 16, or 18;
(d) an enzyme comprising an amino acid sequence encoded by a DNA which hybridizes under stringent conditions with a DNA comprising the nucleotide sequence of SEQ ID NO: 10, 12, 14, 16, or 18; and
(e) an enzyme having an identity of 85% or higher with the amino acid sequence of SEQ ID NO: 9, 11, 13, 15, or 17;
[13] the recombinant microorganism of [10], wherein R is methyl in Formulas 1 and 2 recited in [10], and the microorganism produces (S)-1,3-butanediol represented by Formula 6 from a fermentation substrate:

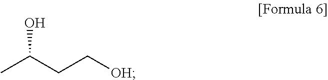

[14] the recombinant microorganism of [10] or [13], wherein the enzyme catalyzing the reaction of Formula 4 recited in [10] is an S-form-specific reductase, and is any one of
(a) a protein comprising the amino acid sequence of SEQ ID NO: 19;
(b) an enzyme comprising an amino acid sequence in which one or more amino acids are substituted, deleted, inserted, or added in the amino acid sequence of SEQ ID NO: 19;
(c) an enzyme comprising an amino acid sequence encoded by the nucleotide sequence of SEQ ID NO: 20:
(d) an enzyme comprising an amino acid sequence encoded by a DNA which hybridizes under stringent conditions with a DNA comprising the nucleotide sequence of SEQ ID NO: 20; and
(e) an enzyme having an identity of 85% or higher with the amino acid sequence of SEQ ID NO: 19;
[15] the recombinant microorganism of any one of [1] to [5], in which the enzymatic activity of (4) shown below is enhanced in addition to the enzymatic activity of (1) recited in [1], wherein the microorganism produces (R)- or (S)-1,3-butanediol represented by Formula 2 from a fermentation substrate:
(1) activity of an enzyme that catalyzes production of 3-hydroxybutylaldehyde by reducing 3-hydroxybutyryl-CoA using NADH and/or NADPH as a coenzyme, as shown by Formula 1; and
(4) activity of a β-ketothiolase that catalyzes production of acetoacetyl-CoA from two molecules of acetyl-CoA as shown by Formula 7:

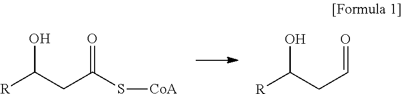

(wherein R represents methyl; and CoA represents coenzyme A)

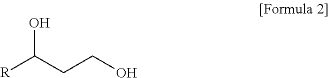

(wherein R represents methyl)

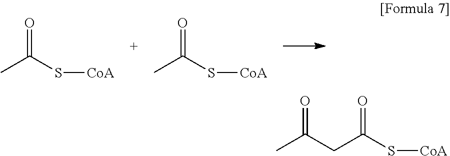

(wherein CoA represents coenzyme A);

[16] the recombinant microorganism of [15], wherein the enzyme catalyzing the reaction of Formula 7 recited in [15] is any one of:
(a) a protein comprising the amino acid sequence of SEQ ID NO: 21, 23, or 25;
(b) an enzyme comprising an amino acid sequence in which one or more amino acids are substituted, deleted, inserted, or added in the amino acid sequence of SEQ ID NO: 21, 23, or 25;
(c) an enzyme comprising an amino acid sequence encoded by the nucleotide sequence of SEQ ID NO: 22, 24, or 26;
(d) an enzyme comprising an amino acid sequence encoded by a DNA which hybridizes under stringent conditions with a DNA comprising the nucleotide sequence of SEQ ID NO: 22, 24, or 26; and
(e) an enzyme having an identity of 85% or higher with the amino acid sequence of SEQ ID NO: 21, 23, or 25;
[17] the recombinant microorganism of any one of [1] to [5], in which the enzymatic activities of (2) to (4) shown below are enhanced in addition to the enzymatic activity of (1) recited in [1], wherein the microorganism produces (R)- or (S)-1,3-butanediol represented by Formula 2 from a fermentation substrate:
(1) activity of an enzyme that catalyzes production of 3-hydroxybutylaldehyde by reducing β-hydroxybutyryl-CoA using NADH and/or NADPH as a coenzyme, as shown by Formula 1;
(2) activity of an enzyme that catalyzes production of 1,3-butanediol represented by Formula 2 by reducing 3-hydroxybutylaldehyde using NADH and/or NADPH as a coenzyme, as shown by Formula 3 (Formula 3 does not show two reactions, but only shows the production of alcohol from aldehyde);
(3) activity of an enzyme that produces 3-hydroxybutyryl-CoA by reducing acetoacetyl-CoA in an NADH- and/or NADPH-dependent manner as shown by Formula 4;
(4) activity of a β-ketothiolase that catalyzes production of acetoacetyl-CoA from two molecules of acetyl-CoA as shown by Formula 7:

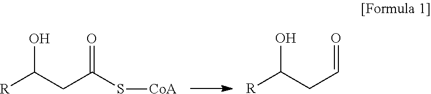

(wherein R represents methyl; and CoA presents coenzyme A)

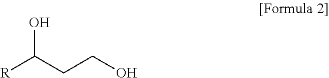

(wherein R represents methyl)

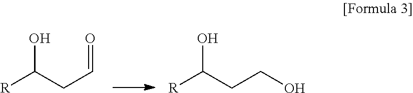

(wherein R represents methyl)

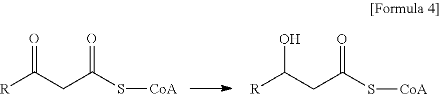

(wherein R represents methyl, and CoA represents coenzyme A)

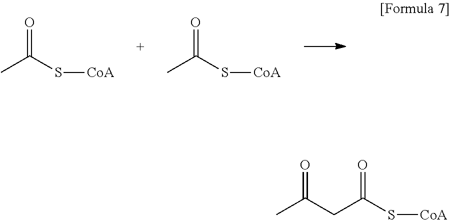

(wherein CoA represents coenzyme A);
[18] the recombinant microorganism of any one of [1] to [17], wherein the host cell is Escherichia coli;
[19] a method for producing a diol compound represented by Formula 2, comprising the steps of

contacting a fermentation substrate with at least one active material selected from the group consisting of a culture of the recombinant microorganism of any one of [1] to [18], a cell of the recombinant microorganism, and a processed product thereof; and

collecting 1,3-alkyodiol represented by Formula 2:

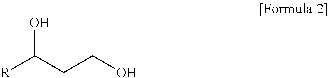

(wherein R represents a C₁₋₃ alkyl group or hydrogen);
[20] the method of [19], wherein the diol compound is (R)-1,3-butanediol represented by Formula 5.

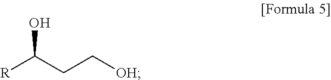

[21] the method of [19], wherein the diol compound is (S)-1,3-butanediol represented by Formula 6;
[22] the method of any one of [19] to [21], wherein the fermentation substrate is selected from the group consisting of sugars and glycerol;
[23] the method of [22], wherein the fermentation substrate is selected from the group consisting of glucose, lactose, xylose, sucrose, and glycerol; and
[24] the method of any one of [19] to [23], wherein the culturing of the recombinant microorganism and the production of the diol compound are carried out separately.

### BRIEF DESCRIPTION OF THE DRAWINGS

FIG. 1 shows the production of a plasmid containing the entire ReTHL gene.

FIG. 2 shows the production of a plasmid containing the entire ZrTHL gene.

FIG. 3 shows the production of a plasmid containing the entire EcTHL gene.

FIG. 4 shows the production of a plasmid containing the entire ReTHL and ReAR1 genes.

FIG. 5 shows the production of a plasmid containing the entire ReTHL and ZrAR1 genes.

FIG. 6 shows the production of a plasmid containing the entire ReTHL and SvKR1 genes.

FIG. 7 shows the production of a plasmid containing the entire ReTHL and BstKR1 genes.

FIG. 8 shows the production of a plasmid containing the entire ReTHL and CaHBD genes.

FIG. 9 shows the production of a plasmid containing the entire ReTHL and PfODH genes.

FIG. 10 shows the production of a plasmid containing the entire CaAdhE gene.

FIG. 11 shows the production of a plasmid containing the entire TpAdhE gene.

FIG. 12 shows the production of a plasmid containing the entire PfALD gene.

FIG. 13 shows the production of a plasmid containing the entire ReTHL, ReAR1, and CaAdhE genes.

FIG. 14 shows the production of a plasmid containing the entire CaBDHB gene.

FIG. 15 shows the production of a plasmid containing the entire ReTHL, ReAR1, CaAdhE, and CaBDHB genes.

### MODE FOR CARRYING OUT THE INVENTION

The present invention is related to recombinant microorganisms in which the enzymatic activity of (1) shown below is enhanced, and which produce a diol compound represented by Formula 2 (1,3-alkyldiol) from a fermentation substrate.

(1) activity of an enzyme that catalyzes production of 3-hydroxyalkylaldehyde by reducing 3-hydroxyacyl-CoA using NADH and/or NADPH as a coenzyme, as shown by Formula 1:

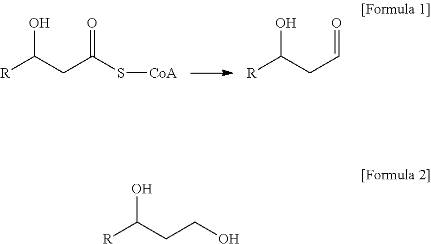

In the present invention, the diol compound represented by Formula 2 is preferably produced via the reduction reaction of Formula 1. In the present invention, R in Formula 1 is preferably a C₁₋₃ alkyl group (methyl group, ethyl group, propyl group, or isopropyl group) or hydrogen. In Formula 1, CoA represents coenzyme A.

Preferred diol compounds produced in the present invention include compounds of Formula 2 in which, for example, R is a C₁₋₃ alkyl group (methyl group, ethyl group, propyl group, or isopropyl group) or hydrogen. More specifically, preferred examples of the diol compounds produced in the present invention include 1,3-butanediol (where R is methyl in both Formulas 1 and 2). The optical activity of 1,3-butanediol produced in the present invention is not particularly limited, and the R-form and S-form of 1,3-butanediol ((R)- and (S)-1,3-butanediol) are included in 1,3-butanediol produced in the present invention.

In the present invention, the enzymes that produce 3-hydroxybutylaldehyde by reducing 3-hydroxybutyryl-CoA using and/or NADPH as a coenzyme as shown by Formula 1 include enzymes classified as EC1.2.1.10, which have the systematic name “acetaldehyde:NAD⁺ oxidoreductase (CoA-acetylating)” given by the International Union of Biochemistry and Molecular Biology (IUBMB) (http://www.chem.qmul.ac.uk/iubmb/), and catalyze the following reaction: aldehyde+CoA+NAD⁺=acyl-CoA+NADH+H⁺. EC numbers are sets of four numbers for systematically classifying enzymes according to their reaction schemes, and are defined by the Enzyme Commission of IUBMB. Specifically, the above enzymes include those of microorganisms having a butanol fermentation pathway which catalyze the production of butylaldehyde by NADH- or NADPH-dependent reduction of butyryl-CoA (for example, butylaldehyde dehydrogenase) in the butanol biosynthesis pathway. Genes encoding these enzymes or enzymes catalyzing a similar reaction are generally termed adhE.

More specifically, the above enzymes include gene products of adhE derived from Clostridium bacteria such as Clostridium acetobutylicum, Clostridium beijerinekii, Clostridium saccharoacetobutylicum, and Clostridium saccharoperbutylacetonicum.

Preferred enzymes having the function of EC1.2.1.10 are enzymes with the recommended name “aldehyde-alcohol dehydrogenase” according to UniProt (http://www.uniprot.org/), including bifunctional enzymes catalyzing both the reaction of producing aldehyde from acyl-CoA and the reaction of producing alcohol from aldehyde, and enzymes only catalyzing the reaction of producing aldehyde from acyl-CoA. Specifically, the gene product of adhE1 for the gene product of adhE derived from Clostridium acetobutylicum can be used for the present purposes. The adhE gene and mhpF gene possessed by Escherichia coli, lactic acid bacteria, and such can also be used. Specifically, the adhE gene and mhpF gene derived from Escherichia coli, and the adhE gene product derived from Leuconostoc mesenteroides can be used. In addition to the above-described genes, genes suitable thr the present purposes can also be selected from microorganisms whose genomic DNA has been sequenced. Specifically, genes from Thermoanaerobacter pseudethanolicus and Propionibacterium freudenreichii subsp. freudenreichii can be suitably used.

In the present invention, the enzymes having a butylaldehyde dehydrogenase activity include:

(a) proteins having the amino acid sequence of SEQ ID NO: 1, 65, or 67;

(b) enzymes having an amino acid sequence in which one or more (2 or more, preferably 2 to 20, and more preferably 2 to 5) amino acids are substituted, deleted, inserted, or added in the amino acid sequence of SEQ ID NO: 1, 65, or 67;

(c) enzymes having an amino acid sequence encoded by the nucleotide sequence of SEQ ID NO: 2, 66, or 68;

(d) enzymes having an amino acid sequence encoded by a DNA which hybridizes under stringent conditions with a DNA comprising the nucleotide sequence of SEQ ID NO: 2, 66, or 68; and

(e) proteins having an identity of 85% or higher with the amino acid sequence of SEQ ID NO: 1, 65, or 67.

In the present invention, a homologue of an “enzyme comprising the amino acid sequence of a certain sequence identification number (SEQ ID NO)” can be rephrased as “an enzyme having an amino acid sequence in which one or more (2 or more, preferably 2 to 20, and more preferably 2 to 5) amino acids are substituted, deleted, inserted, or added in the amino acid sequence of a certain sequence identification number. This homologue refers to a protein that is functionally equivalent to an enzyme composed of the amino acid sequence of the specified sequence identification number. In the present invention, “functionally equivalent” means that a protein has the same enzymatic activity (catalytic reaction, chemical reaction, and such) of each enzyme described herein.

In the amino acid sequence of a certain sequence identification number, for example, 100 amino acid residues or less, usually 50 amino acid residues or less, preferably 30 amino acid residues or less, more preferably 15 amino acid residues or less, even more preferably 10 amino acid residues or less, or 5 amino acid residues or less may be mutated. Generally, in order to maintain the function of protein, an amino acid is preferably substituted with an amino acid having similar properties. Such amino acid substitution is referred to as conservative substitution. For example, Ala, Val, Leu, Ile, Pro, Met, Phe, and Trp are all classified as non-polar amino acids, and therefore possess similar properties. Uncharged amino acids include Gly, Ser, Thr, Cys, Tyr, Asn, and Gin. Acidic amino acids include Asp and Glu. Basic amino acids include Lys, Arg, and His. Substitution of amino acids within each group is acceptable.

A person skilled in the art can obtain a polynucleotide encoding a homologue of each enzyme by appropriately introducing substitution, deletion, insertion, and/or addition to the DNA of the enzyme which is composed of a nucleotide sequence disclosed herein, using methods such as site-specific mutagenesis (Nucleic Acid Res. 10, pp. 6487 (1982), Methods in Enzymol. 100, pp. 448 (1983), Molecular Cloning 2nd Edt., Cold Spring Harbor Laboratory Press (1989), PCR A Practical Approach IRL Press pp. 200 (1991)). The polynucleotide encoding a homologue of each enzyme can be introduced and expressed in a host to obtain the homologue.

Furthermore, in the present invention, a homologue of each enzyme refers to a protein which has an identity of at least 50%, preferably at least 70%, more preferably 80%, more preferably 85%, more preferably 90%, even more preferably 95% or higher (for example, 95%, 96%, 97%, 98%, or 99% or higher) with the amino acid sequence of SEQ ID NO corresponding to the enzyme. Protein homology searches can be carried out, for example, through the Internet by using a program such as BLAST and PASTA on amino acid sequence databases such as SWISS-PROT, PIR, and DAD, DNA sequence databases such as DDBJ, EMBL, and Genbank, databases of deduced amino acid sequences based on DNA sequences, or such.

Each of the enzymes described herein can be attached to an additional amino acid sequence as long as it retains an activity functionally equivalent to that of the enzyme. For example, a tag sequence such as histidine tag and HA tag can be added. Alternatively, the enzyme can be fused with another protein. Each enzyme of the present invention or a homologue thereof may be a fragment as long as it retains an activity functionally equivalent to that of the enzyme.

A polynucleotide encoding each of the enzymes described herein can be isolated by methods described below. For example, DNA of the present invention can be obtained by performing PCR using PCR primers designed based on the nucleotide sequence of SEQ ID NO corresponding to the enzyme, and using as a template a chromosomal DNA or a cDNA library from a strain producing the enzyme. Moreover, a polynucleotide of each enzyme can be obtained by performing colony hybridization or plaque hybridization using the obtained DNA fragment as a probe and using a cDNA library or a library obtained by transforming E. coli with phages or plasmids containing restriction enzyme digests of the chromosomal DNA of a strain producing the enzyme.

In addition, a polynucleotide of each enzyme can also be obtained as follows: The DNA, fragment obtained by PCR is sequenced, and the obtained nucleotide sequence is used to design PCR primers to extend outwardly from a known DNA. The chromosomal DNA of a strain producing the enzyme is digested by appropriate restriction enzymes, and DNAs resulting from self-circularization of the digests are used as templates to perform inverse PCR (Genetics 120, 621-623 (1988)). Alternatively, the RACE (Rapid Amplification of cDNA End) method (“PCR Experiment Manual”, p. 25-33, HBJ Publication Office) and such can also be performed.

In the present invention, the polynucleotides of each enzyme include genomic DNAs and cDNAs cloned by the above methods, and synthetically produced DNAs.

Hybridization is performed on a subject nucleic acid by using as a probe a nucleic acid (DNA or RNA) consisting of a complementary sequence or a partial sequence thereof of the nucleotide sequence of SEQ ID NO corresponding to each enzyme, and the presence or absence of significant hybridization between the probe and the subject nucleic acid is confirmed after washes under stringent conditions. The length of a probe used is, for example, consecutive 20 nucleotides or more, preferably 25 nucleotides or more, more preferably 30 nucleotides or more, more preferably 40 nucleotides or more, more preferably 80 nucleotides or more, more preferably 100 nucleotides or more (for example, a full length of the nucleotide sequence of SEQ ID NO corresponding to the enzyme). If a probe contains a sequence irrelevant to the nucleotide sequence of SEQ ID NO corresponding to the enzyme or its complementary sequence (such as a vector-derived sequence), this sequence can be used alone as a negative control probe to perform hybridization in the same manner, and the absence of significant hybridization between the probe and the subject sequence may be confirmed after washes under the same conditions. Hybridization can be carried out by common methods using nitrocellulose or nylon membranes (Sambrook et al. (1989) Molecular Cloning, Cold Spring Harbor Laboratories; Ausubel, F. M. et al. (1994) Current Protocols in Molecular Biology, Greene Publishe Associates/John Wiley and Sons, New York. NY).

Specific examples of stringent hybridization conditions are, for example, overnight hybridization in a solution containing 6×SSC, 0.5% (w/v) SDS, 100 μg/ml denatured salmon sperm DNA, and 5×Denhardt's solution (1×Denhardt's solution contains 0.2% polyvinylpyrrolidone, 0.2% bovine serum albumin, and 0.2% Ficoll) at 45° C., preferably 55° C., more preferably 60° C., and even more preferably 65° C., followed by three 20-min post-hybridization washes in 4×SSC and 0.5% SDS at the same temperature as hybridization. More preferably, post-hybridization washes are carried out with two 20-min washes in 4×SSC and 0.5% SDS followed by one 20-min wash in 2×SSC and 0.5% SDS, at the same temperature as hybridization. More preferably, post-hybridization washes are carried out with two 20-min washes in 4×SSC and 0.5% SDS, followed by one 20-min wash in 1×SSC and 0.5% SDS, at the same temperature as hybridization. More preferably, post-hybridization washes are carried out with one 20-min wash in 2×SSC and 0.5% SDS, followed by one 20-min wash in 1×SSC and 0.5% SDS, and one subsequent 20-min wash in 0.5×SSC and 0.5% SDS, at the same temperature as hybridization. More preferably, post-hybridization washes are carried out with one 20-min wash in 2×SSC and 0.5% SDS, followed by one 20-min wash in 1×SSC and 0.5% SDS, one 20-min wash in 0.5×SSC and 0.5% SDS, and one 20-min wash in 0.1×SSC and 0.5% SDS, at the same temperature as hybridization.

The activity of an enzyme having the function of EC1.2.1.10 which can be utilized in the present purposes can be confirmed, for example, as described below.

### Butylaldehyde Dehydrogenase (CoA-Acetylation) (BCDH) Activity Assay (Enzymatic Activity Shown by Formula 1)

A mixed solution of 100 mM Tris-HCl buffer (pH 6.5), 70 mM, semicarbazide (pH 6.5), 0.2 mM NADH, 0.2 mM 3-hydroxybutyryl-CoA or butyryl-CoA, and as necessary, 1 mM DTT, is equilibrated, at 30° C. for three minutes. A cell-free extract containing BCDH is added to the solution, and a decrease in absorbance at 340 nm associated with a decrease of NADH due to the reduction of acyl-CoA is measured. If an enzyme of interest may be inactivated in the presence of oxygen, the reaction solution is prepared and reacted under an anaerobic atmosphere (under a nitrogen atmosphere). One unit of an enzyme is defined as an amount of the enzyme which catalyzes a decrease of 1 μmol. NADH per minute under these conditions. Quantification of protein is carried out by a dye-binding assay using a protein assay kit manufactured by BioRad, and using bovine plasma albumin as a reference protein.

The present invention is related to recombinant microorganisms in which the enzymatic activity of (2) shown below is enhanced in addition to the enzymatic activity of (1) described above, and which produce 1,3-alkyldiol represented by Formula 2 from a fermentation substrate:

(1) activity of an enzyme that catalyzes production of 3-hydroxyalkylaldehyde by reducing 3-hydroxyacyl-CoA using NADH and/or NADPH as a coenzyme, as shown by Formula 1; and
(2) activity of an enzyme that catalyzes production of 1,3-alkyldiol represented by Formula 2 by reducing 3-hydroxyalkylaldehyde using NADH and/or NADPH as a coenzyme, as shown by Formula 3 (Formula 3 does not show two reactions, but only shows the production of alcohol from aldehyde).

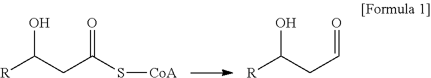

(wherein R represents C₁₋₃ alkyl or hydrogen; and CoA represents coenzyme A)

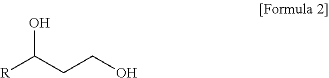

(wherein R represents C₁₋₃ alkyl or hydrogen)

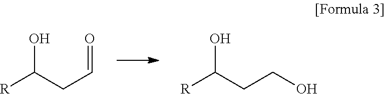

(wherein R represents C₁₋₃ alkyl or hydrogen)

Preferred diol compounds produced in the present invention include compounds of Formula 2 in Which R is a C₁₋₃ alkyl group (methyl group, ethyl group, propyl group, or isopropyl group) or hydrogen. More specifically, preferred examples of the diol compounds produced in the present invention include 1,3-butanediol (where R is methyl in Formulas 1 to 3). The optical activity of 1,3-butanediol produced in the present invention is not particularly limited, and the R-form and S-form of 1,3-butanediol ((R)- and (S)-1,3-butanediol) are included in 1,3-butanediol produced in the present invention.

In the present invention, the enzymes that catalyze the production of 1,3-alkyldiol represented by Formula 2 by reducing 3-hydroxyalkylaldehyde using NADH and/or NADPH as a coenzyme as shown by Formula 3 include enzymes classified as EC1.1.1.1, which have the systematic name of “alcohol: NAD⁺ oxidoreductase” given by the International Union of Biochemistry and Molecular Biology (IUBMB) (http://www.chem.qmul.ac.uk/iubmb/), and catalyze the following reaction: alcohol+NAD⁺=aldehyde or keton+NADH+H⁺. Specifically, the above enzymes include butanol dehydrogenase derived from Clostridium acetobutylicum.

In the present invention, the enzymes having a butanol dehydrogenase activity include:

(a) proteins having the amino acid sequence of SEQ ID NO: 3, 5, or 7;

(b) enzymes having an amino acid sequence in which one or more amino acids are substituted, deleted, inserted, or added in the amino acid sequence of SEQ ID NO: 3, 5, or 7;

(c) enzymes having an amino acid sequence encoded by the nucleotide sequence of SEQ ID NO: 4, 6, or 8;

(d) enzymes having an amino acid sequence encoded by a DNA which hybridizes under stringent conditions with a DNA comprising the nucleotide sequence of SEQ ID NO: 4, 6, or 8; and

(e) enzymes having an identity of 85% or higher with the amino acid sequence of SEQ ID NO: 3, 5, or 7.

The activity of an enzyme having the function of EC1.1.1.1 which can be utilized in the present purposes can be confirmed, for example, as described below.

### Butanol Dehydrogenase (BDH) Activity Assay (Enzymatic Activity Shown by Formula 2)

A mixed solution of 50 mM MES buffer (pH 6.0), 0.2 mM NADH, 20 mM 3-hydroxybutylaldehyde or butylaldehyde, and as necessary, 1 mM. DTT, is equilibrated at 30° C. for three minutes. A cell-free extract containing BDH is added to the solution, and a decrease in absorbance at 340 nm associated with a decrease of NADH due to the reduction of alkylaldehyde is measured. If an enzyme of interest may be inactivated in the presence of oxygen, the reaction solution is prepared and reacted under an anaerobic atmosphere (under a nitrogen atmosphere). One unit of an enzyme is defined as an amount of the enzyme which catalyzes a decrease of 1 μmol NADH per minute. Quantification of protein is carried out by a dye-binding assay using a protein assay kit manufactured by BioRad, and using bovine plasma albumin as a reference protein.

The present invention is related to recombinant microorganisms in which the enzymatic activity of (3) shown below is enhanced in addition to the enzymatic activity of (1) described above, and which produce 1,3-alkyldiol represented by Formula 2 from a fermentation substrate:

(1) activity of an enzyme that catalyzes production of 3-hydroxyalkylaldehyde by reducing 3-hydroxyacyl-CoA using NADH and/or NADPH as a coenzyme, as shown by Formula 1; and
(3) activity of an enzyme that produces 3-hydroxyacyl-CoA by reducing 3-oxoacyl-CoA in an NADH- and/or NADPH-dependent manner as shown by Formula 4.

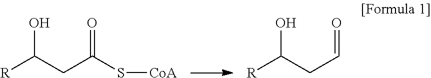

(wherein R represents C₁₋₃ alkyl or hydrogen; and CoA represents coenzyme A)

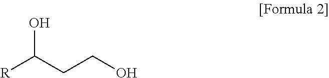

(wherein R represents C₁₋₃ alkyl or hydrogen)

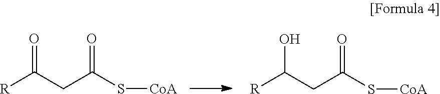

(wherein R represents C₁₋₃ alkyl or hydrogen; and CoA represents coenzyme A)

Preferred diol compounds produced in the present invention include compounds of Formula 2 in which R is a C₁₋₃ alkyl group (methyl group, ethyl group, propyl group, or isopropyl group) or hydrogen. More specifically, preferred examples of the diol compounds produced in the present invention include 1,3-butanediol (where R is methyl in Formulas 1, 2, and 4). The optical activity of 1,3-butanediol produced in the present invention is not particularly limited, and the R-form and S-form of 1,3-butanediol ((R)- and (S)-1,3-butanediol) are included in 1,3-butanediol produced in the present invention.

In the present invention, any enzymes capable of catalyzing the reaction of producing 3-hydroxybutyryl-CoA by reducing the 3-carbonyl group of acetoacetyl-CoA can be used for the reaction of producing 3-hydroxyacyl-CoA by reducing 3-oxoacyl-CoA as shown by Formula 4 (for example, when R is methyl, the reaction of producing 3-hydroxybutyryl-CoA from acetoacetyl-CoA). Preferably, such enzymes are usually possessed by microorganisms having a poly(3-hydroxybutanoic acid) (PHB) synthesis pathway or a butanol fermentation pathway. Microorganisms having a PHB synthesis pathway include Ralstonia eutropha and Zoogloea ramigera, and the acetoacetyl-CoA reductases (whose genes are generally denoted as phaB or phbB) of such microorganisms are examples of preferred enzymes. Microorganisms having a butanol fermentation pathway include Clostridium bacteria, known as acetone-butanol fermentation bacteria as mentioned above, and the 3-hydroxybutyryl-CoA dehydrogenases (whose genes are generally denoted as hbd) of such microorganisms are included. Moreover, from the viewpoint of enzyme-substrate specificity, enzymes catalyzing a similar reaction can also be used. For example, β-ketoacyl-ACP reductases in fatty acid synthesis pathways, specifically, β-ketoacyl-ACP reductase (BstKR1) derived from Bacillus stearothermophilus, can be used. In addition, β-ketoacyl reductase derived from Streptomyces violaceoruber (SvKR1), an enzyme in the polyketide actinorhodin synthesis pathway, can also be used. As other examples, carbonyl reductases that reduce β-ketocarboxylic acid or the 3-carbonyl group of its ester can be used. More specifically, (R)-2-octanol dehydrogenase derived from Pichia finlandica (PfODH) can be used. If these reductases have high stereoselectivity, they are suitable for obtaining optically active 1,3-butanediol. To obtain (R)-1,3-butanediol, acetoacetyl-CoA reductase derived from Ralstonia eutropha (ReAR1), acetoacetyl-CoA reductase derived from Zoogloea ramigera (ZrAR1), BstKR1, SvKR1, and PfODH, which are highly (R)-selective, and more preferably, ReAR1, can be used as suitable enzymes for producing (R)-1,3-butanediol. To obtain (S)-1,3-butanediol, HBD derived from Clostridium, which is highly (S)-selective, and more preferably, 3-hydroxybutyryl-CoA dehydrogenase derived from Clostridium acetobutylicum (CaHBD), can be used as suitable enzymes for producing (S)-1,3-butanediol. These compounds are preferred optically-active alcohols that can be produced by the present invention.

Of the enzymes producing 3-hydroxyacyl-CoA by reducing 3-oxoacyl-CoA in the present invention (for example, when R is methyl, the reaction of producing 3-hydroxybutyryl-CoA from acetoacetyl-CoA), preferred enzymes for producing (R)-1,3-butanediol as a final product include:

(a) proteins having the amino acid sequence of SEQ ID NO: 9, 11, 13, 15, or 17;

(b) enzymes having an amino acid sequence in which one or more amino acids are substituted, deleted, inserted, or added in the amino acid sequence of SEQ ID NO: 9, 11, 13, 15, or 17;

(c) enzymes having an amino acid sequence encoded by the nucleotide sequence of SEQ ID NO: 10, 12, 14, 16, or 18;

(d) enzymes having an amino acid sequence encoded by a DNA which hybridizes under stringent conditions with a DNA comprising the nucleotide sequence of SEQ ID NO: 10, 12, 14, 16, or 18; and

(e) enzymes having an identity of 85% or higher with the amino acid sequence of SEQ ID NO: 9, 11, 13, 15, or 17.

Of the enzymes producing 3-hydroxyacyl-CoA by reducing 3-oxoacyl-CoA in the present invention (for example, when R is methyl, the reaction of producing 3-hydroxybutyryl-CoA from acetoacetyl-CoA), preferred enzymes for producing (s)-1,3-butanediol as a final product include:

(a) proteins having the amino acid sequence of SEQ ID NO: 19;

(b) enzymes having an amino acid sequence in which one or more amino acids are substituted, deleted, inserted, or added in the amino acid sequence of SEQ ID NO: 19;

(c) enzymes having an amino acid sequence encoded by the nucleotide sequence of SEQ ID NO: 20;

(d) enzymes having an amino acid sequence encoded by a DNA which hybridizes under stringent conditions with a DNA comprising the nucleotide sequence of SEQ ID NO: 20; and

(e) enzymes having an identity of 85% or higher with the amino acid sequence of SEQ ID NO: 19.

The activity of an enzyme reducing the 3-carbonyl group of 3-hydroxybutyryl-CoA which can be utilized in the present purposes can be confirmed, for example, as described below.

### 3-Hydroxybutyryl-CoA Dehydrogenase (3HBD) Activity Assay

A mixed solution of 100 mM potassium phosphate buffer (pH 6.5), 0.2 mM NAD(P)H, 0.2 mM acetoacetyl-CoA, and as necessary, 1 mM DTT, is equilibrated at 30° C. for three minutes. A cell-free extract containing 3HBD is added to the solution, and a decrease in absorbance at 340 nm associated with a decrease of NAD(P)H due to the reduction of acetoacetyl-CoA is measured. One unit of an enzyme is defined as an amount of the enzyme which catalyzes a decrease of 1 μmol NAD(P)H per minute. Quantification of protein is carried out by a dye-binding assay using a protein assay kit manufactured by BioRad, and using bovine plasma albumin as a reference protein.

The present invention is related to recombinant microorganisms in which the enzymatic activity of (4) shown below is enhanced in addition to the enzymatic activity of (1), and which produce (R)- or (S)-1,3-butanediol represented by Formula 2 from a fermentation substrate:

(1) activity of an enzyme that catalyzes production of 3-hydroxybutylaldehyde by reducing β-hydroxybutyryl-CoA using NADH and/or NADPH as a coenzyme, as shown by Formula 1; and
(4) activity of a β-ketothiolase that catalyzes production of acetoacetyl-CoA from two molecules of acetyl-CoA, as shown by Formula 7.

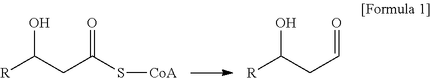

(wherein R represents methyl; and CoA represents coenzyme A)

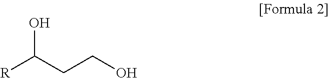

(wherein R represents methyl)

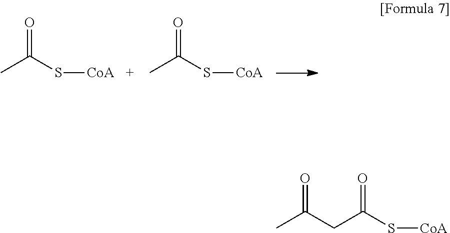

(wherein CoA represents coenzyme A)

Preferred diol compounds produced in the present invention include 1,3-butanediol (where R is methyl in Formulas 1, 2, and 7). The optical activity of 1,3-butanediol produced in the present invention is not particularly limited, and the R-form and S-form of 1,3-butanediol ((R)- and (S)-1,3-butanediol) are included in 1,3-butanediol produced in the present invention.

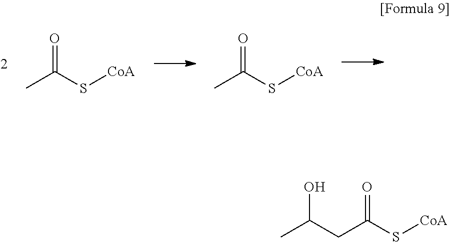

Of the reactions shown by Formula 9, the reaction from acetyl-CoA to acetoacetyl-CoA (the reaction of Formula 7) can be performed using any enzyme capable of producing acetoacetyl-CoA by condensing two molecules of acetyl-CoA. Preferred enzymes include enzymes classified as EC2.3.1.9, which have the systematic name of “acetyl-CoA:acetyl-CoA C-acetyltransferase” given by IUBMB and catalyze the following reaction: 2 acetyl-CoA CoA acetoacetyl-CoA; and enzymes classified as EC2.3.1.16, which have the systematic name of “acyl-CoA:acetyl-CoA C-acyltransferase” and catalyze the following reaction: acyl-CoA acetyl-CoA=CoA+3-oxoacyl-CoA (http://www.chem.qmul.ac.uk/iubmb/).

Specifically, enzymes of poly(3-hydroxybutanoic acid) (PHB) synthesis pathways, butanol fermentation pathways, and such, which are possessed by microorganisms and the like, can be usually used. More specifically, microorganisms having a PHB synthesis pathway include Ralstonia eutropha and Zoogloea ramigera, and acetyl-CoA acetyltransferases or β-ketothiolase of such microorganisms (whose genes are generally denoted as phaA and phbA) are examples of preferred enzymes. In addition, microorganisms having a butanol fermentation pathway include Clostridium bacteria, which are known as acetone-butanol fermentation bacteria as described above, and acetyl-CoA acetyltransferases or β-ketothiolases of such microorganisms (whose genes are generally denoted as thl and thi) are examples of preferred enzymes. Other examples include the product of a gene generally denoted as atoB, which is produced by Escherichia coli and such. Moreover, β-ketoacyl-ACP synthases of fatty acid biosynthesis systems, which are typically possessed by microorganisms, can also be used.

In the present invention, the enzymes having an acetyl-CoA acetyltransferase activity include:

(a) proteins having the amino acid sequence of SEQ ID NO: 21, 23, or 25;

(b) enzymes having an amino acid sequence in which one or more amino acids are substituted, deleted, inserted, or added in the amino acid sequence of SEQ ID NO: 21, 23, or 25;

(c) enzymes having an amino acid sequence encoded by the nucleotide sequence of SEQ ID NO: 22, 24, or 26;

(d) enzymes having an amino acid sequence encoded by a DNA which hybridizes under stringent conditions with a DNA comprising the nucleotide sequence of SEQ ID NO: 22, 24, or 26; and

(e) enzymes having an identity of 85% or higher with the amino acid sequence of SEQ ID NO: 21, 23, or 25.

The activity of an acetyl-CoA acetyltransferase (or β-ketothiolase) which can be utilized in the present purposes can be confirmed, for example, as described below.

### β-Ketothiolase (THL) Activity Assay-1

A mixed solution of 100 mM Tris-HCl buffer (pH 8.0), 10 mM magnesium chloride, 0.2 mM CoA, 0.05 mM acetoacetyl-CoA, and as necessary, 1 mM DTT, is equilibrated at 30° C. for three minutes. A cell-free extract containing β-ketothiolase is added to the solution, and degradation of Mg²⁺-acetoacetyl-CoA complex is measured as a decrease in absorbance at 303 nm. One unit of an enzyme is defined as an amount of the enzyme which catalyzes a decrease of 1 mol acetoacetyl-CoA per minute. Quantification of protein is carried out by a dye-binding assay using a protein assay kit manufactured by BioRad, and using bovine plasma albumin as a reference protein.

### β-Ketothiolase (THL) Activity Assay-2

A mixed solution of 100 mM Tris-HCl buffer (pH 7.5), 2.0 mM NADH, 0.2 mM acetyl-CoA, and 2.0 U of 3-hydroxybutyryl-CoA dehydrogenase derived from Clostridium acetobutylicum, is equilibrated at 30° C. for three minutes. A cell-free extract containing β-ketothiolase is added to the solution, and a decrease in absorbance at 340 nm associated with a decrease of NADH during the reduction of acetoacetyl-CoA following the condensation of acetyl-CoA, is measured. One unit of an enzyme is defined as an amount of the enzyme which catalyzes a decrease of 1 μmol NADH per minute. Quantification of protein is carried out by a dye-binding assay using a protein assay kit manufactured by BioRad, and using bovine plasma albumin as a reference protein.

The present invention is related to recombinant microorganisms in which the enzymatic activities of (2) to (4) shown below are enhanced in addition to the enzymatic activity of (1) shown below, and which produce (R)- or (S)-1,3-butanediol represented by Formula 2 from a fermentation substrate:

(1) activity of an enzyme that catalyzes production of 3-hydroxybutylaldehyde by reducing β-hydroxybutyryl-CoA using NADH and/or NADPH as a coenzyme, as shown by Formula 1;
(2) activity of an enzyme that catalyzes production of 1,3-butanediol represented by Formula 2 by reducing 3-hydroxybutylaldehyde using NADH and/or NADPH as a coenzyme, as shown by Formula 3 (Formula 3 does not show two reactions, but only shows the production of alcohol from aldehyde);
(3) activity of an enzyme that produces 3-hydroxybutyryl-CoA by reducing acetoacetyl-CoA in an NADH- and/or NADPH-dependent manner as shown by Formula 4; and
(4) activity of a β-ketothiolase that catalyzes production of acetoacetyl-CoA from two molecules of acetyl-CoA as shown by Formula 7.

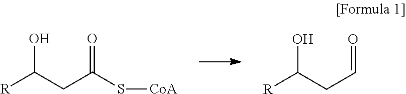

(wherein R represents methyl; and CoA represents coenzyme A)

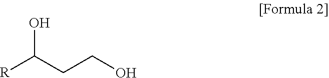

(wherein R represents methyl)

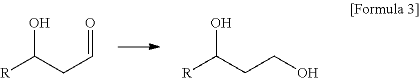

(wherein R represents methyl)

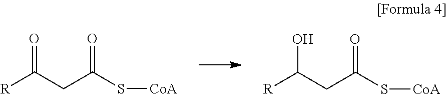

(wherein R represents methyl; and CoA represents coenzyme A)

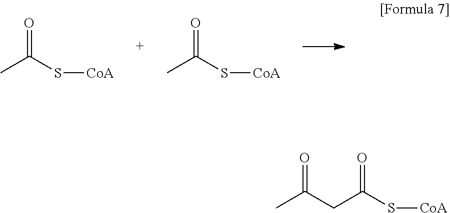

(wherein CoA represents coenzyme A)

In each of the above enzymatic reactions, the above-described enzymes suitable for each step can be used.

For the recombinant microorganisms of the present invention, host cells are not particularly limited, but more preferably, Escherichia coli can be used as a host cell.

In the present invention, the phrase “activity is enhanced” refers to a recombinant microorganism that is produced by introducing into a host a gene of interest that is derived from the same or different species and is not possessed by or is expressed at an extremely low level by the host, such that the recombinant microorganism has twice or more, preferably three times or more, more preferably five times or more, even more preferably 10 times or more the activity of the host.

In the present invention, an “optically active alcohol” refers to an alcohol containing a greater amount of one of its optical isomers than the other optical isomers. In the present invention, preferred optically active amine derivatives have, for example, 60%, typically 70% or higher, preferably 80% or higher, and still more preferably 90% or higher enantiomeric excess (ee). The “optical isomers” of the present invention may also be generally referred to as “enantiomers”.

Polynucleotides encoding the enzymes of the present invention (for example, enzymes having the function of EC1.2.1.10) can be isolated by methods as described below.

DNAs of the present invention can be obtained by designing PCR primers based on a known nucleotide sequence corresponding to each enzyme, and then performing PCR by using as a template a chromosomal DNA or a cDNA library obtained from a strain producing the enzyme.

Furthermore, polynucleotides of the present invention can be obtained by performing colony hybridization or plaque hybridization using the obtained DNA fragment as a probe and using a cDNA library or a library obtained by transforming E. coli with phages or plasmids containing restriction enzyme digests of the chromosomal DNA of an enzyme-producing strain.

In addition, polynucleotides of the present invention can also be obtained as follows: The DNA fragment obtained by PCR is sequenced, and the obtained nucleotide sequence is used to design PCR primers to extend outwardly from a known DNA. The chromosomal DNA of an enzyme-producing strain is digested by appropriate restriction enzymes, and DNAs resulting from self-circularization of the digests are used as templates to perform inverse PCR (Genetics 120, 621-623 (1988)). Alternatively, the RACE (Rapid Amplification of cDNA End) method (“PCR Experiment Manual”, p. 25-33, HBJ Publication. Office) and such can also be performed.

The polynucleotides of the present invention include genomic DNAs and cDNAs cloned by the above methods, and synthetically produced DNAs.

Expression vectors for the enzymes of the present invention (for example, enzymes having the function of EC1.2.1.10) are provided by introducing a polynucleotide encoding each enzyme isolated as described above into known expression vectors. Preferably, a polynucleotide encoding the above enzyme and a polynucleotide(s) encoding acetyl-CoA-acetyltransferase (or β-ketothiolase) and/or stereoselective 3-hydroxybutyryl-CoA dehydrogenase and/or butanol dehydrogenase as obtained by the same methods described above are inserted into a known vector at the same time.

In the present invention, microorganisms subjected to transformation at least for expressing each enzyme (for example, an enzyme having the function of EC1.2.1.10) are not particularly limited as long as they can be transformed with a recombinant vector containing a polynucleotide encoding a polypeptide having the enzyme (for example, an enzyme having the function of EC1.2.1.10), and can express the activity of the enzyme (for example, an enzyme having the function of EC1.2, 1.10). Microorganisms that can be used include, for example:

### Escherichia;

### Bacillus;

### Pseudomonas;

### Serratia;

### Brevibacterium;

### Corynebacterium;

### Streptococcus;

Bacteria for which host-vector systems have been developed, such as Lactobacillus;

### Rhodococcus;

Actinomycetes for which host-vector systems have been developed, such as Streptomyces;

### Saccharomyces;

### Kluyveromyces;

### Schizosaccharomyces;

### Zygosaccharomyces;

### Yarrowia;

### Trichosporon;

### Rhodosporidium;

### Pichia;

Yeasts for which host-vector systems have been developed, such as Candida;

### Neurospora;

### Aspergillus;

### Cephalosporium; and

Fungi for which host-vector systems have been developed, such as Trichoderma.

Preparation of transformants and construction of recombinant vectors adapted to hosts can be carried out according to techniques commonly used in the fields of molecular biology, bioengineering, and genetic engineering (for example, Sambrook et al., Molecular Cloning, Cold Spring Harbor Laboratories).

In order to express a gene of each enzyme of the present invention (for example, an enzyme having the function of EC1.2.1.10) in microbial cells or such, DNA of the present invention is first introduced into a plasmid or phage vector that can be stably present within a microorganism and allow the organism to transcribe and translate its genetic information. To achieve this, a promoter, which is a regulatory unit for transcription/translation, is inserted 5′-upstream of the DNA strand of the present invention, and more preferably, a terminator is inserted 3′-downstream of the DNA. Any promoters or terminators known to be functional in a microorganism to be used as a host are used. Vectors, promoters, terminators, and such that can be used in various microorganisms are described in detail in, for example, “Fundamental Microbiology (Biseibutsugaku Kiso-kouza) 8: Genetic Engineering, KYORITSU SHUPPAN CO., LTD.”. In particular, those to be used with yeasts are described in detail in Adv. Biochem. Eng. 43, 75-102 (1990); Yeast 8, 423-488 (1992); and such. For the genus Escherichia, in particular Escherichia coli, available plasmid vectors include the pBR series and pUC series plasmids. Available promoters include promoters derived from lac (β-galactosidase), trp (tryptophan operon), tac and trc (fusion of lac and trp), and PL and PR of λ phage. Available terminators are those derived from trpA, phages, rmB ribosomal RNA, etc.

For the genus Bacillus, available vectors include pUB110 series and pC194 series plasmids, and they can be integrated into a chromosome. Available promoters and terminators are those derived from apr (alkaline protease), npr (neutral protease), amy (α-amylase), etc.

For the genus Pseudomonas, host-vector systems have been developed for Pseudomonas putida, Pseudomonas cepacia, and such. It is possible to use wide host-range vectors such as pKT240 (containing genes required for autonomous replication derived from RSF1010 and such) constructed based on the TOL plasmid, which is involved in the degradation of toluene compounds. Available promoters and terminators include those from the lipase gene (JP-A (Kokai) H05-284973).

For the genus Brevibacterium, in particular Brevibacterium lactofermentum, it is possible to use plasmid vectors such as pAJ43 (Gene 39, 281 (1985)). Promoters and terminators used for E. coli can be used without modification.

For the genus Corynebacterium, in particular Corynebacterium glutamicum, it is possible to use plasmid vectors such as pCS11 (JP-A (Kokai) S57-183799) and pCB101 Gen. Genet. 196, 175 (1984)).

For the genus Streptococcus, it is possible to use plasmid vectors such as pHV1301 (FEMS Microbiol. Lett. 26, 239 (1985)) and pGK1 (Appl. Environ. Microbiol. 50, 94 (1985)).

For the genus Lactobacillus, it is possible to use pAMβ1 (J. Bacteriol. 137, 614 (1979)), which has been developed for Streptococcus, and such. Promoters used for E. coli can be used.

For the genus Rhodococcus, it is possible to use plasmid vectors and such isolated from Rhodococcus rhodochrous (J. Gen. Microbiol. 138, 1003 (1992)).

For the genus Streptomyces, plasmids can be constructed by the method described in Hopwood et al., Genetic Manipulation of Streptomyces: A Laboratory Manual, Cold Spring Harbor Laboratories (1985). In particular, for Streptomyces lividans, it is possible to use pIJ486 (Mol. Gen. Genet. 203, 468.478, 1986), pKC1064 (Gene 103, 97-99 (1991)), pUWL-KS (Gene 165, 149-150 (1995)), and such. The same plasmids can also be used for Streptomyces virginiae (Actinomycetol. 11, 46-53 (1997)).

For the genus Saccharomyces, in particular Saccharomyces cerevisiae, it is possible to use YRp series plasmids, YEp series plasmids, YCp series plasmids, Ylp series plasmids and such. Integration vectors that can be homologously recombined with ribosomal DNA (e.g. EP 537456), multiple copies of which are present within chromosomes, are highly useful since they allow multiple copies of a gene to be introduced and stably maintained. In addition, promoters and terminators such as those derived from ADH (alcohol dehydrogenase), GAPDH (glyceraldehyde-3-phosphate dehydrogenase), PHO (acid phosphatase), GAL (β-galactosidase), PGK (phosphoglycerate kinase), and ENO (enolase) can be used.

For the genus Kluyveromyces, in particular Kluyveromyces lactis, it is possible to use Saccharomyces cerevisiae-derived 2 μm plasmids, pKD1 series plasmids (J. Bacteriol. 145, 382-390 (1981)), plasmids derived from pGK11 which is involved in the killer activity, plasmids based on KARS which is an autonomously replicating gene in Kluyveromyces, vector plasmids that can be integrated into chromosomes by homologous recombination with ribosome DNA or such (e.g. EP 537456), and the like. Promoters and terminators derived from ADH, PGK, and such can also be used.

For the genus Schizosaccharomyces, it is possible to use ARS (a gene involved in autonomous replication) derived from Schizosaccharomyces pombe, plasmid vectors derived from Saccharomyces cerevisiae that contain an auxotrophy-complementing selection marker (Mol. Cell. Biol. 6, 80 (1986)), and such. In addition, the ADH promoter derived from Schizosaccharomyces pombe and such can be used (EMBO J. 6, 729 (1987)). In particular, pAUR224 is commercially available from Takara and can be readily used.

For the genus Zygosaccharomyces, it is possible to use plasmid vectors derived from pSB3 derived from Zygosaccharomyces rouxii (Nucleic Acids Res. 1.3, 4267 (1985)), and such. The PHO5 promoter derived from Saccharomyces cerevisiae, the promoter of GAP-Zr (glyceraldehyde-3-phosphate dehydrogenase) (Agri. Biol. Chem. 54, 2521 (1990)), and such can be used.

For the genus Pichia, host-vector systems have been developed for Pichia angusta (previously called Hansenula polymorpha). Although Pichia angusta-derived genes involved in autonomous replication (HARS1 and HARS2) are available as vectors, they are rather unstable and thus multicopy chromosomal integration is effective (Yeast 7, 431-443 (1991)). In addition, methanol-inducible promoters of alcohol oxidase (AOX) and formate dehydrogenase (FDH) and the like are available. Furthermore, host-vector systems based on Pichia-derived genes involved in autonomous replication (PARS1, PARS2) have been developed (Mol. Cell. Biol., 5, 3376 (1985)). Strong promoters such as AOX promoter, which is inducible by high-cell-density-culture and methanol, can also be employed (Nucleic Acids Res. 15, 3859 (1987)).

In the genus Candida, host-vector systems have been developed for Candida maltosa, Candida albicans, Candida tropicalis, Candida utilis, etc. An ARS originating from Candida maltosa has been cloned (Agri. Biol. Chem. 51, 51, 1587 (1987)), and a vector using this sequence has been developed thr Candida maltosa. Furthermore, a strong promoter for a chromosomal integration vector has been developed for Candida utilis (JP-A (Kokai) Hei 08-173170).

For the genus Aspergillus, Aspergillus niger and Aspergillus oryzae are the most studied fungi, and plasmid vectors and chromosome-integration vectors are available. Promoters derived from an extracellular protease gene and amylase gene can be used (Trends in Biotechnology 7, 283-287 (1989)).

For the genus Trichoderma, host-vector systems have been developed for Trichoderma reesei, and promoters such as those derived from extracellular cellulase genes are available (Biotechnology 7, 596-603 (1989)).

Various host-vector systems have also been developed for plants and animals. In particular, systems for expressing a large amount of foreign protein in insects such as silkworm (Nature 315, 592-594 (1985)), and plants such as rapeseed, maize, and potato, have been developed and they can be suitably used.

The present invention is related to methods for producing 1,3-butanediol using a recombinant microorganism in which the above-mentioned enzymatic activity(ies) is enhanced, and which produces an alcohol compound represented by Formula 2. More specifically, the present invention is related to methods for producing a diol compound represented by Formula 2, comprising the steps of contacting a fermentation substrate with at least one active material selected from the group consisting of a culture of a recombinant microorganism which functionally expresses the above enzymatic activity(ies), a cell of the microorganism, and a processed product thereof, and collecting 1,3-alkyldiol represented by Formula 2.

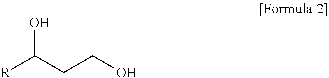

(wherein R represents C₁₋₃ alkyl or hydrogen)

Preferred diol compounds produced in the present invention include compounds of Formula 2 in which, for example, R is a C₁₋₃ alkyl group (methyl group, ethyl group, propyl group, or isopropyl group) or hydrogen. More specifically, preferred examples of the diol compounds produced in the present invention include 1,3-butanediol (where R is methyl in Formulas 1, 2, and 4). The optical activity of 1,3-butanediol produced in the present invention is not particularly limited, and the R-form and S-form of 1,3-butanediol ((R)- and (S)-1,3-butanediol) are included in 1,3-butanediol produced in the present invention.

Production of 1,3-butanediol can be carried out by contacting a fermentation substrate with, for example, a recombinant microorganism that functionally expresses the above enzyme(s), thereby allowing the recombinant microorganism to assimilate the fermentation substrate and perform a desired enzymatic reaction(s). The mode of contact between the recombinant microorganism and the fermentation substrate is not limited to these specific examples. The fermentation substrate is dissolved in a suitable solvent to provide a desirable environment for the recombinant microorganism to assimilate the fermentation substrate and express the desired enzymatic activity.

Preferred microorganisms used in the above methods include transformants that functionally express an enzyme(s) suitable for the above-mentioned step(s).

In the present invention, processed products of a transformant in which the enzymatic activity catalyzing the reduction reaction of Formula 1 is enhanced include, specifically, a microorganism with cellular membrane permeability altered by treatment with surfactant or organic solvent such as toluene; dried cells prepared by lyophilization or spray drying; a cell-free extract obtained by disrupting cells with glass beads or by enzymatic treatment, or a partially-purified product thereof; a purified enzyme; and an immobilized enzyme or immobilized microorganism.

Fermentation substrates used as a raw material in the methods for producing 1,3-butanediol in the present invention include sugars such as glucose, lactose, xylose, and sucrose. In addition, depending on the host microorganism used, it is possible to use any substrates such as glycerol and CO₂ which can be catabolized by the microorganism into acetyl-CoA and/or acetoacetyl-CoA and/or 3-hydroxybutyryl-CoA.

3-Hydroxybutylaldehyde, a substrate used in the activity assay for enzymes having only the function of EC1.1.1.1 according to the present invention, can be synthesized by, for example, reacting two equivalents of acetaldehyde in an ether solvent.

Furthermore, 1,3-butanediol can be produced more efficiently by simultaneously introducing an acetyl-CoA acetyltransferase (or β-ketothiolase) gene and/or 3-hydroxybutyryl-CoA dehydrogenase gene into a transformant in which the enzymatic activity catalyzing the reduction reaction of Formula 1 is enhanced. Moreover, butyrylaldehyde dehydrogenase and/or butanol dehydrogenase genes may be introduced simultaneously. Two or more of these genes can be introduced into a host by methods such as the following: transforming the host with genes that have been separately inserted into multiple recombinant vectors having different replication origins in order to avoid incompatibility; introducing all genes into a single vector; and introducing one or more genes into chromosomes.

When multiple genes are introduced into a single vector, each gene can be ligated to expression regulatory regions such as a promoter and terminator. Multiple genes can also be expressed as a polycistronic operon like the lactose operon.

When 1,3-butanediol is produced by contacting a fermentation substrate with a transformant of the present invention in which the enzymatic activity catalyzing the reduction reaction of Formula 1 is enhanced, or with a processed product thereof, it is possible to select conditions preferred for the activity and stability of the enzyme catalyzing the reduction reaction of Formula 1, and/or for the activity of the transformant to assimilate the fermentation substrate.

The concentration of a fermentation substrate used as a raw material for producing 1,3-butanediol is not particularly limited, but is usually about 0.1-30%, preferably 0.5-15%, and more preferably 1-10%.

In the present invention, “%” always means “weight/volume (w/v)”. In the case of (R)-1,3-butanediol, “%e.e.” refers to a value calculated by (([concentration of (R)-1,3-butanediol]−[concentration of (S)-1,3-butanediol])/([concentration of (R)-1,3-butanediol]+[concentration of (S)-1,3-butanediol]))×100. Likewise, in the case of (S)-1,3-butanediol, “%e.e.” refers to a value calculated by (([concentration of (S)-1,3-butanediol]−[concentration of (R)-1,3-butanediol])/([concentration of (S)-1,3-butanediol]+[concentration of (R)-1,3-butanediol]))×100.

A raw material may be added all at once when starting fermentation, but may also be added continuously or intermittently to the fermentation liquid.

As a recombinant microorganism of the present invention, preferably, it is possible to use recombinant E. coli prepared by isolating a gene of an enzyme catalyzing the reduction reaction of Formula 1 and enhancing its activity in host E. coli. Recombinant E. coli used for the present purposes can be cultured in media that are commonly used to culture E. coli, and induced to overexpress the gene by known methods. For example, E. coli cells in which the above-mentioned enzymatic activity is enhanced are cultured in 2×YT medium (2.0% Bacto-tryptone, 1.0% Bacto-yeast extract, 1.0% sodium chloride, pH 7.2), and are induced to express the enzyme by isopropyl-thio-β-D-galactopyranoside (IPTG). After the cells are proliferated sufficiently, the culture itself or cells harvested therefrom can be used for 1,3-butanediol production.

Recombinant microorganisms used for 1,3-butanediol production in the present invention may be allowed to produce 1,3-butanediol while being grown in a fermentation liquid for 1,3-butanediol production which contains a fermentation substrate as described below. It is possible to use a previously-grown culture or harvested cells. The amount of a previously-grown culture or harvested cells is usually about 0.1%-100%, preferably 0.5%-50%, and more preferably 1-20%, relative to the amount of the fermentation liquid for 1,3-butanediol production containing a fermentation substrate (“%” mentioned here refers to the ratio of inoculated cells to the fermentation liquid for 1,3-butanediol production, which is represented by [previously-gown culture]/[fermentation liquid for 1,3-butanediol production] (v/v)).

In addition to a fermentation substrate for 1,3-butanedial production, the fermentation liquid for 1,3-butanediol production preferably contains, as necessary, materials that promote the production of 1,3-butanediol. It may contain culture medium components that serve as nutrients for recombinant E. coli used for the present purposes. Specifically, such components include those of media used for culturing E. coli such as LB (1.0% Bacto-tryptone, 0.5% Bacto-yeast extract, 1.0% sodium chloride, pH 7.2), 2×YT (2.0% Bacto-tryptone, 1.0% Bacto-yeast extract, 1.0% sodium chloride, pH 7.2), and M9 medium (6.8 g/L Na₂HPO₄, 3.0 g/L KH₂PO₄, 0.5 g/L NaCl, 1.0 g/L NH₄Cl, 0.493 g/L MgSO₄.7H₂O, 14.7 mg/L CaCl₂.2H₂O, pH7.5). When a sufficient amount of previously-grown cells are provided in the fermentation liquid for 1,3-butanediol production, 1,3-butanediol can be produced in the presence of a higher concentration of a fermentation substrate, and it is also possible to remove the above-mentioned media. In addition, the fermentation liquid may also contain a component to maintain pH suitable for the 1,3-butanediol production during fermentation, such as a buffer agent at a concentration of 10 mM to 800 mM, preferably 50 mM to 500 mM, and more preferably 100 mM to 250 mM. Specifically, such buffer agents include MOPS buffer, HEPES buffer, MES buffer, Tris buffer, and phosphate buffer. Fermentation may be performed at any temperature that allows a recombinant microorganism of the present invention to exhibit its ability to assimilate a fermentation substrate, express the enzymatic activity to catalyze the reduction reaction of Formula 1, and thereby produce 1,3-butanediol. Such temperature may be usually in the range of 5° C. to 60° C., preferably 10° C. to 50° C., and more preferably 20° C. to 40° C. Also, fermentation may be performed at any pH that allows the expression of the enzymatic activity catalyzing the reduction reaction of Formula 1 and the production of 1,3-butanediol. Such pH may be usually in the range of pH 4 to 12, preferably pH 5 to 11, and more preferably pH 6 to 9. Fermentation can be performed while stirring or standing still. In addition, in order to efficiently convert a fermentation substrate into 1,3-butanediol, fermentation can be performed in a reaction medium under aerobic conditions in which a sufficient amount of oxygen is supplied, under microaerobic conditions in which the supply of oxygen is limited, or under anaerobic conditions in which no oxygen is supplied.

The production of 1,3-butanediol in the present invention can be performed in water, in a water-insoluble organic solvent such as ethyl acetate, butyl acetate, toluene, chloroform, n-hexane, methyl isobutyl ketone, methyl tertiary butyl ether, and diisopropyl ether; in a two-phase system with an aqueous medium, or in a mixture system with a water-soluble organic solvent such as methanol, ethanol, isopropyl alcohol, acetonitrile, acetone, and dimethyl sulfoxide. The reaction in the present invention may be performed in hatch, fed-batch, or continuous systems, and it is also possible to use immobilized cells, immobilized enzymes, or membrane reactors.

Purification of 1,3-butanediol produced by the reaction can be performed by appropriately combining the following: separation by centrifugation, filtration etc., organic solvent extraction, chromatographies such as ion exchange chromatography, adsorption using adsorbents, dehydration or agglomeration using dehydrating or agglomerating agents, crystallization, distillation, etc.

For example, after a fermentation liquid containing microorganism cells is subjected to centrifugation or membrane filtration to remove microorganism cells and proteins, 1,3-butanediol can be purified from this aqueous solution by known methods such as concentration and distillation.

All prior art documents cited in the present specification are incorporated herein by reference.

### EXAMPLE

Hereinbelow, the present invention will be specifically described using Examples, but it is not to be construed as being limited thereto.

### Example 1

### Cloning of β-Ketothiolase Gene Derived from Ralstonia Eutropha

Ralstonia eutropha DSM 531 was inoculated in 50 mL of liquid medium consisting of 5 g/L, peptone and 3 g/L meat extract, which had been adjusted to pH 7.0. The cells were cultured with shaking at 30′C. for 21 hours.

The cells were collected from the resulting culture by centrifugation, and genomic DNA was obtained from the cells. The genomic DNA was prepared using Genomic Tip-100/G Kit (QIAGEN).

In order to clone the phbA gene present in the genomic DNA obtained from Ralstonia eutropha DSM 531 (hereinafter referred to as ReTHL gene; DDBJ ID=J04987; amino acid sequence: SEQ ID NO: 21; nucleotide sequence: SEQ ID NO: 22), two PCR primers (ReTHL-A3 and ReTHL-T3) were designed. The nucleotide sequences of the designed sense and antisense primers are shown below:

ReTHL-A3
(SEQ ID NO: 27)
gacggtacctatatATGACTGATGTTGTCATCGTATCC
ReTHL-T3
(SEQ ID NO: 28)
cacaagettaTTATTTACGTTCAACTGCCAGCGC

The ReTHL gene was cloned using the two PCR primers and the genomic DNA of Ralstonia eutropha DSM 531 as a template. Specifically, a 50 μL solution containing the two PCR primers, the genomic DNA, 0.2 mM dNTP, and 2.5 U PfuUltra in PfuUltra reaction buffer was prepared, and subjected to 30 cycles of 95° C. for 30 seconds; 55° C. for 30 seconds; and 72° C. for 1 minute 10 seconds. As a result, a DNA fragment of about 1.2 kb was amplified.

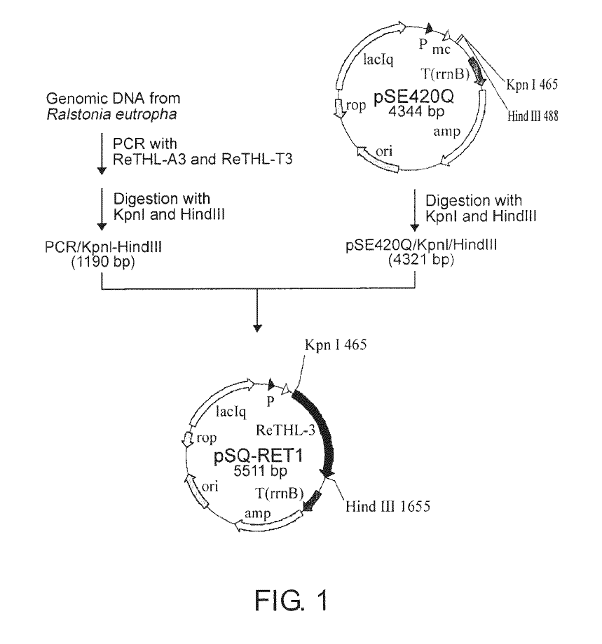

A plasmid containing the whole ReTHL gene was prepared as shown in FIG. 1. Specifically, the DNA fragment obtained by PCR was double-digested with KpnI and HindIII, and ligated with KpnI-HindIII-treated pSE420Q vector (WO 2006-132145) to prepare pSQ-RET1, an expression plasmid for the ReTHL gene.

### Example 2

### Expression of ReTHL gene in E. coli

The plasmid pSQ-RET1 prepared in Example 1 was introduced into E. coli JM109 by the Hanahan method to obtain a transformant E. coli JM109 (pSQ-RET1). This transformant was cultured by the following method:

The transformant was inoculated into a 21 mm diameter test tube containing 7 ml of LB medium consisting of 10 g/L tryptone, 5 g/L yeast extract, and 10 g/L NaCl, which had been adjusted to pH 7.2. The cells were cultured at 30.0° C. for 18 hours while stirring at 250 rpm under aerobic conditions. IPTG was then added at a final concentration of 0.1 mM and incubated at 30.0° C. for 4 hours while stirring at 250 rpm under aerobic conditions to induce the expression of the introduced gene.

The resulting culture was dispensed into a 2 ml Eppendorf tube, and centrifuged to collect the cells. Then, 50 mM potassium phosphate buffer (pH 7.0) containing 1 mM DTT was added to the cells, and subjected to ultrasonication to disrupt the cells. Intact cells and cell debris were removed by centrifugation to obtain a cell-free extract.

The ReTHL activity of the obtained cell-free extract was measured by THL activity assay-1, and determined to be 47.1 U/mg.

### Example 3

### Cloning of β-Ketothiolase Gene Derived from Zoogloea Ramigera

Genomic DNA was obtained from Zoogloea ramigera DSM 287 in the same manner as in Example 1.

In order to clone the phbA gene present in the genomic DNA (hereinafter referred to as ZrTHL gene; DDBJ ID=J02631; amino acid sequence: SEQ ID NO: 23; nucleotide sequence: SEQ ID NO: 24), six PCR primers (ZrTHL-A2, ZrTHL-T2, ZrTHL-Nco-F1, ZrTHL-Nco-F2, ZrTHL-Nco-R1, and ZrTHL-Nco-R2) were designed. The nucleotide sequences of the designed sense and antisense primers are shown below:

ZrTHL-A2
(SEQ ID NO: 29)
gacggtacctatatATGAGTACTCCATCAATCGTC
ZrTHL-T2
(SEQ ID NO: 30)
cacaagcttaTTAAAGACTTTCGATGCACATCGC
ZrTHL-Nco-F1
(SEQ ID NO: 31)
GGAATCCATGTCAATCTGCCCCG
ZrTHL-Nco-F2
(SEQ ID NO: 32)
GCTCGATITCAATGGCGAAGC
ZrTHL-Nco-R1
(SEQ ID NO: 33)
CAATGCGGGGCCATTGACATGG
ZrTHL-Neo-R2
(SEQ ID NO: 34)
CGGAGCTTCGCCATTGAATCGAG

DNA fragments were amplified using the genomic DNA of Zoogloea ramigera DSM 287 as a template. Specifically, a 50 μL solution containing two PCR primers (ZrTHL-A2 and ZrTHL-Nco-R1), the genomic DNA, 0.2 mM dNTP, and 2.5 U PfuUltra in PfuUltra reaction buffer was prepared, and subjected to 30 cycles of 95° C. for 30 seconds; 55° C. for 30 seconds; and 72° C. for 30 seconds. As a result, DNA fragment 1 of about 400 bp was amplified. Another PCR was carried out using two primers (ZrTHL-Nco-F1 and ZrTHL-Nco-R2) in the same manner to amplify DNA fragment 2 of about 300 bp. Another PCR was carried out using two primers (ZrTHL-Nco-F2 and ZrTHL-T2) in the same manner to amplify DNA fragment 3 of about 500 bp.

These three DNA fragments were used as templates to construct the entire ORF of the ZrTHL gene. Specifically, a 50 μL solution, containing the three DNA fragments, two PCR primers (ZrTHL-A2 and ZrTHL-T2), 0.2 mM dNTP, and 3.0 U PfuUltra in PfuUltra reaction buffer was prepared, and subjected to 30 cycles of 95° C. for 30 seconds; 55° C. for 30 seconds; and 72° C. for 1 minute 20 seconds. As a result, a DNA fragment of about 1.2 kb was amplified.

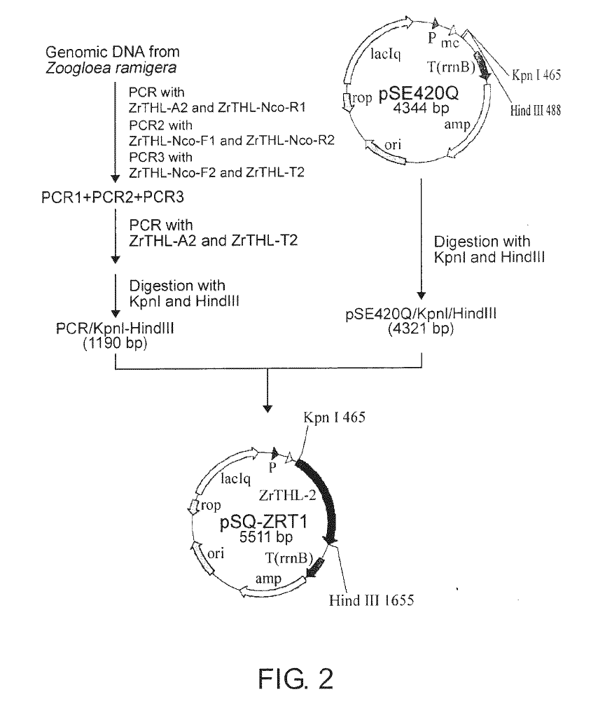

A plasmid containing the ZrTHL, gene was prepared as shown in FIG. 2. Specifically, the DNA fragment obtained by PCR was double-digested with KpnI and HindIII, and ligated with KpnI-HindIII-treated pSE420Q vector (WO 2006-132145) to prepare pSQ-ZRT1, an expression plasmid for the ZrTHL gene.

### Example 4

### Expression of ZrTHL Gene in E. Coli

The plasmid pSQ-ZRT1 prepared in Example 3 was introduced into E. coli JM109 by the Hanahan method to obtain a transformant E. coli JM109 (pSQ-ZRT1). This transformant was cultured by the method described in Example 2.

The culture obtained by the above-described method was dispensed into a 2 ml Eppendorf tube, and centrifuged to collect the cells. Then, 50 mM potassium phosphate buffer (pH 7.0) containing 1 mM DTT was added to the cells, and subjected to ultrasonication to disrupt the cells. Intact cells and cell debris were removed by centrifugation to obtain a cell-free extract.

The ZrTHL activity of the obtained cell-free extract was measured by THL activity assay-1, and determined to be 31.8 U/mg.

### Example 5

### Cloning of β-Ketothiolase Derived from Escherichia Coli

Genomic DNA was obtained from Escherichia coli by the method described in Example 1.

In order to clone the atoB gene present in the obtained genomic DNA (hereinafter referred to as EcTHL gene; DDBJ ID=AP009048; amino acid sequence: SEQ ID NO: 25; nucleotide sequence: SEQ ID NO: 26), two PCR primers (EcTHL-A1 and EcTHL-T1) were designed. The nucleotide sequences of the designed sense and antisense primers are shown below:

EcTHL-A1
(SEQ ID NO: 35)
gacggtacctatatATGAAAAATTGTGTCATCGTCAG
EcTHL-T1
(SEQ ID NO: 36)
cacaagcttaTTAATTCAAGCGTTCAATCACCATC

The EcTHL gene was cloned using the two PCR primers and the genomic DNA of E. coli JM109 as a template. Specifically, a 50 μL solution containing the two PCR primers (EcTHL-A1 and EcTHL-T1), the genomic DNA, 0.2 mM dNTP, and 2.5 U PfuUltra in PfuUltra reaction buffer was prepared, and subjected to 30 cycles of 95° C. for 30 seconds; 50° C. for 30 seconds; and 72° C. for 1 minute 20 seconds. As a result, a DNA fragment of about 1.2 kb was amplified.

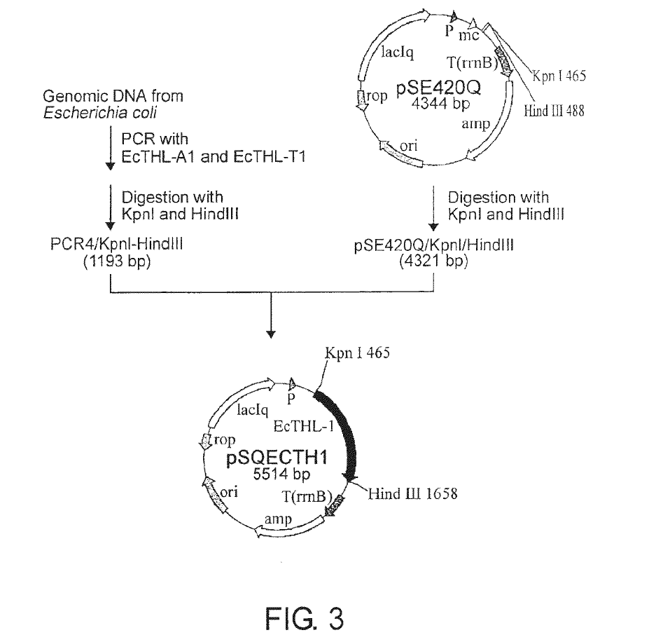

A plasmid containing the EcTHL gene was prepared as shown in FIG. 3. Specifically, the DNA fragment obtained by PCR was double-digested with KpnI and Hindu, and ligated with KpnI-HindIII-treated pSE420Q vector (WO 2006-132145) to prepare pSQECTH1, an expression plasmid for the EcTHL gene.

### Example 6

### Expression of EcTHL Gene in E. coli

The plasmid pSQECTH1 prepared in Example 5 was introduced into E. coli JM109 by the Hanahan method to obtain a transformant E. coli JM109 (pSQECTH1). This transformant was cultured by the method described in Example 2.

The culture obtained by the above-described method was dispensed into a 2 ml. Eppendorf tube, and centrifuged to collect the cells. Then, 50 mM potassium phosphate buffer (pH 7.0) containing 1 mM DTT was added to the cells, and subjected to ultrasonication to disrupt the cells. Intact cells and cell debris were removed by centrifugation to obtain a cell-free extract.

The activity of the obtained cell-free extract was measured by THL activity assay-2, and determined to be 5.61 U/mg

### Example 7

### Cloning of phbB Gene Derived from Ralstonia Eutropha

In order to clone the phbB gene present in the genomic DNA of Ralstonia eutropha DSM 531 prepared in Example 1 thereinafter referred to as ReAR1 gene; DDBJ ID=J04987; amino acid sequence: SEQ ID NO: 9; nucleotide sequence: SEQ ID NO: 10), four PCR primers (ReAR1-A3, ReAR-T3, ReAR-Nco-F1, and ReAR-Nco-R1) were designed. The nucleotide sequences of the designed sense and antisense primers are shown below:

ReAR-A3
(SEQ ID NO: 37)
gaggaattcatatATGACTCAACGTATTGCGTATGTG
ReAR-T3
(SEQ ID NO: 38)
cagactagtaTTAGCCCATGTGCAGGCCG
ReAR-Nco-F1
(SEQ ID NO: 39)
CATGGCTTCACTATGGCACTGGC
RAR-Nco-R1
(SEQ ID NO: 40)
GCCAGTGCCATAGTGAAGCCATG

DNA fragments were amplified using the genomic DNA of Ralstonia eutropha DSM 531 as a template. Specifically, a 50 μL solution containing two PCR primers (ReAR-A3 and ReAR-Nco-R1), the genomic DNA, 0.2 mM dNTP, and 2.5 U PfuUltra in PfuUltra reaction buffer was prepared, and subjected to 30 cycles of 95° C. for 30 seconds; 55° C. for 30 seconds; and 72° C. for 40 seconds. As a result, DNA fragment 1 of about 500 bp was amplified. Another PCR was carried out using two primers (ReAR-Nco-F1 and ReAR-T3) to amplify DNA fragment 2 of about 300 bp.

These two DNA fragments were used as templates to construct the entire ORF of the ZrTHL gene. Specifically, a 50 μL solution containing the two DNA fragments, two PCR primers (ReAR-A3 and ReAR-T3), 0.2 mM dNTP, and 3.0 U PfuUltra in PfuUltra reaction buffer was prepared, and subjected to 30 cycles of 95° C. for 30 seconds; 55° C. for 30 seconds; and 72° C. for 1 minute. As a result, a DNA fragment of about 800 bp was amplified.

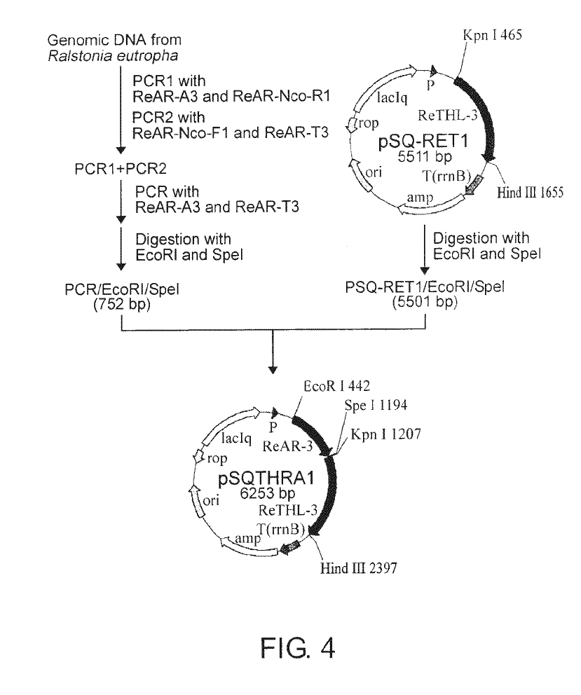

A plasmid containing the ReAR1 gene was prepared as shown in FIG. 4. Specifically, the DNA fragment obtained by PCR was double-digested with EcoRI and SpeI, and ligated with the pSQ-RET1 vector prepared in Example 1 after treating it with EcoRI and SpeI, to prepare pSQTHRA1, a coexpression plasmid carrying the ReTHL gene and ReAR1 gene.

### Example 8

### Expression of ReTHL Gene and ReAR1 Gene in E. coli

The plasmid pSQTHRA1 prepared in Example 7 was introduced into E. coli JM109 by the Hanahan method to obtain a transformant E. coli JM109 (pSQTHRA1). This transformant was cultured by the method described in Example 2.

The obtained culture was dispensed into a 2 ml Eppendorf tube, and centrifuged to collect the cells. Then, 50 mM potassium phosphate buffer (pH 8.0) containing 1 mM DTT was added to the cells, and subjected to ultrasonication to disrupt the cells. Intact cells and cell debris were removed by centrifugation to obtain a cell-free extract.

The obtained cell-free extract was assayed for ReTHL activity by THL activity assay-1 and for ReAR1 activity by the 3HBD activity assay, and the results were 41.3 U/mg and 6.12 U/mg, respectively.

### Example 9

### Cloning of phbB Gene Derived from Zoogloea Ramigera

In order to clone the phbB gene present in the genomic DNA of Zoogloea ramigera DSM 287 prepared in Example 3 (hereinafter referred to as ZrAR1 gene; WO 9100917; amino acid sequence: SEQ ID NO: 11; nucleotide sequence: SEQ ID NO: 12), two PCR primers (ZrAR-A2 and ZrAR-T2) were designed. The nucleotide sequences of the designed sense and antisense primers are shown below:

ZrAR-A2
(SEQ ID NO: 41)
gaggaattcatatATGAGTCGTGTAGCATTGGTAAC
ZrAR-T2
(SEQ ID NO: 42)
cagactagtaTTAGACCEAAGAACTGGCCG

The ZrAR1 gene was cloned using the two PCR primers and the genomic DNA of Zoogloea ramigera as a template. Specifically, a 50 μL solution containing the two PCR primers (ZrAR-A2 and ZrAR-T2), the genomic DNA, 0.2 mM dNTP, and 2.5 U PfuUltra in PfuUltra reaction buffer was prepared, and subjected to 30 cycles of 95° C. for 30 seconds; 53° C. for 30 seconds; and 72° C. for 40 seconds. As a result, a DNA fragment of about 700 bp was amplified.

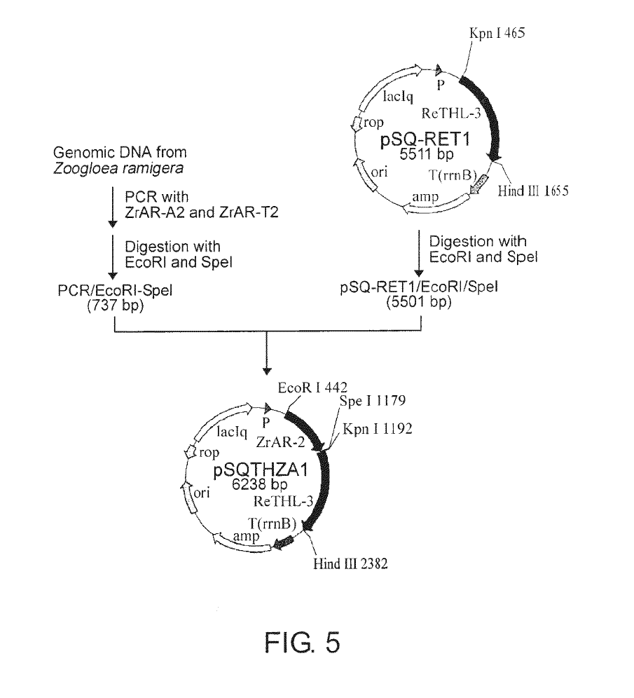

A plasmid containing the ZrAR1 gene was prepared as shown in FIG. 5. Specifically, the DNA fragment obtained by PCR was double-digested with EcoRI and SpeI, and ligated with the pSQ-RET1 vector prepared in Example 1 after treating it with EcoRI and SpeI, to prepare pSQTHZA1, a coexpression plasmid carrying the ReTHL gene and ZrAR1 gene.

### Example 10

### Expression of ReTHL Gene and ZrAR1 Gene in E. Coli

The plasmid pSQTHZA1 prepared in Example 9 was introduced into E. coli JM109 by the Hanahan method to obtain a transformant E. coli JM109 (pSQTHZA1). This transformant was cultured by the method described in Example 2.

The obtained culture was dispensed into a 2 ml Eppendorf tube, and centrifuged to collect the cells. Then, 50 mM potassium phosphate buffer (pH 8.0) containing 1 mM DTT was added to the cells, and subjected to ultrasonication to disrupt the cells. Intact cells and cell debris were removed by centrifugation to obtain a cell-free extract.

The obtained cell-free extract was assayed for ReTHL activity by THL, activity assay-1 and for ZrAR1 activity by the 3HBD activity assay, and the results were 72.9 U/mg and 0.582 U/mg, respectively.

### Example 11

### Cloning of actIII Gene Derived from Streptomyces Violaceoruber

Streptomyces violaceoruber IFO 15146 was inoculated in 50 mL of liquid medium consisting of 4 g/L glucose, 4 g/L yeast extract, and 10 malt extract (YM medium), which had been adjusted to pH 7.2. The cells were cultured with shaking at 28° C. for 24 hours.

The cells were collected from the resulting culture by centrifugation, and genomic DNA was obtained from the cells. The genomic DNA was prepared using Genomic Tip-100/G Kit (QIAGEN).

In order to clone the actin gene present in the obtained genomic DNA (hereinafter referred to as SvKR1 gene; DDBJ ID=M19536; amino acid sequence: SEQ ID NO: 15; nucleotide sequence: SEQ ID NO: 16), two PCR primers (SvKR-A4 and SvKR-T4) were designed. The nucleotide sequences of the designed sense and antisense primers are shown below:

SvKR-A4
(SEQ ID NO: 43)
gaggaattcatatATGGCCACGCAGGACTCC
SyKR-T4
(SEQ ID NO: 44)
cagactagtaTTAGTAGTTCCCCAGCCCG

The SvKR1 gene was cloned using the two PCR primers and the genomic DNA of Streptomyces violaceoruber as a template. Specifically, a 50 μL solution containing the two PCR primers (SvKR-A4 and SvKR-T4), the genomic DNA, 0.2 mM dNTP, and 2.5 U PfuUltra in PfuUltra reaction buffer was prepared, and subjected to 30 cycles of 95° C. for 30 seconds; 55° C. for 30 seconds; and 72° C. for 1 minute. As a result, a DNA fragment of about 800 bp was amplified.

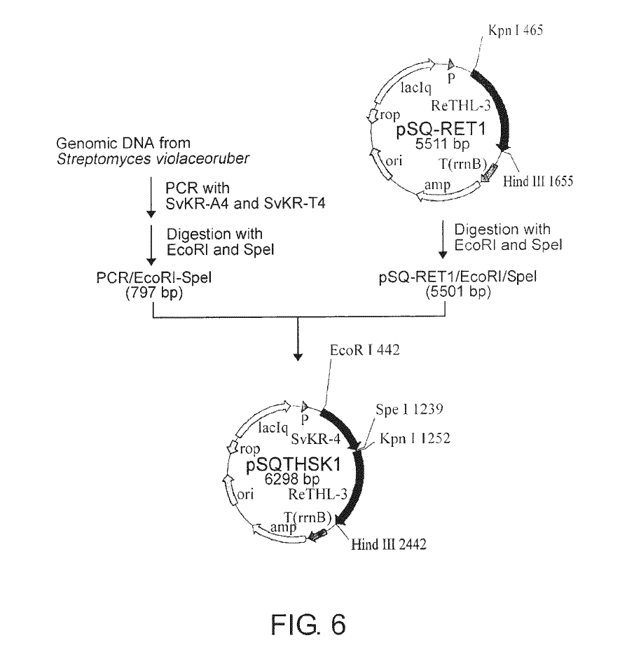

A plasmid containing the SvKR1 gene was prepared as shown in FIG. 6. Specifically, the DNA fragment obtained by PCR was double-digested with EcoRI and SpeI, and ligated with the pSQ-RET1 vector prepared in Example 1 after treating it with EcoRI and SpeI, to prepare pSQTHSK1, a coexpression plasmid carrying the ReTHL gene and the SvKR1 gene.

### Example 12

### Expression of ReTHL Gene and SvKR1 Gene in E. coli

The plasmid pSQTHSK1 prepared in Example 11 was introduced into E. coli JM109 by the Hanahan method to obtain a transformant E. coli JM109 (pSQTHSK1). This transformant was cultured by the method described in Example 2.

The obtained culture was dispensed into a 2 ml Eppendorf tube, and centrifuged to collect the cells. Then, 50 mM potassium phosphate buffer (pH 8.0) containing 1 mM DTT was added to the cells, and subjected to ultrasonication to disrupt the cells. Intact cells and cell debris were removed by centrifugation to obtain a cell-free extract.

The obtained cell-free extract was assayed for ReTHL activity by THL activity assay-1 and for SvKR1 activity by the 3HBD activity assay, and the results were 7.71 U/mg and 0.0547 U/mg, respectively.

### Example 13

### Cloning of β-Ketoacyl-ACP Reductase Gene Derived from Geobacillus Stearothermophilus

Geobacillus stearothermophilus NBRC 12550 was inoculated in 50 mL of liquid medium consisting of 10 g/L polypeptone, 2 g/L yeast extract, and 1 g/L MgSO₄.H₂O, which had been adjusted to pH 7.0. The cells were cultured with shaking at 50° C. for 21 hours.

The cells were collected from the resulting culture by centrifugation, and genomic DNA was obtained from the cells. The genomic DNA was prepared using Genomic Tip-100/G Kit (QIAGEN).

In order to clone the β-ketoacyl-ACP reductase gene present in the obtained genomic DNA (hereinafter referred to as BstKR1 gene; JP-A (Kokai) 2002-209592; amino acid sequence: SEQ ID NO: 13; nucleotide sequence: SEQ ID NO: 14), two PCR primers (BstKR-A3 and BstKR-T3) were designed. The nucleotide sequences of the designed sense and antisense primers are shown below:

BstKR-A3
(SEQ ID NO: 45)
gaggaattcatatATGTCTCAACGTTTTGCAGGTC
BstKR-T3
(SEQ ID NO: 46)
cagactcgtaTTAACATTITGGACCACCTGC

The BstKR1 gene was cloned using the two PCR primers and the genomic DNA of Geobacillus stearothermophilus as a template. Specifically, a 50 μL solution containing the two PCR primers (BstKR-A3 and BstKR-T3), the genomic DNA, 0.2 mM dNTP, and 2.5 U PfuUltra in PfuUltra reaction buffer was prepared, and subjected to 30 cycles of 95° C. for 30 seconds; 50° C. for 30 seconds; and 72° C. for 1 minute. As a result, a DNA fragment of about 800 bp was amplified.

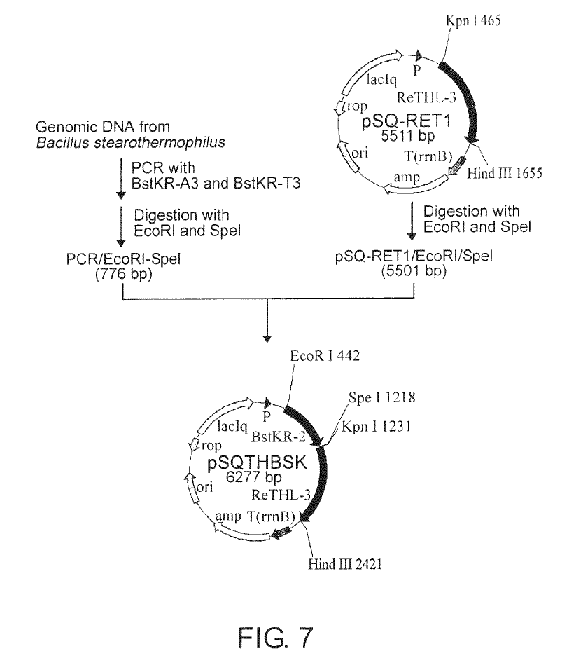

A plasmid containing the BstKR1 gene was prepared as shown in FIG. 7. Specifically, the DNA fragment obtained by PCR was double-digested with EcoRI and SpeI, and ligated with the pSQ-RET1 vector prepared in Example 1 after treating it with EcoRI and SpeI, to prepare pSQTHBSK, a coexpression plasmid carrying the ReTHL gene and the BstKR1 gene.

### Example 14

### Expression of ReTHL Gene and BstKR1 Gene in E. coli

The plasmid pSQTHBSK prepared in Example 13 was introduced into E. coli JM109 by the Hanahan method to obtain a transformant E. coli JM109 (pSQTHBSK). This transformant was cultured by the method described in Example 2.

The culture obtained by the above-mentioned method was dispensed into a 2 ml Eppendorf tube, and centrifuged to collect the cells. Then, 50 mM potassium phosphate buffer (pH 8.0) containing 1 mM DTT was added to the cells, and subjected to ultrasonication to disrupt the cells. Intact cells and cell debris were removed by centrifugation to obtain a cell-free extract.

The obtained cell-free extract was assayed for ReTHL activity by THL activity assay-1 and for BstKR1 activity by the 3HBD activity assay, and the results were 99.0 U/mg and 1.494 U/mg, respectively.

### Example 15

### Cloning of 3-Hydroxybutyryl-CoA Dehydrogenase Gene Derived from Clostridium Acetobutylicum

In order to clone the 3-hydroxybutyryl-CoA dehydrogenase gene present in the genomic DNA purchased from ATCC (hereinafter referred to as CaHBD gene; DDBJ ID=AE001437; amino acid sequence: SEQ ID NO: 13; nucleotide sequence: SEQ ID NO: 14), two PCR primers (CaHBD-A1 and CaHBD-T1) were designed. The nucleotide sequences of the designed sense and antisense primers are shown below:

CaHBD-A1
(SEQ ID NO: 47)
gaggaattcatatATGAAAAAGGTATGTGTTATAGGTGC
CaHBD-T1
(SEQ ID NO: 48)
cagactagtaTTATTTTGAATAATCGTAGAAACCTTTTCC

The CaHBD gene was cloned using the two PCR primers and the genomic DNA of Clostridium acetobutylicum as a template. Specifically, a 50 μL solution containing the two PCR primers (CaHBD-A1 and CaHBD-T1), the genomic DNA, 0.2 mM dNTP, and 2.5 U PfuUltra in PfuUltra reaction buffer was prepared, and subjected to 30 cycles of 95° C. for 30 seconds; 50° C. for 30 seconds; and 72° C. for 1 minute. As a result, a DNA fragment of about 900 bp was amplified.

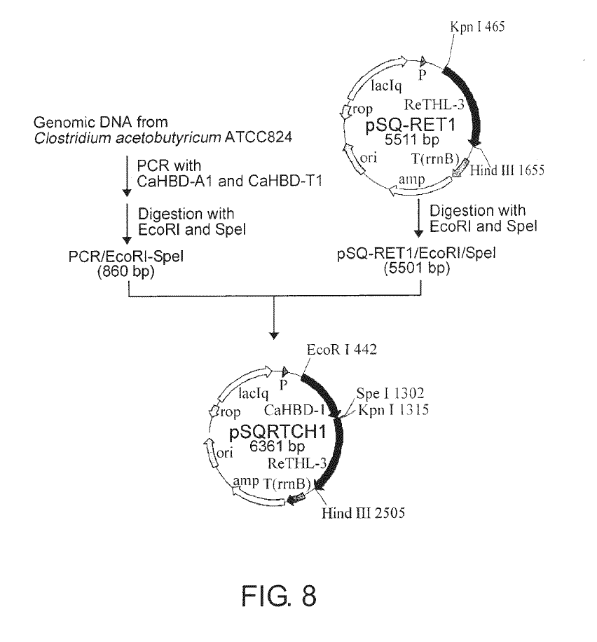

A plasmid containing the CaHBD gene was prepared as shown in FIG. 8. Specifically, the DNA fragment obtained by PCR was double-digested with EcoRI and SpeI, and ligated with the pSQ-RET1 vector prepared in Example 1 after treating it with EcoRI and SpeI, to prepare pSQRTCH1, a coexpression plasmid carrying the ReTHL gene and the CaHBD gene.

### Example 16

### Expression of ReTHL Gene and CaHBD Gene in E. coli

The plasmid pSQRTCH1 prepared in Example 15 was introduced into E. coli JM109 by the Hanahan method to obtain a transformant E. coli JM109 (pSQRTCH1). This transformant was cultured by the method described in Example 2.

The culture obtained by the above-mentioned method was dispensed into a 2 ml Eppendorf tube, and centrifuged to collect the cells. Then, 50 mM potassium phosphate buffer (pH 8.0) containing 1 mM DTT was added to the cells, and subjected to ultrasonication to disrupt the cells. Intact cells and cell debris were removed by centrifugation to obtain a cell-free extract.

The obtained cell-free extract was assayed for ReTHL activity by THL activity assay-1 and for ReAR1 activity by the 3HBD activity assay, and the results were 13.8 U/mg and 63.2 U/mg, respectively.

### Example 17

### Cloning of (R)-2-Octanol Dehydrogenase Gene Derived from Pichia Finlandica

Pichia finlandica DSM 70280 was inoculated in 50 mL of liquid medium consisting of 10 g/L, glucose, 5 g/L peptone (derived from soybean), 3 g/L yeast extract, and 3 g/L malt extract, which had been adjusted to pH 7.0. The cells were cultured with shaking at 25° C. for 24 hours.

The cells were collected from the resulting culture by centrifugation, and genomic DNA was obtained from the cells. The genomic DNA was prepared using Genomic Tip-100/G Kit (QIAGEN).

In order to clone the (R)-2-octanol dehydrogenase gene present in the obtained germane DNA (hereinafter referred to as PfODH gene; DDBJ ID=AB259114; amino acid sequence: SEQ ID NO: 17; nucleotide sequence: SEQ ID NO: 18), six PCR primers (PfODH-A3, PfODH-T3, PfODH-Xba-F1, PfODH-Xba-R1, PfODH-Hind-F1, and PfOHD-Hind-R1) were designed. The nucleotide sequences of the designed sense and antisense primers are shown below:

PfODH-A3
(SEQ ID NO: 49)
gaggAATTCTAAAATGTCTTATAATTTCCATAACAAGGTTGC
PfODH-T3
(SEQ ID NO: 50)
tcgACTAGTATTATTGTGCTGTGTACCCACCGTCAACC
PfODH-Xba-F1
(SEQ ID NO: 51)
GGCTCTGGAGTACGCATCTCATGGTATTCGTGTAAATTC
PfODH-Xba-R1
(SEQ ID NO: 52)
GAATTTACACGAATACCATGAGATGCGTACTCCAGAGCC
PfODH-Hind-F1
(SEQ ID NO: 53)
GTAAGCCTGCACCCTATTGGGCGTCIGGGTCGTC
PfODH-Hind-R1
(SEQ ID NO: 54)
GACGACCCAGACGCCCAATAGGOTGCAGGCTTAC

DNA fragments were amplified using the genomic DNA of Pichia finlandica DSM 70280 as a template. Specifically, a 50 μL solution containing two PCR primers (PfODH-A3 and PfODH-Xba-R1), the genomic DNA, 0.2 mM dNTP, and 2.5 U PfuUltra in PfuUltra reaction buffer was prepared, and subjected to 30 cycles of 95° C. for 30 seconds; 55° C. for 30 seconds; and 72° C. for 30 seconds. As a result. DNA fragment 1 of about 500 bp was amplified. Another PCR was performed using two PCR primers (PfODH-Xba-F1 and PfODH-Hind-R1) to amplify DNA fragment 2. Another PCR was performed using two PCR primers (PfODH-Hind-F1 and PfODH-T3) to amplify DNA fragment 3 of about 200 bp.

These three DNA fragments were used to construct the entire ORF of the PfODH gene. Specifically, a 50 μL solution containing the three DNA fragments, two PCR primers (PfODH-A3 and PfODH-T3), 0.2 mM dNTP, and 3.0 U PfuUltra in PfuUltra reaction buffer was prepared, and subjected to 30 cycles of 95° C. for 30 seconds; 55° C. for 30 seconds; and 72° C. for 1 minute. As a result, a DNA fragment of about 800 bp was amplified.

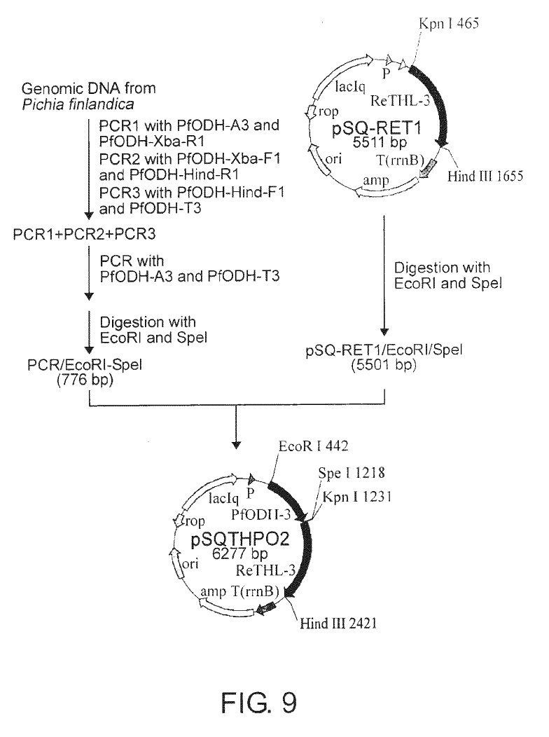

A plasmid containing the PfODH gene was prepared as shown in FIG. 9. Specifically, the DNA fragment obtained by PCR was double-digested with EcoRI and SpeI, and ligated with the pSQ-RET1 vector prepared in Example 1 after treating it with EcoRI and SpeI, to prepare pSQTHPO2, a coexpression plasmid carrying the ReTHL gene and the PfODH gene.

### Example 18

### Expression of ReTHL Gene and PfODH Gene in E. Coli

The plasmid pSQTHPO2 prepared in Example 17 was introduced into E. coli JM109 by the Hanahan method to obtain a transformant E. coli JM109 (pSQTHPO2). This transformant was cultured by the method described in Example 2.

The culture obtained by the above-mentioned method was dispensed into a 2 ml Eppendorf tube, and centrifuged to collect the cells. Then, 50 mM potassium phosphate buffer (pH 8.0) containing 1 mM DTT was added to the cells, and subjected to ultrasonication to disrupt the cells. Intact cells and cell debris were removed by centrifugation to obtain a cell-free extract.

The obtained cell-free extract was assayed for ReTHL activity by THL activity assay-1 and for PfODH activity by the 3HBD activity assay, and the results were 74.9 U/mg and 0.0120 U/mg, respectively.

### Example 19

### Production of 3-Hydroxybutyryl-CoA in Enzymatic Reaction Using ReTHL-CaHBD

To a solution containing 100 mM Tris-HCl buffer (pH 7.4), 2.5 mM NADH, and 2.5 mM acetyl-CoA, 6.2 μL of the cell-free extract containing ReTHL and CaHBD prepared according to Example 14 was added, and reacted at 30° C. for 1 hour. The reaction was terminated by adding 5 μL of 60% (v/v) perchloric acid and 5 μL of 5N aqueous sodium hydroxide solution to the reaction solution. To this reacted solution, 510 μL of 200 mM potassium phosphate buffer was added, and centrifuged to obtain a supernatant. HPLC analysis of the supernatant showed that 0.27 mM 3-hydroxybutyryl-CoA was produced.

The HPLC conditions were as follows:

HPLC column: Wakosil-II 5C18HG (4.6 mm×150 mm) manufactured by Wako Pure Chemical Industries, Ltd.

Eluent: 50 mM phosphate buffer (pH 5.0): acetonitrile=95:5

Column temperature: 30° C.

Flow rate: 1.0 mL/min

Detection: UV absorption at 254 nm

Under the above conditions, 3-hydroxybutyryl-CoA was eluted at 15.3 min. The accumulated concentration was determined based on a calibration curve obtained using 3-hydroxybutyryl-CoA (Sigma).

### Example 20

### Cloning of Aldehyde/Alcohol Dehydrogenase Gene Derived from Clostridium acetobutylicum

In order to clone the aldehyde/alcohol dehydrogenase gene present in the megaplasmid obtained in Example 15 (hereinafter referred to as CaAdhE gene; DDBJ ID=AE001438; amino acid sequence: SEQ NO: 1; nucleotide sequence: SEQ ID NO: 2), four PCR primers (CaAdhE2-A1, CaAdhE2-T1, CaAdhE2-Nde-F1, and CaAdhE2-Nde-R1) were designed. The nucleotide sequences of the designed sense and antisense primers are shown below:

CaAdhE2-A1
(SEQ ID NO: 55)
gaccatATGAAAGTTACAAATCAAAAAGAACTAAAAC
CaAdhE2-T1
(SEQ ID NO: 56)
ctgttaTTAAAATGATTTTATATAGATATCCTTAAGTTC
CaAdbE2-Nde-F1
(SEQ ID NO: 57)
CTATAGAAGCATACGTTTCGG
CaAdhE2-Nde-R1
(SEQ ID NO: 58)
CCGAAACGTATGCTTCTATAG

DNA fragments were amplified using the megaplasmid DNA of Clostridium acetobutylicum ATCC 824 as a template. Specifically, a 50 μL solution containing two PCR primers (CaAdhE2-A1 and CaAdhE2-Nde-R1), the genomic DNA, 0.2 mM dNTP, and 2.5 U PfuUltra in PfuUltra reaction buffer was prepared, and subjected to 30 cycles of 95° C. for 30 seconds; 50° C. for 30 seconds; and 72° C. for 2 minute. As a result, DNA fragment 1 of about 2 kb was amplified. Another PCR was performed using two PCR primers (CaAdhE2-Nde-F1 and CaAdhE2-T1) to amplify DNA fragment 2 of about 600 bp.

These two DNA fragments were used to construct the entire ORF of the CaAdhE gene. Specifically, a 50 μL solution containing the two DNA fragments, two PCR primers (CaAdhE2-A1 and CaAdhE2-T1), 0.2 mM dNTP, and 3.0 U PfuUltra in PfuUltra reaction buffer was prepared, and subjected to 30 cycles of 95° C. for 30 seconds; 50° C. for 30 seconds; and 72° C. for 2 minute 30 seconds. As a result, a DNA fragment of about 2.6 kb was amplified.

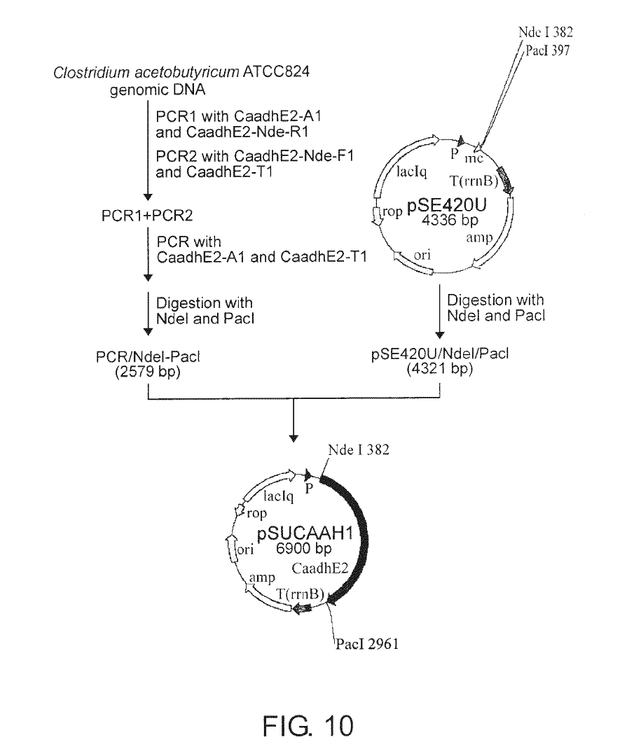

A plasmid containing the CaAdhE gene was prepared as shown in FIG. 10. Specifically, the DNA fragment obtained by PCR was double-digested with NdeI and PacI and ligated with NdeI-PacI-treated pSE420U vector (WO 2006-132145) to prepare pSUCAAH1, an expression plasmid carrying the CaAdhE gene.

### Example 21

### Expression of CaAdhE Gene in E. coli

The plasmid pSUCAAH1 prepared in Example 20 was introduced into E. coli JM109 by the Hanahan method to obtain a transformant E. coli JM109 (pSUCAAH1). This transformant was cultured by the method described in Example 2.

The culture obtained by the above-mentioned method was dispensed into a 2 ml Eppendorf tube, and centrifuged to collect the cells. Under anaerobic conditions, 50 mM MOPS buffer (pH 7.0) containing 1 mM DTT was added to the cells, and subjected to ultrasonication to disrupt the cells. Intact cells and cell debris were removed by centrifugation to obtain a cell-free extract.

The BCDH activity of the obtained cell-free extract was measured by the BCDH activity assay using butyryl-CoA as a substrate, and determined to be 0.129 U/mg.

The CaAdhE gene is known to act on acetyl-CoA and butyryl-CoA. However, its activity on 3-hydroxybutyryl-CoA has never been reported and is therefore unknown. Thus, the BCDH activity was measured using 3-hydroxybutyryl-CoA as a substrate. As a result, the activity was 0.0237 U/mg, which was found to be 18% relative to the activity on butyryl-CoA

### Example 22

### Production of 1,3-BG in Enzymatic Reaction using CaAdhE

To a solution containing 50 mM MES buffer (pH 6.0), 36 mM NADH, and 15 mM 3-hydroxybutyryl-CoA, 0.0938 U of the cell-free extract containing the CaAdhE enzyme prepared according to Example 21 was added, and reacted at 37° C. for 24 hours. The reaction solution was centrifuged to remove precipitate, and the supernatant was analyzed by HPLC. The result showed that 3.66 mM 1,3-BG was produced.

The HPLC conditions were as follows:

HPLC column: ULTRON PS-80H (8.0 mm×300 mm) manufactured by Shinwa Chemical Industries Ltd.

Eluent: 10 mM aqueous sulfuric acid solution

Column temperature: 40° C.

Flow rate: 0.7 mL/min

Detection: RI (refractive index detector)

Under the above conditions, 1,3-BG was eluted at 20.1 min. The accumulated concentration was determined based on a calibration curve obtained using 1,3-BG (Wako).

### Example 23

### Cloning of Aldehyde/Alcohol Dehydrogenase Gene Derived from Thermoanaerobacter pseudethanolicus

In order to clone the aldehyde/alcohol dehydrogenase gene present in the genomic DNA purchased from DSM (hereinafter referred to as TpAdhE gene; amino acid sequence: SEQ ID NO: 65; nucleotide sequence: SEQ ID NO: 66), two PCR primers (TpadhE-A1 and TpAdhE-T1) were designed. The nucleotide sequences of the designed sense and antisense primers are shown below:

TpAdhE-A1
(SEQ ID NO: 69)
gaccatATGCCTAACTTATTACAAGAACGCCGCGAAGTAAAAGA
TpAdhE-T1
(SEQ ID NO: 70)
gctattaattaaTTATTCTCCATAGGCTTTGCGATATATTTCTGC

PCR amplification of the TpAdhE gene was performed using the genomic DNA of Thermoanaerobacter pseudethanolicus as a template. Specifically, a 50 μL solution containing two PCR primers (TpadhE-A1 and TpAdhE-T1), the genomic DNA, 0.2 mM dNTP, and 25 U PfuUltra in PfuUltra reaction buffer was prepared, and subjected to 30 cycles of 95° C. for 30 seconds; 50° C. for 30 seconds; and 72° C. for 2 minutes. As a result, a DNA fragment of about 2.6 kb was amplified.

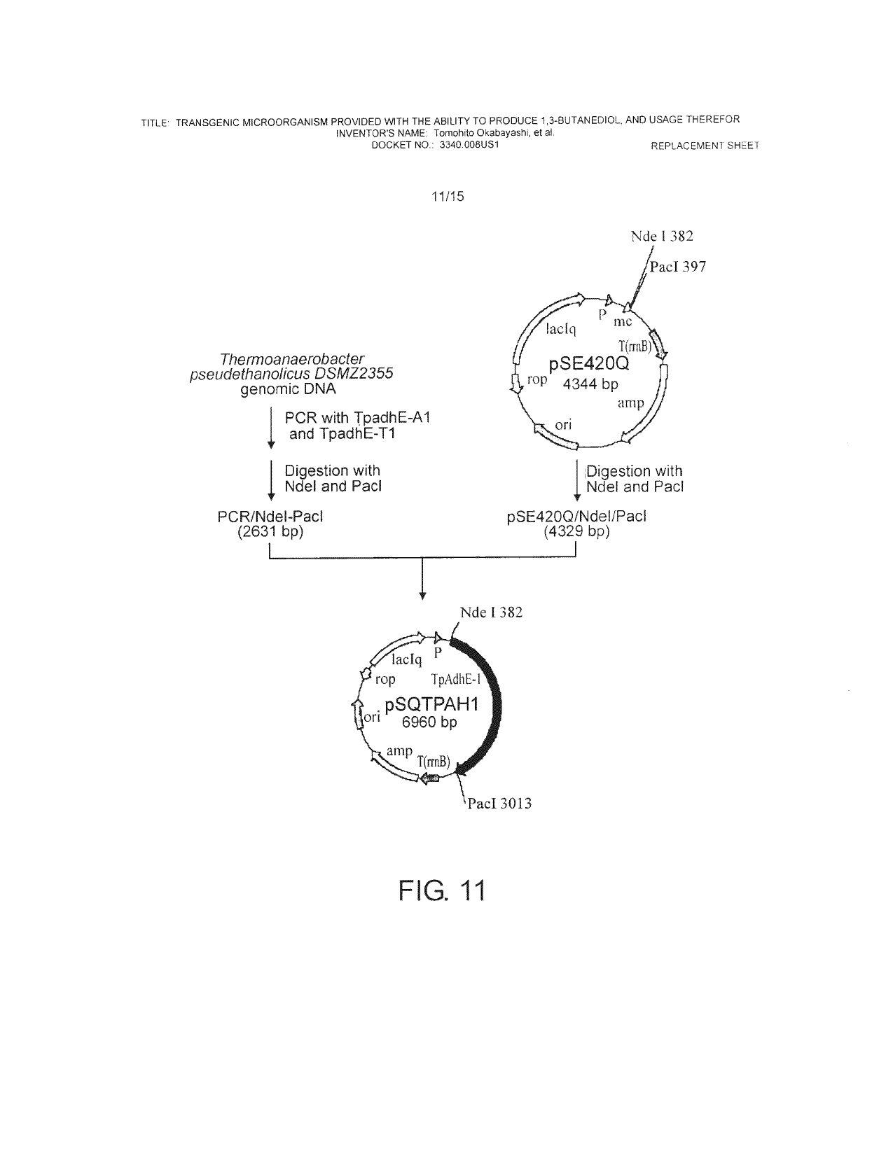

A plasmid containing the TpAdhE gene was prepared as shown in FIG. 11. Specifically, the DNA fragment obtained by PCR was double-digested with NdeI and PacI, and ligated with NdeI-PacI-treated pSE420Q (WO 2006-132145) to prepare pSQTPAH1, an expression plasmid carrying the TpAdhE gene.

### Example 24

### Expression of TpAdhE Gene in E. Coli

The plasmid pSQTPAH1 prepared in Example 23 was introduced into E. coli JM109 by the Hanahan method to obtain a transformant E. coli JM109 (pSQTPAH1). This transformant was cultured by the method described in Example 2.

The culture obtained by the above-mentioned method was dispensed into a 2 ml Eppendorf tube, and centrifuged to collect the cells. Under anaerobic conditions, 50 mM MOPS buffer (pH 7.0) containing 1 mM DTT was added to the cells, and subjected to ultrasonication to disrupt the cells. Intact cells and cell debris were removed by centrifugation to obtain a cell-free extract.

The BCDH/BDH activity of TpAdhE in the obtained cell-free extract on 3-hydroxybutyryl-CoA was determined to be 0.00538 U/mg, and the BDH activity on prepared 3-hydroxybutylaldehyde was 0.0619 U/mg.

### Example 25

### Cloning of Aldehyde/Alcohol Dehydrogenase Gene Derived from Propionibacterium Freudenreichii subsp. Freudenreichii

In order to clone the aldehyde/alcohol dehydrogenase gene present in the genomic DNA purchased from DSM (hereinafter referred to as PfALD gene; amino acid sequence: SEQ ID NO: 67; nucleotide sequence: SEQ ID NO: 68), two PCR primers (PfadhE-A1 and PfAdhE-T1) were designed. The nucleotide sequences of the designed sense and antisense primers are shown below:

PfAdhE-A1
(SEQ ID NO: 71)
gaccatATGGATTTCTCATTGACCGAAGACCAGCAG
PfAdhE-T1
(SEQ ID NO: 72)
gctgttaaTTAACTACCCTTAGTCGCGCAGTGCACC

PCR amplification of the PfALD gene was performed using the genomic DNA of Propionibacterium freudenreichii subsp. freudenreichii as a template. Specifically, a 50 μL solution containing two PCR primers (PfadhE-A1 and PfAdhE-T1), the genomic DNA, 0.2 mM dNTP, and 2.5 U PfuUltra in PfuUltra reaction buffer was prepared, and subjected to 30 cycles of 95° C. for 30 seconds; 50° C. for 30 seconds; and 72° C. for 2 minutes. As a result, a DNA fragment of about 1.1 kb was amplified.

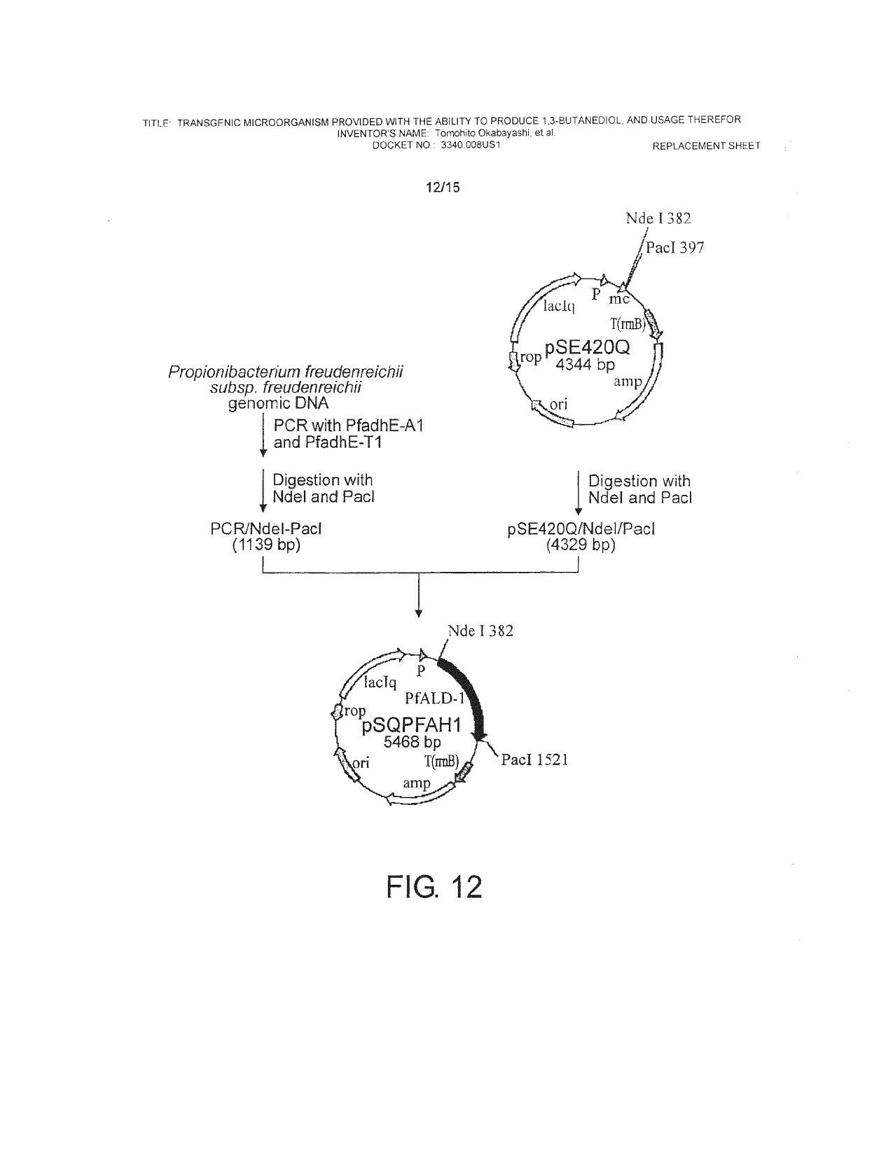

A plasmid containing the PfALD gene was prepared as shown in FIG. 12. Specifically, the DNA fragment obtained by PCR was double-digested with NdeI and PacI, and ligated with NdeI-PacI-treated pSE420Q (WO 2006-432145) to prepare pSQPFAH1, an expression plasmid carrying the PfALD gene.

### Example 26

### Expression of PfALD Gene in E. Coli

The plasmid pSQPFAH1 prepared in Example 25 was introduced into E. coli JM109 by the Hanahan method to obtain a transformant E. coli JM109 (pSQPFAH1). This transformant was cultured by the method described in Example 2.

The culture obtained by the above-mentioned method was dispensed into a 2 ml Eppendorf tube, and centrifuged to collect the cells. Under anaerobic conditions, 50 mM MOPS buffer (pH 7.0) containing 1 mM DTT was added to the cells, and subjected to ultrasonication to disrupt the cells. Intact cells and cell debris were removed by centrifugation to obtain a cell-free extract.

The BCDH activity of PfALD in the obtained cell-free extract on 3-hydroxybutyryl-CoA was determined to be 0.0826 U/mg.

### Example 27

### Plasmid Construction for Fermentative Production of 1,3-BG

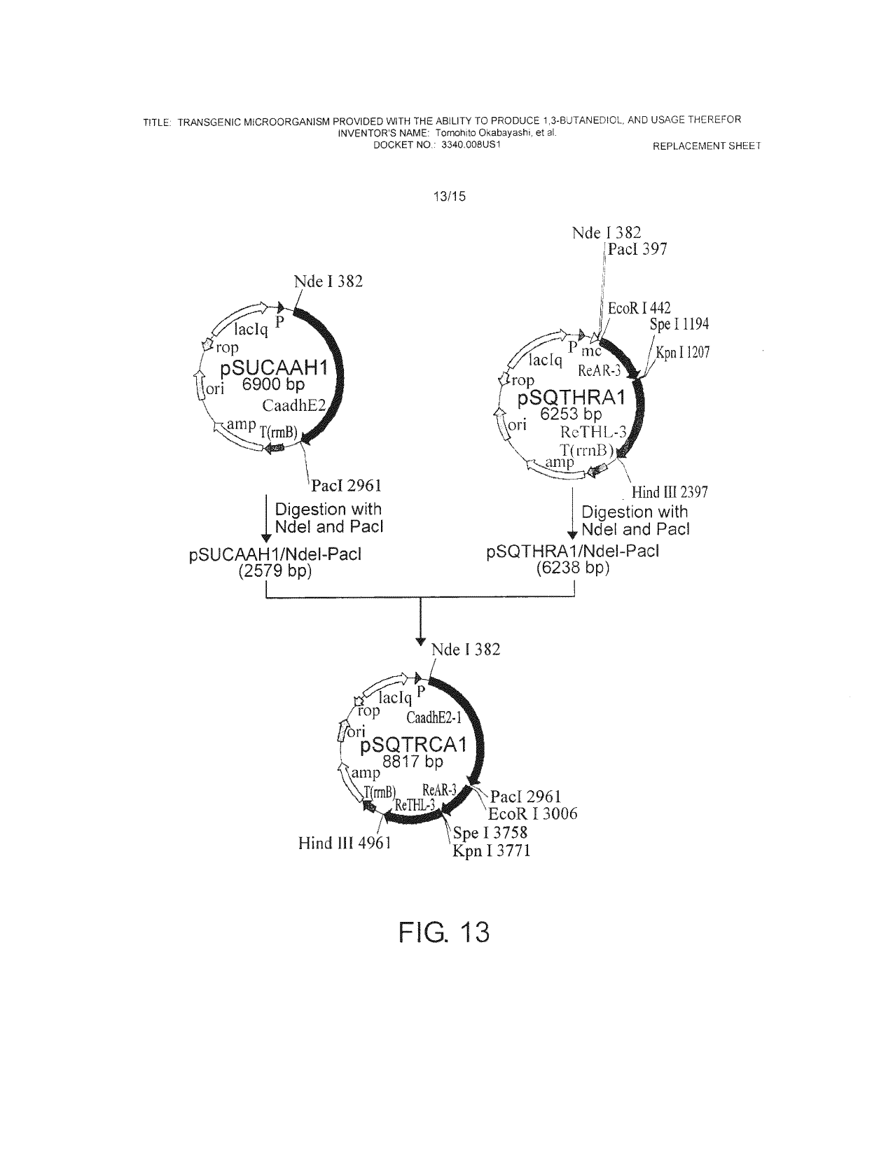

In order to construct a plasmid for fermentative production of 1,3-BG, the CaAdhE gene in the pSUCAAH1 plasmid produced in Example 20 was subcloned into pSQTHRA1 by the method shown in FIG. 13. Specifically, pSUCAAH1 was double-digested with NdeI and PacI, and ligated with NdeI-PacI-treated pSQTHRA1 vector to construct pSQTRCA1, a coexpression plasmid carrying the ReTHL gene, ReAR1 gene, and CaAdhE gene.

### Example 28

### Expression of ReTHL Gene, ReAR1 Gene, and CaAdhE Gene in E. Coli

The plasmid pSQTHRA1 prepared in Example 27 was introduced into E. coli JM109 by the Hanahan method to obtain a transformant E. coli JM109 (pSQTHRA1). This transformant was cultured by the method described in Example 2.

The culture obtained by the above-mentioned method was dispensed into a 2 ml Eppendorf tube, and centrifuged to collect the cells. Under anaerobic conditions, 50 mM MOPS buffer (pH 7.0) containing 1 mM DTT was added to the cells, and subjected to ultrasonication to disrupt the cells. Intact cells and cell debris were removed by centrifugation to obtain a cell-free extract.

The obtained cell-free extract was assayed for ReTHL activity by THL activity assay-1, for ReAR1 activity by the 3HBD activity assay, and for BCDH activity of CaAdhE by the BCDH activity assay using butyryl-CoA as a substrate, and the results were 6.46 U/mg, 0.952 U/mg, and 0.118 U/mg, respectively.

### Example 29

### Cloning of Butanol Dehydrogenase II Gene Derived from Clostridium Acetobutylicum

In order to clone the bdhB gene from C. acetobutylicum (hereinafter referred to as CaBDHB gene; DDBJ ID=AE001437; amino acid sequence: SEQ ID NO: 3; nucleotide sequence: SEQ ID NO: 4), six PCR primers (CaBDHB-A2. CaBDHB-T2, CaBDHB-Nco-F1, CaBDHB-Nco-R1, CaBDHB-Xba-F1, and CaBDHB-Xba-R1) were designed. The nucleotide sequences of the designed sense and antisense primers are shown below:

CaBDHB-A2
(SEQ ID NO: 59)
ggaccATGGTTGATTTCGAATATTCAATACCAACTAGAA
CaBDHB-T2
(SEQ ID NO: 60)
cgatctagaaTTACACAGATTTTTTGAATATTTGTAGGACTTCGGAG
CaBDHB-Nco-F1
(SEQ ID NO: 61)
GATGGAAATCCGTGGGATATTGTG
CaBDHB-Nco-R1
(SEQ ID NO: 62)
CACAATATCCCACGGATTTCCATC
CaBDHB-Xba-F1
(SEQ ID NO: 63)
GTTTACCATCTCGTCTGCGTGATGTTG
CaBDHB-Xba-R1
(SEQ ID NO: 64)
CAACATCACGCAGACGAGATGGTAAAC

DNA fragments were amplified using the genomic DNA of Clostridium acetobutylicum as a template. Specifically, a 50 μL solution containing two PCR primers (CaBDHB-A2 and CaBDHB-Nco-R1), the genomic DNA, 0.2 mM dNTP, and 2.5 U PfuUltra in PfuUltra reaction buffer was prepared, and subjected to 30 cycles of 95° C. for 30 seconds; 50° C. for 30 seconds; and 72° C. for 30 seconds. As a result, DNA fragment 1 of about 350 bp was amplified. Another PCR was performed using two PCR primers (CaBDHB-Nco-F1 and CaBDHB-Xba-R1) to amplify DNA fragment 2. Another PCR was performed using two PCR primers (CaBDHB-Xba-F1 and CaBDHB-T2) to amplify DNA fragment 3 of about 100 bp.

These three DNA fragments were used to construct the entire ORF of the CaBDHB gene. Specifically, a 50 μL solution containing the two DNA fragments, two PCR primers (CaBDHB-A2 and CaBDHB-T2), 0.2 mM dNTP, and 3.0 U PfuUltra in PfuUltra reaction buffer was prepared, and subjected to 30 cycles of 95° C. for 30 seconds; 50° C. for 30 seconds; and 72° C. for 1 minute 30 seconds. As a result, a DNA fragment of about 1.2 kb was amplified.

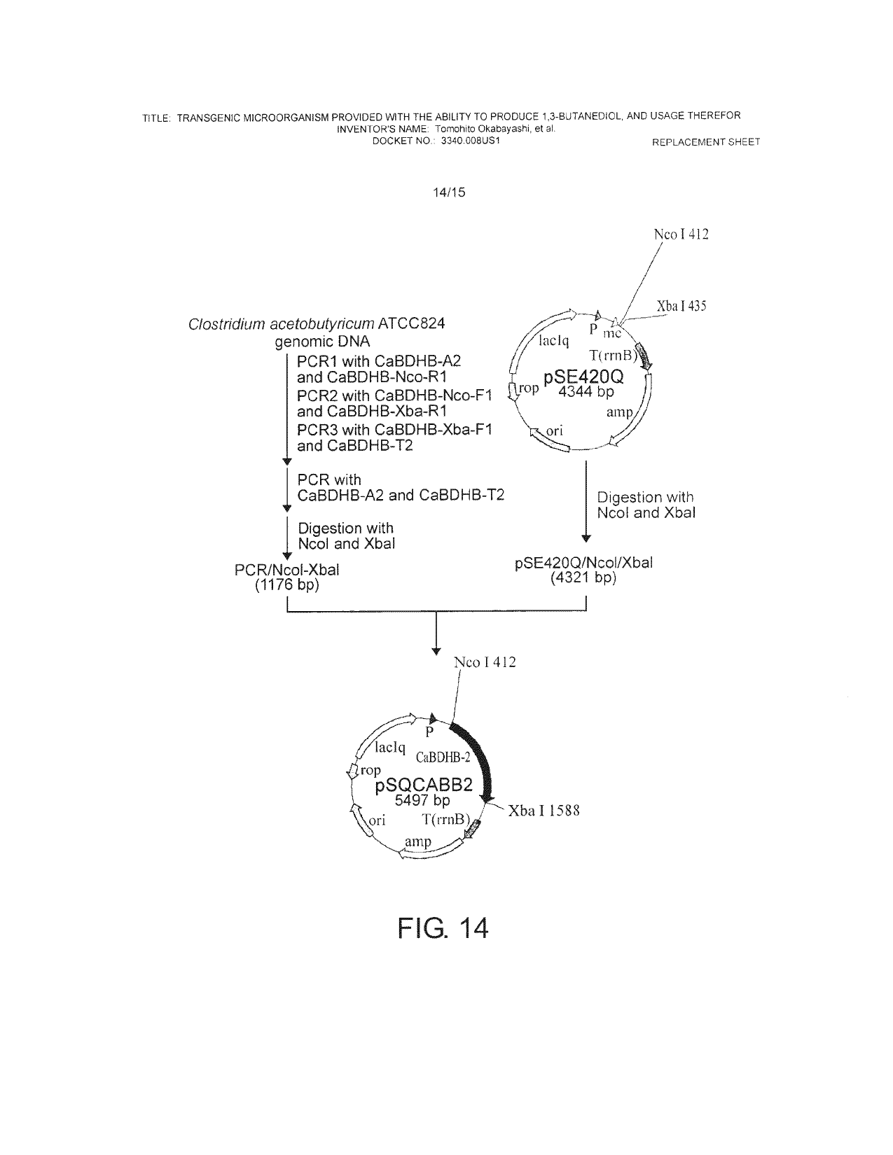

A plasmid containing the whole CaBDHB gene was prepared as shown in FIG. 14. Specifically, the DNA fragment obtained by PCR was double-digested with NcoI and XbaI, and ligated with NcoI-XbaI-treated pSE420Q vector (WO 2006-132145) to prepare pSQCABB2, an expression plasmid carrying the CaBDHB gene.

### Example 30

### Expression of CaBDHB Gene in E. Coli

The plasmid pSQCABB2 prepared in Example 29 was introduced into E. coli JM109 by the Hanahan method to obtain a transformant E. coli JM109 (pSQCABB2). This transformant was cultured by the method described in Example 2.

The culture obtained by the above-mentioned method was dispensed into a 2 ml Eppendorf tube, and centrifuged to collect the cells. Under anaerobic conditions, 50 mM MOPS buffer (pH 7.0) containing 1 mM DTT was added to the cells, and subjected to ultrasonication to disrupt the cells. Intact cells and cell debris were removed by centrifugation to obtain a cell-free extract.

The CaBDHB activity of the obtained cell-free extract was measured by the BDH activity assay using butylaldehyde as a substrate, and determined to be 0.0971 U/mg.

CaBDHB is known to act on acetaldehyde and butylaldehyde. However, its activity on 3-hydroxybutylaldehyde has never been reported and is therefore unknown. Thus, the CaBDHB activity was measured using prepared 3-hydroxybutylaldehyde (3-hydroxybutylaldehyde:acetaldehyde=3:1) as a substrate. As a result, the activity was 0.0462 U/mg, which was found to be 48% relative to the activity on butylaldehyde taken as 100%,

### Example 31

### Plasmid Construction for Fermentative Production of 1,3-BG-2

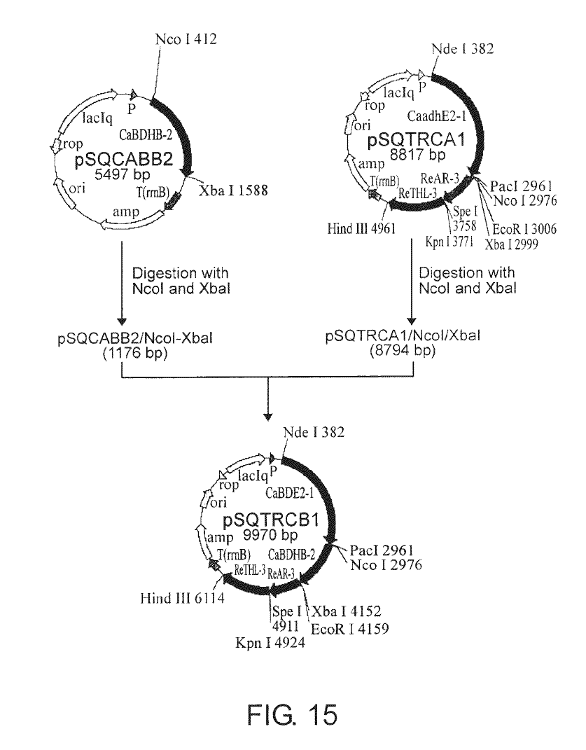

In order to construct plasmid 2 for fermentative production of 1,3-BG, the CaBDHB gene in the pSQCABB plasmid produced in Example 29 was subcloned into pSQTRCA1 by the method shown in FIG. 15. Specifically, pSQCABB2 was double-digested with NcoI and XbaI, and ligated with NcoI-XbaI-treated pSQTRCA1 vector to construct pSQTRCB1, a coexpression plasmid carrying the ReTHL gene, ReAR1 gene, CaAdhE gene, and CaBDHB gene.

### Example 32

### Expression of ReTHL Gene, ReAR1 Gene, CaAdhE Gene, and CaBDHB Gene in E. Coli

The plasmid pSQTRCB1 prepared in Example 31 was introduced into E. coli W3110 by electroporation to obtain a transformant E. coli W3110 (pSQTRCB1). This transformant was cultured by the method described in Example 2.

The culture obtained by the above-mentioned method was dispensed into a 2 ml Eppendorf tube, and centrifuged to collect the cells. Under anaerobic conditions, 50 mM MOPS buffer (pH 7.0) containing 1 mM DTT was added to the cells, and subjected to ultrasonication to disrupt the cells. Intact cells and cell debris were removed by centrifugation to obtain a cell-free extract.

The obtained cell-free extract was assayed for ReTHL activity by TEL activity assay-1, for ReAR1 activity by the 3HBD activity assay, for BCDH activity of CaAdhE by the BCDH activity assay (substrate: butyryl-CoA), and for CaBDHB activity by the BDH activity assay (substrate: butylaldehyde), and the results were 10.0 U/mg, 2.08 U/mg, 0.0298 U/mg, and 0.124 U/mg, respectively,

### Example 33

### Expression of ReTHL Gene, ReAR1 Gene, and CaAdhE Gene in E. coli-2

The plasmid pSQTHRA1 prepared in Example 27 was introduced into E. coli HB101 by electroporation to obtain a transformant E. coli HB101 (pSQTHRA1), This transformant was cultured by the method described in Example 2.

The culture obtained by the above-mentioned method was dispensed into a 2 ml Eppendorf tube, and centrifuged to collect the cells. Under anaerobic conditions, 50 mM MOPS buffer (pH 7.0) containing 1 mM DTT was added to the cells, and subjected to ultrasonication to disrupt the cells. Intact cells and cell debris were removed by centrifugation to obtain a cell-free extract.

The obtained cell-free extract was assayed for ReTHL activity by THL activity assay-1, for ReAR1 activity by the 3HBD activity assay, and for BCDH activity of CaAdhE by the BCDH activity assay (substrate: butyryl-CoA) and the BDH activity assay (butylaldehyde), and the results were 7.36 U/mg, 1.18 U/mg, 0.0434 U/mg and 0.151 U/mg, respectively.

### Example 34

### Production of 1,3-BG by Fermentation

The Objective of this Example is to produce 1,3-BG from glucose by fermentation using E. coli JM109 (pSQTRCA1),

Transformant E. coli JM109 (pSQTRCA1) was inoculated into a 21 mm diameter test tube containing 7 mL of LB medium consisting of 10 g/L tryptone, 5 g/L, yeast extract, 10 g/L NaCl, and 50 mg/L ampicillin, which had been adjusted to pH 7.2. The cells were pre-cultured under aerobic conditions at 30° C. while shaking at 250 rpm for 18 hours.

The pre-culture was inoculated into a 500-mL baffled flask containing 50 L of liquid medium consisting of 20 g/L tryptone, 10 g/L yeast extract 10 g/L NaCl, and 50 mg/L ampicillin, which had been adjusted to pH 7.0. The cells were cultured at 30.0° C. while shaking at 140 rpm for 18 hours. The induction of expression was performed using 0.02 mM IPTG.

Fermentation liquid 1: Washed cells obtained from the above main culture of E. coli JM109 (pSQTRCA1) were added to a 100-mL baffled flask with 10 mL of a solution containing 56 g/L glucose and liquid medium consisting of 20 g/L tryptone, 10 g/L yeast extract, and 10 g/L NaCl, which had been adjusted to pH 7.0, such that the inoculation ratio was 100%. The flask was sealed with a silicone plug, and fermentation was performed under non-aerated conditions at 30° C. while shaking at 100 rpm for 72 hours.

Fermentation liquid 2: 10-fold concentrated washed cells from the above main culture of E. coli JM109 (pSQTRCA1) were added to a 100-mL baffled flask with 10 mL of a solution containing 56 g/L glucose and MES buffer (pH 6.0) such that the inoculation, ratio was 100%. The flask was sealed with a silicone plug, and fermentation was performed under non-aerated conditions at 30° C. while shaking at 100 rpm for 72 hours.

After 72 hours of the beginning of fermentation, 2 ml of the fermentation liquid was sampled and centrifuged to remove the cells and insoluble materials. The supernatant was filtered through Millex-LH (Millipore), and the filtrate was analyzed by HPLC under the same conditions as in Example 22. The concentration of produced 1,3-BG is shown in Table 1. E. coli JM109 carrying no plasmid was also used for fermentation under the same conditions, but found to produce no detectable 1,3-BG.

TABLE 1
1,3-BG production by E. coli JM109 (pSQTRCA1)

| | Fermentation | 1,3-BG | Molar yield |
|---|---|---|---|
| No. | liquid | (g/L)¹⁾ | (%)²⁾ |
| Fermentation liquid 1 | 2 x YT | 0.560 | 2.0 |
| Fermentation liquid 2 | MES (pH 6.0) | 0.819 | 2.9 |

¹⁾calculated by HPLC
²⁾molar yield (%) = 100 × ((produced 1,3-BG [mM])/(initial glucose concentration [mM]))

### Example 35

### Production of 1,3-BG by Fermentation—2

The pre-culture and main culture of E. coli HB101 (pSQTRCA1) and E. coli HB101 (pSQTRCB1) were carried out by the methods described in Example 34.

Fermentation liquid 3: 10-fold concentrated washed cells from the main culture of E. coli HB101 (pSQTRCA1) were added to a 500-mL baffled flask with 100 mL of a solution containing 30 g/L glucose, 0.02 mM IPTG, 100 mM HEPES buffer, and M9 medium consisting of 6.8 g/L, Na₂HPO₄, 3.0 g/L KH₂PO₄, 0.5 g/L NaCl, 1.0 g/L NH₄Cl, 493 mg/L MgSO₄.7H₂O, and 14.7 mg/L CaCl₂.2H₂O, which had been adjusted to pH 7.5, such that the inoculation ratio was 20%. The flask was sealed with a silicone plug, and fermentation was performed at 30° C. while shaking at 140 rpm for 48 hours.

Fermentation liquid 4: Fermentation was performed in the same manner as fermentation liquid 3 except that the fermentation temperature was 37° C.

Fermentation liquid 5: Fermentation was performed in the same manner as fermentation liquid 4 except that the inoculated transformant was E. coli HB101 (pSQTRCB1).

After 48 hours of the beginning of fermentation, 2 ml of the fermentation liquid was sampled and centrifuged to remove the cells and insoluble materials. The supernatant was filtered through Millex-LH (Millipore), and the filtrate was analyzed by HPLC under the same conditions as in Example 22. The concentration of produced 1,3-BG is shown in Table 2.

TABLE 2
1,3-BG production by E. coli HB101 (pSQTRCA1)
and E. coli HB101 (pSQTRCB1)

| | | 1,3-BG | Molar yield |
|---|---|---|---|
| No. | Strain | (g/L)¹⁾ | (%)²⁾ |
| Fermentation liquid 3 | W3110(pSQTRCA1) | 0.435 | 2.9 |
| Fermentation liquid 4 | W3110(pSQTRCA1) | 1.028 | 6.9 |
| Fermentation liquid 5 | W3110(pSQTRCB1) | 0.075 | 0.5 |

¹⁾calculated by HPLC
²⁾molar yield (%) = 100 × ((produced 1,3-BG [mM])/(initial glucose concentration [mM]))

### Example 36

### Determination of Optical Purity of Produced 1,3-BG

After 48 hours of fermentation, an organic layer was extracted from a 1-mL sample of fermentation liquid 4 with ethyl acetate, and salted out using sodium chloride. After concentration of the obtained extract, 0.1 mL acetyl chloride was added and reacted at 25° C. for 10 minutes. The reacted solution was neutralized using saturated sodium bicarbonate, and then an organic layer was extracted with 1 mL hexane. HPLC analysis of this extract showed that the optical purity of (R)-1,3-BG was 86.6%.

The HPLC conditions were as follows:

HPLC column: CHIRALCEL (4.6 mm×250 mm) manufactured by Daicel Chemical Industries, Ltd.

Eluent: hexane:isopropanol=19:1

Column temperature: 40° C.

Flow rate: 1.0 mL/mm

Detection: LTV absorption at 220 nm

Under the above conditions, (S)-1,3-BG and (R)-1,3-BG was eluted at 6.8 min and 8.5 min, respectively. The optical purity was determined based on the 220-nm UV absorption of each fraction.

### INDUSTRIAL APPLICABILITY

The present invention provides recombinant microorganisms capable of producing 1,3-butanediol efficiently from carbohydrate materials such as glucose, which are derived from renewable resources. In addition, methods for producing 1,3-butanediol using such microorganisms are provided. The methods of the present invention are industrially advantageous in that cheaper materials can be used to produce 1,3-butanediol. The present invention enables the production of 1,3-butanediol from renewable resources.

1. A recombinant microorganism in which the enzymatic activity of (1) shown below is enhanced, wherein the microorganism produces 1,3-alkyldiol represented by Formula 2 from a fermentation substrate:
(1) activity of an enzyme that catalyzes production of 3-hydroxyalkylaldehyde by reducing 3-hydroxyacyl-CoA using NADH and/or NADPH as a coenzyme, as shown by Formula 1: 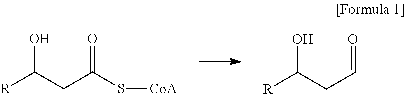
(wherein R represents a alkyl group or hydrogen; and CoA represents coenzyme A) 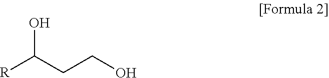
(wherein R represents a C₁₋₃ alkyl group or hydrogen).

2. The recombinant microorganism of claim 1, wherein R is methyl in Formulas 1 and 2 recited in claim 1, and the microorganism produces 1,3-butanediol from a fermentation substrate.

3. The recombinant microorganism of claim 2, wherein 1,3-butanediol produced from the fermentation substrate recited in claim 2 is (R)-1,3-butanediol.

4. The recombinant microorganism of claim 1, wherein the enzyme catalyzing the reaction of Formula 1 recited in claim 1 (1) is classified as EC1.2.1.10 under the international classification of enzyme.

5. The recombinant microorganism of claim 1, wherein the enzyme catalyzing the reaction of Formula 1 recited in claim 1 (1) is any one of:
(a) a protein comprising the amino acid sequence of SEQ ID NO: 1, 65, or 67;
(b) an enzyme comprising an amino acid sequence in which one or more amino acids are substituted, deleted, inserted, or added in the amino acid sequence of SEQ ID NO: 1, 65, or 67;
(c) an enzyme comprising an amino acid sequence encoded by the nucleotide sequence of SEQ ID NO: 2, 66, or 68;
(d) an enzyme comprising an amino acid sequence encoded by a DNA which hybridizes under stringent conditions with a DNA comprising the nucleotide sequence of SEQ ID NO: 2, 66, or 68; and
(e) a protein having an identity of 85% or higher with the amino acid sequence of SEQ ID NO: 1, 65, or 67.

6. The recombinant microorganism of claim 1, in which the enzymatic activity of (2) shown below is enhanced in addition to the enzymatic activity of (1) recited in claim 1, wherein the microorganism produces 1,3-alkyldiol represented by Formula 2 from a fermentation substrate:
(1) activity of an enzyme that catalyzes production of 3-hydroxyalkylaldehyde by reducing 3-hydroxyacyl-CoA using NADH and/or NADPH as a coenzyme, as shown by Formula 1; and
(2) activity of an enzyme that catalyzes production of 1,3-alkyldiol represented by Formula 2 by reducing 3-hydroxyalkylaldehyde using NADH and/or NADPH as a coenzyme, as shown by Formula 3 (Formula 3 does not show two reactions, but only shows the production of alcohol from aldehyde): 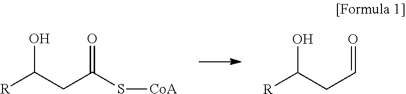
(wherein R represents a C₁₋₃ alkyl group or hydrogen; and CoA represents coenzyme A) 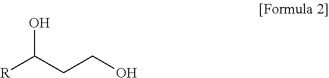
(wherein R represents a C₁₋₃ group or hydrogen) 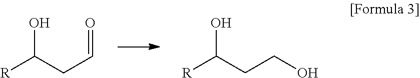
(wherein R represents a C₁₋₃ alkyl group or hydrogen).

7. The recombinant microorganism of claim 6, wherein R is methyl in Formulas 1 to 3 recited in claim 6, and the microorganism produces 1,3-butanediol from a fermentation substrate.

8. The recombinant microorganism of claim 7, wherein 1,3-butanediol produced from the fermentation substrate recited in claim 7 is (R)-1,3-butanediol.

9. The recombinant microorganism of claim 6, wherein the enzyme catalyzing the reaction or Formula 3 recited in claim 6 is any one of:
(a) a protein comprising the amino acid sequence of SEQ ID NO: 3, 5, or 7;
(b) an enzyme comprising an amino acid sequence in which one or more amino acids are substituted, deleted, inserted, or added in the amino acid sequence of SEQ ID NO: 3, 5, or 7;
(c) an enzyme comprising an amino acid sequence encoded by the nucleotide sequence of SEQ ID NO: 4, 6, or 8;
(d) an enzyme comprising an amino acid sequence encoded by a DNA whim hybridizes under stringent conditions with a DNA comprising the nucleotide sequence of SEQ ID NO: 4, 6, or 8; and
(e) an enzyme having an identity of 85% or higher with the amino acid sequence of SEQ ID NO: 3, 5, or 7.

10. The recombinant microorganism of claim 1, in which the enzymatic activity of (3) shown below is enhanced in addition to the enzymatic activity of (1) recited in claim 1, wherein the microorganism produces 1,3-alkyldiol represented by Formula 2 from a fermentation substrate:
(1) activity of an enzyme that catalyzes production of 3-hydroxyalkylaldehyde by reducing 3-hydroxyacyl-CoA using NADH and/or NADPH as a coenzyme, as shown by Formula 1: and
(3) activity of an enzyme that produces 3-hydroxyacyl-CoA by reducing 3-oxoacyl-CoA in an NADH- and/or NADPH-dependent manner as shown by Formula 4: 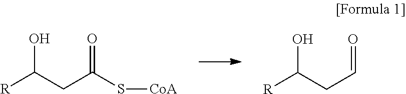
(wherein R represents a C₁₋₃ alkyl group or hydrogen; and CoA represents coenzyme A) 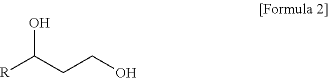
(wherein R represents a C₁₋₃ alkyl group or hydrogen) 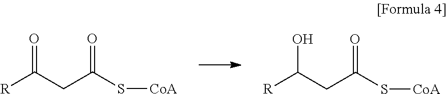
(wherein R represents a C₁₋₃ alkyl group or hydrogen; and CoA represents coenzyme A).

11. The recombinant microorganism of claim 10, wherein R is methyl in Formulas 1 and 2 recited in claim 10, and the microorganism produces (R)-1,3-butanediol represented by Formula 5 from a fermentation substrate:

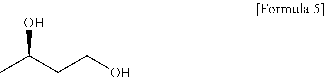

12. The recombinant microorganism of claim 10, wherein the enzyme catalyzing the reaction of Formula 4 recited in claim 10 is an R-form-specific reductase, and is any one of:
(a) a protein comprising the amino acid sequence of SEQ ID NO: 9, 11, 13, 15, or 17;
(b) an enzyme comprising an amino acid sequence in which one or more amino acids are substituted, deleted, inserted, or added in the amino acid sequence of SEQ ID NO: 9, 11, 13, 15, or 17;
(c) an enzyme comprising an amino acid sequence encoded by the nucleotide sequence of SEQ ID NO: 10, 12, 14, 16, or 18;
(d) an enzyme comprising an amino acid sequence encoded by a DNA which hybridizes under stringent conditions with a DNA comprising the nucleotide sequence of SEQ ID NO: 10, 12, 14, 16, or 18, and
(e) an enzyme having an identity of 85% or higher with the amino acid sequence of SEQ ID NO: 9, 11, 13, 15, or 17.

13. A method for producing a diol compound represented by Formula 2, comprising the steps of:
contacting a fermentation substrate with at least one active material selected from the group consisting of a culture of the recombinant microorganism of claim 1, a cell of the recombinant microorganism, and a processed product thereof; and
collecting 1,3-alkyodiol represented by Formula 2: 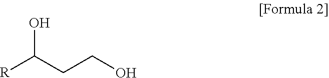
(wherein R represents a C₁₋₃ alkyl group or hydrogen).

14. The method of claim 13, wherein the diol compound is (R)-1,3-butanediol represented by Formula 5

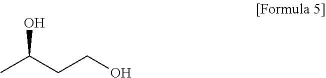

15. The method of claim 13, wherein the culturing of the recombinant microorganism and the production of the diol compound are carried out separately.


SEQUENCE LISTING (72 sequences)

SEQ ID NO: 1 - protein, 858 aa, Clostridium acetobutylicum
MKVTNQKELKQKLNELREAQKKFATYTQEQVDKIFKQCAIAAAKERINLAKLAVEETGIGLVEDKIIKNHFAAEYIYNKYKNEKTCGIIDHDDSLGITKVAEPIGIVAAIVPTTNPTSTAIFKSLISLKTRNAIFFSPHPRAKKSTIAAAKLILDAAVKAGAPKNIIGWIDEPSIELSQDLMSEADIILATGGPSMVKAAYSSGKPAIGVGAGNTPAIIDESADIDMAVSSIILSKTYDNGVICASEQSILVMNSIYEKVKEEFVKRGSYILNQNEIAKIKETMFKNGAINADIVGKSAYIIAKMAGIEVPQTTKILIGEVQSVEKSELFSHEKLSPVLAMYKVKDFDEALKKAQRLIELGGSGHTSSLYIDSQNNKDKVKEFGLAMKTSRTFINMPSSQGASGDLYNFAIAPSFTLGCGTWGGNSVSQNVEPKHLLNIKSVAERRENMLWFKVPQKIYFKYGCLRFALKELKDMNKKRAFIVTDKDLFKLGYVNKITKVLDEIDIKYSIFTDIKSDPTIDSVKKGAKEMLNFEPDTIISIGGGSPMDAAKVMHLLYEYPEAEIENLAINFMDIRKRICNFPKLGTKAISVAIPTTAGTGSEATPFAVITNDETGMKYPLTSYELTPNMAIIDTELMLNMPRKLTAATGIDALVHAIEAYVSVMATDYTDELALRAIKMIFKYLPRAYKNGTNDIEAREKMAHASNIAGMAFANAFLGVCHSMAHKLGAMHHVPHGIACAVLIEEVIKYNATDCPTKQTAFPQYKSPNAKRKYAEIAEYLNLKGTSDTEKVTALIEAISKLKIDLSIPQNISAAGINKKDFYNTLDKMSELAFDDQCTTANPRYPLISELKDIYIKSF

SEQ ID NO: 2 - DNA, 2577 bp, Clostridium acetobutylicum
atgaaagttacaaatcaaaaagaactaaaacaaaagctaaatgaattgagagaagcgcaaaagaagtttgcaacctatactcaagagcaagttgataaaatttttaaacaatgtgccatagccgcagctaaagaaagaataaacttagctaaattagcagtagaagaaacaggaataggtcttgtagaagataaaattataaaaaatcattttgcagcagaatatatatacaataaatataaaaatgaaaaaacttgtggcataatagaccatgacgattctttaggcataacaaaggttgctgaaccaattggaattgttgcagccatagttcctactactaatccaacttccacagcaattttcaaatcattaatttctttaaaaacaagaaacgcaatattcttttcaccacatccacgtgcaaaaaaatctacaattgctgcagcaaaattaattttagatgcagctgttaaagcaggagcacctaaaaatataataggctggatagatgagccatcaatagaactttctcaagatttgatgagtgaagctgatataatattagcaacaggaggtccttcaatggttaaagcggcctattcatctggaaaacctgcaattggtgttggagcaggaaatacaccagcaataatagatgagagtgcagatatagatatggcagtaagctccataattttatcaaagacttatgacaatggagtaatatgcgcttctgaacaatcaatattagttatgaattcaatatacgaaaaagttaaagaggaatttgtaaaacgaggatcatatatactcaatcaaaatgaaatagctaaaataaaagaaactatgtttaaaaatggagctattaatgctgacatagttggaaaatctgcttatataattgctaaaatggcaggaattgaagttcctcaaactacaaagatacttataggcgaagtacaatctgttgaaaaaagcgagctgttctcacatgaaaaactatcaccagtacttgcaatgtataaagttaaggattttgatgaagctctaaaaaaggcacaaaggctaatagaattaggtggaagtggacacacgtcatctttatatatagattcacaaaacaataaggataaagttaaagaatttggattagcaatgaaaacttcaaggacatttattaacatgccttcttcacagggagcaagcggagatttatacaattttgcgatagcaccatcatttactcttggatgcggcacttggggaggaaactctgtatcgcaaaatgtagagcctaaacatttattaaatattaaaagtgttgctgaaagaagggaaaatatgctttggtttaaagtgccacaaaaaatatattttaaatatggatgtcttagatttgcattaaaagaattaaaagatatgaataagaaaagagcctttatagtaacagataaagatctttttaaacttggatatgttaataaaataacaaaggtactagatgagatagatattaaatacagtatatttacagatattaaatctgatccaactattgattcagtaaaaaaaggtgctaaagaaatgcttaactttgaacctgatactataatctctattggtggtggatcgccaatggatgcagcaaaggttatgcacttgttatatgaatatccagaagcagaaattgaaaatctagctataaactttatggatataagaaagagaatatgcaatttccctaaattaggtacaaaggcgatttcagtagctattcctacaactgctggtaccggttcagaggcaacaccttttgcagttataactaatgatgaaacaggaatgaaataccctttaacttcttatgaattgaccccaaacatggcaataatagatactgaattaatgttaaatatgcctagaaaattaacagcagcaactggaatagatgcattagttcatgctatagaagcatatgtttcggttatggctacggattatactgatgaattagccttaagagcaataaaaatgatatttaaatatttgcctagagcctataaaaatgggactaacgacattgaagcaagagaaaaaatggcacatgcctctaatattgcggggatggcatttgcaaatgctttcttaggtgtatgccattcaatggctcataaacttggggcaatgcatcacgttccacatggaattgcttgtgctgtattaatagaagaagttattaaatataacgctacagactgtccaacaaagcaaacagcattccctcaatataaatctcctaatgctaagagaaaatatgctgaaattgcagagtatttgaatttaaagggtactagcgataccgaaaaggtaacagccttaatagaagctatttcaaagttaaagatagatttgagtattccacaaaatataagtgccgctggaataaataaaaaagatttttataatacgctagataaaatgtcagagcttgcttttgatgaccaatgtacaacagctaatcctaggtatccacttataagtgaacttaaggatatctatataaaatcattttaa

SEQ ID NO: 3 - protein, 390 aa, Clostridium acetobutylicum
VVDFEYSIPTRIFFGKDKINVLGRELKKYGSKVLIVYGGGSIKRNGIYDKAVSILEKNSIKFYELAGVEPNPRVTTVEKGVKICRENGVEVVLAIGGGSAIDCAKVIAAACEYDGNPWDIVLDGSKIKRVLPIASILTIAATGSEMDTWAVINNMDTNEKLIAAHPDMAPKFSILDPTYTYTVPTNQTAAGTADIMSHIFEVYFSNTKTAYLQDRMAEALLRTCIKYGGIALEKPDDYEARANLMWASSLAINGLLTYGKDTNWSVHLMEHELSAYYDITHGVGLAILTPNWMEYILNNDTVYKFVEYGVNVWGIDKEKNHYDIAHQAIQKTRDYFVNVLGLPSRLRDVGIEEEKLDIMAKESVKLTGGTIGNLRPVNASEVLQIFKKSV

SEQ ID NO: 4 - DNA, 1173 bp, Clostridium acetobutylicum
gtggttgatttcgaatattcaataccaactagaatttttttcggtaaagataagataaatgtacttggaagagagcttaaaaaatatggttctaaagtgcttatagtttatggtggaggaagtataaagagaaatggaatatatgataaagctgtaagtatacttgaaaaaaacagtattaaattttatgaacttgcaggagtagagccaaatccaagagtaactacagttgaaaaaggagttaaaatatgtagagaaaatggagttgaagtagtactagctataggtggaggaagtgcaatagattgcgcaaaggttatagcagcagcatgtgaatatgatggaaatccatgggatattgtgttagatggctcaaaaataaaaagggtgcttcctatagctagtatattaaccattgctgcaacaggatcagaaatggatacgtgggcagtaataaataatatggatacaaacgaaaaactaattgcggcacatccagatatggctcctaagttttctatattagatccaacgtatacgtataccgtacctaccaatcaaacagcagcaggaacagctgatattatgagtcatatatttgaggtgtattttagtaatacaaaaacagcatatttgcaggatagaatggcagaagcgttattaagaacttgtattaaatatggaggaatagctcttgagaagccggatgattatgaggcaagagccaatctaatgtgggcttcaagtcttgcgataaatggacttttaacatatggtaaagacactaattggagtgtacacttaatggaacatgaattaagtgcttattacgacataacacacggcgtagggcttgcaattttaacacctaattggatggagtatattttaaataatgatacagtgtacaagtttgttgaatatggtgtaaatgtttggggaatagacaaagaaaaaaatcactatgacatagcacatcaagcaatacaaaaaacaagagattactttgtaaatgtactaggtttaccatctagactgagagatgttggaattgaagaagaaaaattggacataatggcaaaggaatcagtaaagcttacaggaggaaccataggaaacctaagaccagtaaacgcctccgaagtcctacaaatattcaaaaaatctgtgtaa

SEQ ID NO: 5 - protein, 337 aa, Bacillus stearothermophilus
MKAAVVEQFKKPLQVKEVEKPKISYGEVLVRIKACGVCHTDLHAAHGDWPVKPKLPLIPGHEGVGVIEEVGPGVTHLKVGDRVGIPWLYSACGHCDYCLSGQETLCERQQNAGYSVDGGYAEYCRAAADYVVKIPDNLSFEEAAPIFCAGVTTYKALKVTGAKPGEWVAIYGIGGLGHVAVQYAKAMGLNVVAVDLGDEKLELAKQLGADLVVNPKHDDAAQWIKEKVGGVHATVVTAVSKAAFESAYKSIRRGGACVLVGLPPEEIPIPIFDTVLNGVKIIGSIVGTRKDLQEALQFAAEGKVKTIVEVQPLENINDVFDRMLKGQINGRVVLKVD

SEQ ID NO: 6 - DNA, 1014 bp, Bacillus stearothermophilus
atgaaagctgcagttgtggaacaatttaaaaagccgttacaagtgaaagaagtggaaaaacctaagatctcatacggggaagtattagtgcgcatcaaagcgtgtggggtatgccatacagacttgcatgccgcacatggcgactggcctgtaaagcctaaactgcctctcattcctggccatgaaggcgtcggtgtaattgaagaagtaggtcctggggtaacacatttaaaagttggagatcgcgtaggtatcccttggctttattcggcgtgcggtcattgtgactattgcttaagcggacaagaaacattatgcgaacgtcaacaaaacgctggctattccgtcgatggtggttatgctgaatattgccgtgctgcagccgattatgtcgtaaaaattcctgataacttatcgtttgaagaagccgctccaatcttttgcgctggtgtaacaacatataaagcgctcaaagtaacaggcgcaaaaccaggtgaatgggtagccatttacggtatcggcgggcttggacatgtcgcagtccaatacgcaaaggcgatggggttaaacgtcgttgctgtcgatttaggtgatgaaaaacttgagcttgctaaacaacttggtgcagatcttgtcgtcaatccgaaacatgatgatgcagcacaatggataaaagaaaaagtgggcggtgtgcatgcgactgtcgtcacagctgtttcaaaagccgcgttcgaatcagcctacaaatccattcgtcgcggtggtgcttgcgtactcgtcggattaccgccggaagaaatacctattccaattttcgatacagtattaaatggagtaaaaattattggttctatcgttggtacgcgcaaagacttacaagaggcacttcaatttgcagcagaaggaaaagtaaaaacaattgtcgaagtgcaaccgcttgaaaacattaacgacgtattcgatcgtatgttaaaagggcaaattaacggccgcgtcgtgttaaaagtagattaa

SEQ ID NO: 7 - protein, 348 aa, Saccharomyces cerevisiae
MSIPETQKAIIFYESNGKLEHKDIPVPKPKPNELLINVKYSGVCHTDLHAWHGDWPLPTKLPLVGGHEGAGVVVGMGENVKGWKIGDYAGIKWLNGSCMACEYCELGNESNCPHADLSGYTHDGSFQEYATADAVQAAHIPQGTDLAEVAPILCAGITVYKALKSANLRAGHWAAISGAAGGLGSLAVQYAKAMGYRVLGIDGGPGKEELFTSLGGEVFIDFTKEKDIVSAVVKATNGGAHGIINVSVSEAAIEASTRYCRANGTVVLVGLPAGAKCSSDVFNHVVKSISIVGSYVGNRADTREALDFFARGLVKSPIKVVGLSSLPEIYEKMEKGQIAGRYVVDTSK

SEQ ID NO: 8 - DNA, 1047 bp, Saccharomyces cerevisiae
atgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtcgttacgttgttgacacttctaaataa

SEQ ID NO: 9 - protein, 246 aa, Ralstonia eutropha
MTQRIAYVTGGMGGIGTAICQRLAKDGFRVVAGCGPNSPRREKWLEQQKALGFDFIASEGNVADWDSTKTAFDKVKSEVGEVDVLINNAGITRDVVFRKMTRADWDAVIDTNLTSLFNVTKQVIDGMADRGWGRIVNISSVNGQKGQFGQTNYSTAKAGLHGFTMALAQEVATKGVTVNTVSPGYIATDMVKAIRQDVLDKIVATIPVKRLGLPEEIASICAWLSSEESGFSTGADFSLNGGLHMG

SEQ ID NO: 10 - DNA, 741 bp, Ralstonia eutropha
atgactcagcgcattgcgtatgtgaccggcggcatgggtggtatcggaaccgccatttgccagcggctggccaaggatggctttcgtgtggtggccggttgcggccccaactcgccgcgccgcgaaaagtggctggagcagcagaaggccctgggcttcgatttcattgcctcggaaggcaatgtggctgactgggactcgaccaagaccgcattcgacaaggtcaagtccgaggtcggcgaggttgatgtgctgatcaacaacgccggtatcacccgcgacgtggtgttccgcaagatgacccgcgccgactgggatgcggtgatcgacaccaacctgacctcgctgttcaacgtcaccaagcaggtgatcgacggcatggccgaccgtggctggggccgcatcgtcaacatctcgtcggtgaacgggcagaagggccagttcggccagaccaactactccaccgccaaggccggcctgcatggcttcaccatggcactggcgcaggaagtggcgaccaagggcgtgaccgtcaacacggtctctccgggctatatcgccaccgacatggtcaaggcgatccgccaggacgtgctcgacaagatcgtcgcgacgatcccggtcaagcgcctgggcctgccggaagagatcgcctcgatctgcgcctggttgtcgtcggaggagtccggtttctcgaccggcgccgacttctcgctcaacggcggcctgcatatgggctga

SEQ ID NO: 11 - protein, 241 aa, Zoogloea ramigera
MSRVALVTGGSRGIGAAISIALKAAGYKVAASYAGNDDAANAFKAETGIAVYKWDVSSYEACVEGIAKVEADLGPIDVLVNNAGITKDAMFHKMTPDQWNAVINTNLTGLFNMTHPVWSGMRDRSFGRIVNISSINGQKGQMGQANYSAAKAGDLGFTKALAQEGAAKGITVNAICPGYIGTEMVRAIPEKVLNERIIPQIPVGRLGEPDEIARIVVFLASDEAGFITGSTISANGGQFFV

SEQ ID NO: 12 - DNA, 726 bp, Zoogloea ramigera
atgagtcgtgtagcattggtaacggggggatcgcggggcatcggcgcagccatttcgattgcgctgaaggcggcgggatacaaggtggctgccagctatgccggcaatgacgatgcggccaacgccttcaaggcggaaacgggcatcgccgtctacaagtgggacgtgtcgagctacgaggcctgcgtcgaggggatcgccaaggtggaggccgatctcggcccgattgacgttctcgtcaacaatgcgggcatcaccaaggacgcgatgttccacaagatgacgcccgaccagtggaatgcggtcatcaacaccaacctcacgggtctcttcaacatgacccatccggtctggtccggcatgcgcgaccgcagcttcggccgcatcgtcaacatctcctcgatcaacggccagaagggccagatgggtcaggcgaactattccgccgccaaggccggcgacctcggcttcaccaaggcgctggcgcaggaaggcgcggccaagggcatcacggtcaacgccatctgccccggctatatcggtacggaaatggtgcgcgccattccggaaaaggtgctgaacgagcggatcatcccgcagatcccggtcggccgcctcggcgagccggacgagatcgcccgcatcgtcgtcttcctcgcctcggacgaggccggcttcatcaccggctcgaccatctcggcgaacggcggccagttcttcgtctaa

SEQ ID NO: 13 - protein, 254 aa, Bacillus stearothermophilus
MSQRFAGRVAFVTGGSRGIGKAIVTRFAEEGAKVAFIDLNEEALEATAAELREKGYDVYAKVASVTDREQVETTMQEVVDRFGSLDILVNNAGVIRDNLLFKMTDDDWQTVMDVHLKGAFYCARAAQKYMVEKGYGRIINISSTSALGNRGQANYSAAKAGIQGFTKTLAIELGKFGITTNAIAPGFIETDMTKATAERLGISFEQLIQASVANIPVGRSGRPEDIAHAVAFFADERSSFVNGQVLYVAGGPKC

SEQ ID NO: 14 - DNA, 765 bp, Bacillus stearothermophilus
atgtctcaacgttttgcaggtcgtgtggcgtttgtgaccggaggaagccgcggcatcggcaaggcgattgtcacccgcttcgcggaagaaggggcgaaagtggcattcatcgatttaaatgaagaagcgcttgaagcgacggctgccgagctgcgggagaaagggtatgacgtgtacgccaaagtggcgagcgtcaccgaccgcgaacaagtggaaacgacgatgcaagaggtcgtcgaccggttcggctcgcttgatattcttgtcaacaacgccggcgtcatccgcgacaacttgctctttaaaatgacggacgacgactggcaaacggtcatggacgttcatttgaaaggggcgttttattgcgcccgcgccgcgcaaaaatatatggtcgaaaaagggtacgggcgcatcatcaacatttcgtcgacctccgcgctcggaaaccgcggccaggcgaactattcggcggcgaaagccggcattcaagggtttacgaaaacgttggcgatcgagctcggcaagttcggcattacgacgaacgccatcgctccaggattcattgaaaccgatatgacaaaggcaaccgctgagcggcttggcatttcgtttgagcagctcattcaggcgagcgtcgccaacatcccggtcggacgcagtggccgtccggaagacatcgcccatgcagtcgcgtttttcgccgacgaacggtcgtcgtttgtcaacggccaagtgctgtatgtggcaggtggtccaaaatgttaa

SEQ ID NO: 15 - protein, 261 aa, Streptomyces violaceoruber
MATQDSEVALVTGATSGIGLEIARRLGKEGLRVFVCARGEEGLRTTLKELREAGVEADGRTCDVRSVPEIEALVAAVVERYGPVDVLVNNAGRPGGGATAELADELWLDVVETNLTGVFRVTKQVLKAGGMLERGTGRIVNIASTGGKQGVVHAAPYSASKHGVVGFTKALGLELARTGITVNAVCPGFVETPMAASVREHYSDIWEVSTEEAFDRITARVPIGRYVQPSEVAEMVAYLIGPGAAAVTAQALNVCGGLGNY

SEQ ID NO: 16 - DNA, 786 bp, Streptomyces violaceoruber
atggccacgcaggactccgaagtcgcactggtgacgggtgcgaccagcggaatcgggctggagatcgcccgcaggctcggtaaggaggggttgcgcgtgttcgtctgcgcgcgcggcgaagaaggactgcggacgacgctgaaggagttgcgggaggcgggcgtggaggcggacgggcggacctgcgacgtccgctcggtcccggagatcgaggctctcgtggcggcggtggtcgagcgttacggtccggtcgacgtgctggtgaacaacgcgggccggcccggcggcggcgccacggccgaactcgccgacgaactctggctcgatgtcgtggagaccaacctcaccggcgtgttccgggtgaccaaacaggtcctcaaggcgggcggcatgctcgaacggggcacgggccgaatcgtcaacatcgcctcgaccggcggaaagcagggtgtggtgcacgccgcgccctactccgcctcgaagcacggcgtggtcggcttcaccaaggcgctcggtctcgaactggcgaggaccggcatcacggtgaacgccgtctgccccggattcgtcgagacgccgatggccgcgtccgtgcgcgagcactactcggacatctgggaggtgtcgaccgaggaggccttcgaccggatcaccgcgcgcgtgccgatcggccggtacgtgcagccgtccgaggtggcggagatggtggcgtacctgatcgggcccggtgcggccgcggtcaccgcgcaggcgctgaacgtctgcggcgggctggggaactactaa

SEQ ID NO: 17 - protein, 254 aa, Pichia finlandica
MSYNFHNKVAVVTGALSGIGLSVAKKFLQLGAKVTISDVSGEKKYHETVVALKAQNLNTDNLHYVQADSSKEEDNKKLISETLATFGGLDIVCANAGIGKFAPTHETPFDVWKKVIAVNLNGVFLLDKLAINYWLEKSKPGVIVNMGSVHSFVAAPGLAHYGAAKGGVKLLTQTLALEYASHGIRVNSVNPGYISTPLIDEVPKERLDKLVSLHPIGRLGRPEEVADAVAFLCSQEATFINGVSLPVDGGYTAQ

SEQ ID NO: 18 - DNA, 765 bp, Pichia finlandica
atgtcttataatttccataacaaggttgcagttgttactggagctctatcaggaatcggcttaagcgtcgcaaaaaagttccttcagctcggcgccaaagtaacgatctctgatgtcagtggagagaaaaaatatcacgagactgttgttgctctgaaagcccaaaatctcaacactgacaacctccattatgtacaggcagattccagcaaagaagaagataacaagaaattgatttcggaaactctggcaacctttgggggcctggatattgtttgtgctaatgcaggaattggaaagttcgctcccacccatgaaacacccttcgacgtatggaagaaggtgattgctgtgaatttgaatggagtattcttactggataagctagccatcaattactggctagagaaaagcaaacccggcgtaattgtcaacatgggatcagtccactcttttgtagcagctcctggccttgcgcattatggagctgcaaaaggcggtgtcaaactgttaacacaaacattggctctggagtacgcatctcatggtattcgtgtaaattctgtcaatccggggtacatttcgactcctttgatagatgaggttccgaaagagcggttggataaacttgtaagcctgcaccctattgggcgtctgggtcgtccagaggaagttgctgatgcagtcgcatttctgtgttcccaggaggccactttcatcaacggcgtttctttgccggttgacggtgggtacacagcacaataa

SEQ ID NO: 19 - protein, 282 aa, Clostridium acetobutylicum
MKKVCVIGAGTMGSGIAQAFAAKGFEVVLRDIKDEFVDRGLDFINKNLSKLVKKGKIEEATKVEILTRISGTVDLNMAADCDLVIEAAVERMDIKKQIFADLDNICKPETILASNTSSLSITEVASATKRPDKVIGMHFFNPAPVMKLVEVIRGIATSQETFDAVKETSIAIGKDPVEVAEAPGFVVNRILIPMINEAVGILAEGIASVEDIDKAMKLGANHPMGPLELGDFIGLDICLAIMDVLYSETGDSKYRPHTLLKKYVRAGWLGRKSGKGFYDYSK

SEQ ID NO: 20 - DNA, 849 bp, Clostridium acetobutylicum
atgaaaaaggtatgtgttataggtgcaggtactatgggttcaggaattgctcaggcatttgcagctaaaggatttgaagtagtattaagagatattaaagatgaatttgttgatagaggattagattttatcaataaaaatctttctaaattagttaaaaaaggaaagatagaagaagctactaaagttgaaatcttaactagaatttccggaacagttgaccttaatatggcagctgattgcgatttagttatagaagcagctgttgaaagaatggatattaaaaagcagatttttgctgacttagacaatatatgcaagccagaaacaattcttgcatcaaatacatcatcactttcaataacagaagtggcatcagcaactaaaagacctgataaggttataggtatgcatttctttaatccagctcctgttatgaagcttgtagaggtaataagaggaatagctacatcacaagaaacttttgatgcagttaaagagacatctatagcaataggaaaagatcctgtagaagtagcagaagcaccaggatttgttgtaaatagaatattaataccaatgattaatgaagcagttggtatattagcagaaggaatagcttcagtagaagacatagataaagctatgaaacttggagctaatcacccaatgggaccattagaattaggtgattttataggtcttgatatatgtcttgctataatggatgttttatactcagaaactggagattctaagtatagaccacatacattacttaagaagtatgtaagagcaggatggcttggaagaaaatcaggaaaaggtttctacgattattcaaaataa

SEQ ID NO: 21 - protein, 393 aa, Ralstonia eutropha
MTDVVIVSAARTAVGKFGGSLAKIPAPELGAVVIKAALERAGVKPEQVSEVIMGQVLTAGSGQNPARQAAIKAGLPAMVPAMTINKVCGSGLKAVMLAANAIMAGDAEIVVAGGQENMSAAPHVLPGSRDGFRMGDAKLVDTMIVDGLWDVYNQYHMGITAENVAKEYGITREAQDEFAVGSQNKAEAAQKAGKFDEEIVPVLIPQRKGDPVAFKTDEFVRQGATLDSMSGLKPAFDKAGTVTAANASGLNDGAAAVVVMSAAKAKELGLTPLATIKSYANAGVDPKVMGMGPVPASKRALSRAEWTPQDLDLMEINEAFAAQALAVHQQMGWDTSKVNVNGGAIAIGHPIGASGCRILVTLLHEMKRRDAKKGLASLCIGGGMGVALAVERK

SEQ ID NO: 22 - DNA, 1182 bp, Ralstonia eutropha
atgactgacgttgtcatcgtatccgccgcccgcaccgcggtcggcaagtttggcggctcgctggccaagatcccggcaccggaactgggtgccgtggtcatcaaggccgcgctggagcgcgccggcgtcaagccggagcaggtgagcgaagtcatcatgggccaggtgctgaccgccggttcgggccagaaccccgcacgccaggccgcgatcaaggccggcctgccggcgatggtgccggccatgaccatcaacaaggtgtgcggctcgggcctgaaggccgtgatgctggccgccaacgcgatcatggcgggcgacgccgagatcgtggtggccggcggccaggaaaacatgagcgccgccccgcacgtgctgccgggctcgcgcgatggtttccgcatgggcgatgccaagctggtcgacaccatgatcgtcgacggcctgtgggacgtgtacaaccagtaccacatgggcatcaccgccgagaacgtggccaaggaatacggcatcacacgcgaggcgcaggatgagttcgccgtcggctcgcagaacaaggccgaagccgcgcagaaggccggcaagtttgacgaagagatcgtcccggtgctgatcccgcagcgcaagggcgacccggtggccttcaagaccgacgagttcgtgcgccagggcgccacgctggacagcatgtccggcctcaagcccgccttcgacaaggccggcacggtgaccgcggccaacgcctcgggcctgaacgacggcgccgccgcggtggtggtgatgtcggcggccaaggccaaggaactgggcctgaccccgctggccacgatcaagagctatgccaacgccggtgtcgatcccaaggtgatgggcatgggcccggtgccggcctccaagcgcgccctgtcgcgcgccgagtggaccccgcaagacctggacctgatggagatcaacgaggcctttgccgcgcaggcgctggcggtgcaccagcagatgggctgggacacctccaaggtcaatgtgaacggcggcgccatcgccatcggccacccgatcggcgcgtcgggctgccgtatcctggtgacgctgctgcacgagatgaagcgccgtgacgcgaagaagggcctggcctcgctgtgcatcggcggcggcatgggcgtggcgctggcagtcgagcgcaaataa

SEQ ID NO: 23 - protein, 393 aa, Zoogloea ramigera
MSTPSIVIASAARTAVGSFNGAFANTPAHELGATVISAVLERAGVAAGEVNEVILGQVLPAGEGQNPARQAAMKAGVPQEATAWGMNQLCGSGLRAVALGMQQIATGDASIIVAGGMESMSMAPHCAHLRGGVKMGDFKMIDTMIKDGLTDAFYGYHMGTTAENVAKQWQLSRDEQDAFAVASQNKAEAAQKDGRFKDEIVPFIVKGRKGDITVDADEYIRHGATLDSMAKLRPAFDKEGTVTAGNASGLNDGAAAALLMSEAEASRRGIQPLGRIVSWATVGVDPKVMGTGPIPASRKALERAGWKIGDLDLVEANEAFAAQACAVNKDLGWDPSIVNVNGGAIAIGHPIGASGARILNTLLFEMKRRGARKGLATLCIGGGMGVAMCIESL

SEQ ID NO: 24 - DNA, 1182 bp, Zoogloea ramigera
atgagtactccatcaatcgtcatcgccagcgccgcccgcaccgcggtcggttccttcaacggcgctttcgccaacacgcccgcccatgaactcggggcgaccgtgatttcggcggttctcgagcgcgcgggcgttgcggcgggcgaggtgaacgaggtgattctcggccaggtgctgccggccggcgaaggccagaacccggcccgccaggccgccatgaaggccggcgtgccgcaggaggcgaccgcctggggcatgaaccagctttgcggctcgggcctgcgcgccgtcgcgctcggcatgcagcagatcgccacgggcgatgcgagcatcatcgtcgccggcggcatggaatccatgtcaatggccccgcattgcgcgcatctgcgcggcggcgtgaagatgggcgatttcaagatgatcgacacgatgatcaaggacggcctgaccgacgccttctacggctaccacatgggcacgaccgccgagaatgtcgccaagcagtggcagctttcccgcgacgagcaggacgccttcgccgtcgcctcgcagaacaaggccgaggccgcccagaaggacggccgcttcaaggacgagatcgttcccttcatcgtcaagggccgcaagggcgacatcacggtcgatgccgacgaatatatccgccacggcgcgacgctcgattcaatggcgaagctccgcccggccttcgacaaggaaggcacggtgacggccggcaacgcctccggcctcaatgacggcgcggccgcggccctcctgatgagcgaagcggaagcctcgcgccgcggcatccagccgctcggccgcatcgtttcctgggcgacggtcggcgtcgatcccaaggtcatgggcaccggcccgatcccggcctcccgcaaggcgctcgagcgcgccggctggaagatcggcgatctcgacctcgtggaagccaacgaagccttcgcggcgcaggcctgcgcggtcaacaaggacctcggctgggatccgtccatcgtcaacgtcaacggcggtgccatcgccatcggccacccgatcggcgcgtccggcgcccgcatcctcaacacgctcctcttcgagatgaagcgtcgcggcgcccgcaagggtctcgccacgctctgcatcggcggcggcatgggcgtggcgatgtgcatcgaaagtctttaa

SEQ ID NO: 25 - protein, 394 aa, Escherichia coli
MKNCVIVSAVRTAIGSFNGSLASTSAIDLGATVIKAAIERAKIDSQHVDEVIMGNVLQAGLGQNPARQALLKSGLAETVCGFTVNKVCGSGLKSVALAAQAIQAGQAQSIVAGGMENMSLAPYLLDAKARSGYRLGDGQVYDVILRDGLMCATHGYHMGITAENVAKEYGITREMQDELALHSQRKAAAAIESGAFTAEIVPVNVVTRKKTFVFSQDEFPKANSTAEALGALRPAFDKAGTVTAGNASGINDGAAALVIMEESAALAAGLTPLARIKSYASGGVPPALMGMGPVPATQKALQLAGLQLADIDLIEANEAFAAQFLAVGKNLGFDSEKVNVNGGAIALGHPIGASGARILVTLLHAMQARDKTLGLATLCIGGGQGIAMVIERLN

SEQ ID NO: 26 - DNA, 1185 bp, Escherichia coli
atgaaaaattgtgtcatcgtcagtgcggtacgtactgctatcggtagttttaacggttcactcgcttccaccagcgccatcgacctgggggcgacagtaattaaagccgccattgaacgtgcaaaaatcgattcacaacacgttgatgaagtgattatgggtaacgtgttacaagccgggctggggcaaaatccggcgcgtcaggcactgttaaaaagcgggctggcagaaacggtgtgcggattcacggtcaataaagtatgtggttcgggtcttaaaagtgtggcgcttgccgcccaggccattcaggcaggtcaggcgcagagcattgtggcggggggtatggaaaatatgagtttagccccctacttactcgatgcaaaagcacgctctggttatcgtcttggagacggacaggtttatgacgtaatcctgcgcgatggcctgatgtgcgccacccatggttatcatatggggattaccgccgaaaacgtggctaaagagtacggaattacccgtgaaatgcaggatgaactggcgctacattcacagcgtaaagcggcagccgcaattgagtccggtgcttttacagccgaaatcgtcccggtaaatgttgtcactcgaaagaaaaccttcgtcttcagtcaagacgaattcccgaaagcgaattcaacggctgaagcgttaggtgcattgcgcccggccttcgataaagcaggaacagtcaccgctgggaacgcgtctggtattaacgacggtgctgccgctctggtgattatggaagaatctgcggcgctggcagcaggccttacccccctggctcgcattaaaagttatgccagcggtggcgtgccccccgcattgatgggtatggggccagtacctgccacgcaaaaagcgttacaactggcggggctgcaactggcggatattgatctcattgaggctaatgaagcatttgctgcacagttccttgccgttgggaaaaacctgggctttgattctgagaaagtgaatgtcaacggcggggccatcgcgctcgggcatcctatcggtgccagtggtgctcgtattctggtcacactattacatgccatgcaggcacgcgataaaacgctggggctggcaacactgtgcattggcggcggtcagggaattgcgatggtgattgaacgcttgaattaa

SEQ ID NO: 27 - DNA, 38 bp, Artificial
  An artificially synthesized primer sequence
gacggtacctatatatgactgatgttgtcatcgtatcc

SEQ ID NO: 28 - DNA, 34 bp, Artificial
  An artificially synthesized primer sequence
cacaagcttattatttacgttcaactgccagcgc

SEQ ID NO: 29 - DNA, 35 bp, Artificial
  An artificially synthesized primer sequence
gacggtacctatatatgagtactccatcaatcgtc

SEQ ID NO: 30 - DNA, 34 bp, Artificial
  An artificially synthesized primer sequence
cacaagcttattaaagactttcgatgcacatcgc

SEQ ID NO: 31 - DNA, 22 bp, Artificial
  An artificially synthesized primer sequence
ggaatccatgtcaatggccccg

SEQ ID NO: 32 - DNA, 20 bp, Artificial
  An artificially synthesized primer sequence
gctcgattcaatggcgaagc

SEQ ID NO: 33 - DNA, 22 bp, Artificial
  An artificially synthesized primer sequence
caatgcggggccattgacatgg

SEQ ID NO: 34 - DNA, 23 bp, Artificial
  An artificially synthesized primer sequence
cggagcttcgccattgaatcgag

SEQ ID NO: 35 - DNA, 37 bp, Artificial
  An artificially synthesized primer sequence
gacggtacctatatatgaaaaattgtgtcatcgtcag

SEQ ID NO: 36 - DNA, 35 bp, Artificial
  An artificially synthesized primer sequence
cacaagcttattaattcaagcgttcaatcaccatc

SEQ ID NO: 37 - DNA, 37 bp, Artificial
  An artificially synthesized primer sequence
gaggaattcatatatgactcaacgtattgcgtatgtg

SEQ ID NO: 38 - DNA, 29 bp, Artificial
  An artificially synthesized primer sequence
cagactagtattagcccatgtgcaggccg

SEQ ID NO: 39 - DNA, 23 bp, Artificial
  An artificially synthesized primer sequence
catggcttcactatggcactggc

SEQ ID NO: 40 - DNA, 23 bp, Artificial
  An artificially synthesized primer sequence
gccagtgccatagtgaagccatg

SEQ ID NO: 41 - DNA, 36 bp, Artificial
  An artificially synthesized primer sequence
gaggaattcatatatgagtcgtgtagcattggtaac

SEQ ID NO: 42 - DNA, 29 bp, Artificial
  An artificially synthesized primer sequence
cagactagtattagacgaagaactggccg

SEQ ID NO: 43 - DNA, 31 bp, Artificial
  An artificially synthesized primer sequence
gaggaattcatatatggccacgcaggactcc

SEQ ID NO: 44 - DNA, 29 bp, Artificial
  An artificially synthesized primer sequence
cagactagtattagtagttccccagcccg

SEQ ID NO: 45 - DNA, 35 bp, Artificial
  An artificially synthesized primer sequence
gaggaattcatatatgtctcaacgttttgcaggtc

SEQ ID NO: 46 - DNA, 31 bp, Artificial
  An artificially synthesized primer sequence
cagactagtattaacattttggaccacctgc

SEQ ID NO: 47 - DNA, 39 bp, Artificial
  An artificially synthesized primer sequence
gaggaattcatatatgaaaaaggtatgtgttataggtgc

SEQ ID NO: 48 - DNA, 40 bp, Artificial
  An artificially synthesized primer sequence
cagactagtattattttgaataatcgtagaaaccttttcc

SEQ ID NO: 49 - DNA, 42 bp, Artificial
  An artificially synthesized primer sequence
gaggaattctaaaatgtcttataatttccataacaaggttgc

SEQ ID NO: 50 - DNA, 38 bp, Artificial
  An artificially synthesized primer sequence
tcgactagtattattgtgctgtgtacccaccgtcaacc

SEQ ID NO: 51 - DNA, 39 bp, Artificial
  An artificially synthesized primer sequence
ggctctggagtacgcatctcatggtattcgtgtaaattc

SEQ ID NO: 52 - DNA, 39 bp, Artificial
  An artificially synthesized primer sequence
gaatttacacgaataccatgagatgcgtactccagagcc

SEQ ID NO: 53 - DNA, 34 bp, Artificial
  An artificially synthesized primer sequence
gtaagcctgcaccctattgggcgtctgggtcgtc

SEQ ID NO: 54 - DNA, 34 bp, Artificial
  An artificially synthesized primer sequence
gacgacccagacgcccaatagggtgcaggcttac

SEQ ID NO: 55 - DNA, 37 bp, Artificial
  An artificially synthesized primer sequence
gaccatatgaaagttacaaatcaaaaagaactaaaac

SEQ ID NO: 56 - DNA, 40 bp, Artificial
  An artificially synthesized primer sequence
ctgttaattaaaatgattttatatagatatccttaagttc

SEQ ID NO: 57 - DNA, 21 bp, Artificial
  An artificially synthesized primer sequence
ctatagaagcatacgtttcgg

SEQ ID NO: 58 - DNA, 21 bp, Artificial
  An artificially synthesized primer sequence
ccgaaacgtatgcttctatag

SEQ ID NO: 59 - DNA, 39 bp, Artificial
  An artificially synthesized primer sequence
ggaccatggttgatttcgaatattcaataccaactagaa

SEQ ID NO: 60 - DNA, 47 bp, Artificial
  An artificially synthesized primer sequence
cgatctagaattacacagattttttgaatatttgtaggacttcggag

SEQ ID NO: 61 - DNA, 24 bp, Artificial
  An artificially synthesized primer sequence
gatggaaatccgtgggatattgtg

SEQ ID NO: 62 - DNA, 24 bp, Artificial
  An artificially synthesized primer sequence
cacaatatcccacggatttccatc

SEQ ID NO: 63 - DNA, 27 bp, Artificial
  An artificially synthesized primer sequence
gtttaccatctcgtctgcgtgatgttg

SEQ ID NO: 64 - DNA, 27 bp, Artificial
  An artificially synthesized primer sequence
caacatcacgcagacgagatggtaaac

SEQ ID NO: 65 - protein, 874 aa, Thermoanaerobacter pseudethanolicus
MPNLLQERREVKEKTEVKETLDVKRQIDQLVEKAQRAQEKFMSYTQEQIDKIVKAMALAGIENHVRLAKLAHEETKMGVYEDKITKNLFAVEYVYNYIKDKKTVGVLSENLEENYMEVAEPVGVIAGVTPVTNPTSTTMFKSLIAIKTRNPIIFSFHPKALKCSIETAKTMYEAALKAGAPEGCIGWIETPSIEATQLLMTHPGVSLILATGGAGMVKAAYSSGKPALGVGPGNVPCYIEKTANIKRAVSDLILSKTFDNGTVCASEQAVIIDEEIADEVKKLMKEYGCYFLNKEETKMLEEFAIDKNTGLMNPAVVGQPATKIAEMAGFKVPENTKILVAEYPAVGPEYPLSREKLSPILALYTVKDYNEGIKRCEEMTEFGGLGHSAVIHSENQQIINEFAKRVRAGRILVNAPASQGAIGDIYNTAIPSLTLGCGTMGRNSTTDNVSVYNLINIKRVFIRKERMKWFRVPPQIYFERGSLQYLSQVKGKKAFIVTDPVMVKLGFVDKVTYQLDKANIKYEIFSEVEPDPSVDTVEKGVKIMRGFEPDLLIAVGGGSAIDAAKGMWLFYEYPDTKFEDLRLKFMDIRKRTYRFPELGKKALFIAIPTTSGSGTGSEVTAFAVITDKKKNIKYPLTDYELTPDIAIIDPDLTMTIPPSVTADTGMDALTHAIEAYVSVMASDYTDALAEKAIKLIFEYLPKAYKNGQDKVAREKMHNASCIAGMAFTNAFLGINHSMAHILGAKFHLPHGRANAILLPYVIEYNAELPKKFASFPQYEYPKAAEKYAEIAKFLGLPASTIEEGVKSLIEAIKNLMKELNLPLTLKEAGINKEEFEKQIMEMSDIAFNDQCTGSNPRMPLVSEIAEIYRKAYGE

SEQ ID NO: 66 - DNA, 2625 bp, Thermoanaerobacter pseudethanolicus
atgcctaacttattacaagaaagaagagaagtaaaagaaaaaacagaagtgaaagaaacgctggatgttaaaagacaaatagaccaattagtagaaaaagcacaaagagcacaagaaaaattcatgtcttatacccaagaacaaatagataaaatagttaaagctatggctttggctggtattgaaaatcatgtgagattggcaaaattagcccatgaagaaacaaaaatgggtgtttatgaagacaaaataacgaaaaatctttttgcagtagaatatgtgtacaattatataaaagacaaaaagactgtcggcgtccttagtgaaaacttagaagaaaattatatggaagtggcagaacctgtaggagttatagccggcgtaacacccgttacaaaccctacttctacaacaatgttcaaaagcttaatcgctattaaaacaagaaatccaataatttttagctttcacccaaaagcattaaaatgtagtatagaaacagcaaagaccatgtacgaggcagcattaaaagctggtgcaccagaaggatgcataggttggattgaaacgccttcaattgaagcaacgcaacttctcatgacacatcccggcgtctcccttatcctcgcaacaggcggcgctggaatggtaaaagccgcttacagctcaggaaaacccgcccttggcgtaggtcctggcaatgtcccctgctacattgaaaaaactgcaaatattaaaagagcagtctcagacctaatacttagcaaaacttttgacaatggcacagtatgcgcatcagaacaagctgtgataatagacgaagaaatagcagatgaagtgaaaaaattaatgaaagaatatggttgctattttctcaataaagaagagactaaaatgctagaagaattcgctatagacaaaaacaccggtttaatgaatccagcagttgtaggtcaaccggcaacaaaaattgctgaaatggctggatttaaagtacctgaaaacacaaaaatacttgtagctgaatatcctgctgtaggaccagaatatcctctctccagagaaaagctaagtccaatacttgctttatacacagtcaaagattacaacgaaggcataaaaagatgtgaagaaatgacagaattcggcggacttggtcactctgcagtaatacattctgaaaaccaacaaattattaatgaatttgcaaaaagagtacgagcaggaagaattcttgtaaacgcccctgcttctcaaggagctattggagatatttataacacggctatcccatctttgactttgggttgcggtacaatgggtagaaattcaacaacagataatgtaagtgtttataaccttataaacataaaacgagtttttataagaaaagagagaatgaaatggttcagagttccaccacaaatttattttgaaagagggtcattacaatatctgtcacaagtaaaaggcaaaaaagcctttattgtaacagaccctgtgatggttaaattaggttttgtagataaagtaacgtatcaattagataaggcaaatattaaatatgaaatattctctgaagttgaaccagatccttcagttgacactgtagaaaaaggggtaaaaataatgagagggtttgagcctgacttactaatagctgtaggtggtggttctgcaatagatgcagcgaagggaatgtggcttttctacgaataccctgatacaaaattcgaagacttaaggttaaaatttatggacataagaaaaagaacttatagatttcctgaactggggaaaaaagctttatttatcgcaataccaaccaccagcggtagcggtacaggttctgaagttactgcctttgcagttataacagacaaaaagaaaaatataaagtaccctctcacagattacgaattaacaccagatatcgctataatagaccctgacctcacaatgacaataccaccctctgtcacagcagacactggtatggacgctttaacccatgctattgaagcctatgtatctgtaatggcatcagactatacagacgctctcgcagaaaaagctataaaattgatatttgaatatctacctaaagcatacaaaaacgggcaagataaagtcgcccgcgaaaagatgcacaatgcttcatgcatagctggaatggccttcacaaatgcatttttaggaataaaccatagtatggctcatatacttggtgctaaattccatcttccacacggaagagcaaatgcaatacttctgccctatgtcatagaatacaatgctgagttacctaaaaaatttgcttcattcccacaatatgaatatcccaaagcagccgagaaatatgcagaaattgcaaaattcttgggacttcctgcatcaaccattgaagaaggagtaaaaagcctcattgaagcaataaaaaatcttatgaaagagcttaatcttcctttgacacttaaagaagcaggcataaataaagaagaattcgaaaaacaaataatggaaatgtctgatatagcctttaatgaccagtgcaccggttcaaacccaagaatgccattggtatccgaaattgcagaaatatatagaaaagcctatggagaataa

SEQ ID NO: 67 - protein, 378 aa, Propionibacterium freudenreichii subsp. freudenreichii
MDFSLTEDQQLMVDGFTELMNSEAWEKYFHECDENSEYPERWVKAICDLGFDRILLPEEYDGLGLGWQTLAAAYEALGRAGGPTYVLYQLPGWDTVIREGTEEQKKDILKFVGSGKQMLNYAMTEPSAGSSWDDMSTTYSRKGGKVYLNGHKTFITSSMKVPYLVVMARDADNMGTYTEWFVDMSLPGITKEPLHKLGLRMDSCADIYFDNVELREEDLFGTEGNGFKRGVKDFDLERFEVAMTNYGTAYCAFEDAAKYANQRVQGGEAIARKQLIQLKFADMKVDITNMRNMLYEIAWKADNDQLGRGDCSMAKYYCSHAAARVVDNALQTLAGVGVTGEHRVQRFYRDLRVDRVSGGTDEMMILAAGRGALRDYRS

SEQ ID NO: 68 - DNA, 1137 bp, Propionibacterium freudenreichii subsp. freudenreichii
atggatttctcattgaccgaagaccagcagctcatggtggacggcttcaccgagctgatgaacagtgaggcatgggagaagtacttccacgagtgtgacgagaactcggagtatcccgagcgctgggtcaaggcgatctgcgacctcggcttcgaccgcatcctgctgcccgaggaatacgacggcctgggcctgggctggcagacgttggccgccgcctatgaggcgctcggccgcgctggtggccccacctatgtgctctaccaactgcccggctgggacacggtgatccgcgaaggcaccgaggaacagaagaaggacatcctcaagttcgtgggcagcgggaagcagatgctcaactacgccatgaccgagcccagcgccgggtcgagctgggacgacatgtccaccacctacagccgcaagggcggcaaggtgtacctcaacggacacaagaccttcatcacctcatccatgaaggtgccctacctcgtggtgatggcccgcgacgccgacaacatgggcacctacaccgagtggttcgtcgacatgagccttccgggcatcaccaaggagccgctgcacaagctcggcctgcgcatggattcgtgcgccgacatctacttcgacaacgttgaactgcgcgaggaagacctgttcggcaccgagggcaacggcttcaagcgcggcgtgaaggacttcgacctggagcgcttcgaggtggccatgaccaactacggcaccgcctactgcgccttcgaggatgcagcgaagtacgccaaccagcgcgtgcagggaggcgaggcgattgcccgcaagcagctgatccagctgaagttcgccgacatgaaggtggacatcaccaatatgcgcaacatgctctacgagattgcctggaaggccgacaacgaccagctgggccgcggcgactgctccatggcgaagtactactgctcccacgccgcggcccgcgtggtcgacaacgcgctgcagaccctcgccggcgtgggcgtcaccggtgaacaccgcgtgcagcgcttctaccgcgacctccgcgtggaccgcgtctccggtggaaccgacgaaatgatgatcctcgccgccggccgtggtgcactgcgcgactaccgcagctga

SEQ ID NO: 69 - DNA, 44 bp, Artificial
  An artificially synthesized primer sequence
gaccatatgcctaacttattacaagaacgccgcgaagtaaaaga

SEQ ID NO: 70 - DNA, 45 bp, Artificial
  An artificially synthesized primer sequence
gctgttaattaattattctccataggctttgcgatatatttctgc

SEQ ID NO: 71 - DNA, 36 bp, Artificial
  An artificially synthesized primer sequence
gaccatatggatttctcattgaccgaagaccagcag

SEQ ID NO: 72 - DNA, 35 bp, Artificial
  An artificially synthesized primer sequence
gctgttaattaactacggtagtcgcgcagtgcacc